SEQUENCE LISTING (14 sequences)

SEQ ID NO: 1 - DNA, 8500 bp, Homo sapiens
  source 1..8500: mol_type = genomic DNA
ggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgagataccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtggatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtaaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtgtggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcagctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacataacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacagcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctgaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattccagggaccactgggtgagagaggaataaggcccagagcgaggaaaggattttaccaaagcatcaatacaaccagcccaaccatcggtccacacctgggcatttggtgagagtcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggaatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgagtttgttattttgctttgaattgttgagtctgaatttcaccaaagccaatcatttgaacaaagcggggaatgttgggataggaaaggtaagtagggatagtggtcaagtgggaggggtggaaaggagactaaagactgggagagagggaagcactttttttaaataaagttgaacacacttgggaaaagcttacaggccaggcctgtaatcccaacactttgggaggccaaggtgggaggatagcttaaccccaggagtttgagaccagcctgagcaacatagtgagaacttgtctctacagaaaaaaaaaaaaaaaaaaatttaattaggcaagcgtggtagtgcgcacctgtcgtcccagctactcaggaggctgaggtaggaaaatcactggagcccaggagttagaggttacagtgagctatgatcacactactgcactccagcctgggcaacagagggagaccctgtctctaaataaaaaaagaaaagaaaaaaaaagcttacaacttgagattcagcatcttgctcagtatttccaagactaatagattatggtttaaaagatgcttttatactcattttctaatgcaactcctagaaactctatgatatagttgaggtaagtattgttaccacacatgggctaagatccccagaggcagactgcctgagttcaattcttggctccaccattcccaagttccctaacctctctatgcctcagtttcctcttctgtaaagtagggacactcatacttctcatttcagaacatttttgtgaagaataaattatgttatccatttgaggcccttagaatggtacccggtgtatattaagtgctagtacatgttagctatcatcattatcactttatatgagatggactggggttcatagaaacccaatgacttgattgtggctactactcaataaataatagaatttggatttaaa

SEQ ID NO: 2 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 3 - DNA, 2358 bp, Homo sapiens
  source 1..2358: mol_type = genomic DNA
attcggccgaaggagctacgcgggccacgctgctggctggcctgacctaggcgcgcggggtcgggcggccgcgcgggcgggctgagtgagcaagacaagacactcaagaagagcgagctgcgcctgggtcccggccaggcttgcacgcagaggcgggcggcagacggtgcccggcggaatctcctgagctccgccgcccagctctggtgccagcgcccagtggccgccgcttcgaaagtgactggtgcctcgccgcctcctctcggtgcgggaccatgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccactgagagagacttgtgctcaaggaatcggctggggactgctacctctgagaagacacaaggtgatttcagactgcagaggggaaagacttccatctagtcacaaagactccttcgtccccagttgccgtctaggattgggcctcccataattgctttgccaaaataccagagccttcaagtgccaaacagagtatgtccgatggtatctgggtaagaagaaagcaaaagcaagggaccttcatgcccttctgattcccctccaccaaaccccacttcccctcataagtttgtttaaacacttatcttctggattagaatgccggttaaattccatatgctccaggatctttgactgaaaaaaaaaaagaagaagaagaaggagagcaagaaggaaagatttgtgaactggaagaaagcaacaaagattgagaagccatgtactcaagtaccaccaagggatctgccattgggaccctccagtgctggatttgatgagttaactgtgaaataccacaagcctgagaactgaattttgggacttctacccagatggaaaaataacaactatttttgttgttgttgtttgtaaatgcctcttaaattatatatttattttattctatgtatgttaatttatttagtttttaacaatctaacaataatatttcaagtgcctagactgttactttggcaatttcctggccctccactcctcatccccacaatctggcttagtgccacccacctttgccacaaagctaggatggttctgtgacccatctgtagtaatttattgtctgtctacatttctgcagatcttccgtggtcagagtgccactgcgggagctctgtatggtcaggatgtaggggttaacttggtcagagccactctatgagttggacttcagtcttgcctaggcgattttgtctaccatttgtgttttgaaagcccaaggtgctgatgtcaaagtgtaacagatatcagtgtctccccgtgtcctctccctgccaagtctcagaagaggttgggcttccatgcctgtagctttcctggtccctcacccccatggccccaggcccacagcgtgggaactcactttcccttgtgtcaagacatttctctaactcctgccattcttctggtgctactccatgcaggggtcagtgcagcagaggacagtctggagaaggtattagcaaagcaaaaggctgagaaggaacagggaacattggagctgactgttcttggtaactgattacctgccaattgctaccgagaaggttggaggtggggaaggctttgtataatcccacccacctcaccaaaacgatgaagttatgctgtcatggtcctttctggaagtttctggtgccatttctgaactgttacaacttgtatttccaaacctggttcatatttatactttgcaatccaaataaagataacccttattccata

SEQ ID NO: 4 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 5 - DNA, 6850 bp, Homo sapiens
  source 1..6850: mol_type = genomic DNA
aactgtgctatggagtagaagcaggaggttttcaacctagtcacagagcagcacctaccccctcctcctttccacacctgcaaactcttttacttgggctgaatatttagtgtaattacatctcagctttgagggctcctgtggcaaattcccggattaaaaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattactattgtatacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaatattaaaactatcatgtgacttcatgtaatcaggctgaacatttctacaattactagatgtattagacgtaagtattttctttagttaaaccacccatgttagaaatgttttctgtagaatttataaacaactatcaatgcagacaatttaataagcctggggatgatttacttacagtaaacatttatcaaattgtacatttgtgctatcaacaattaataagcaaatatgtgaaaatagtttctgtcttctatgaagttagatatttgatggttaaaacccctataaatcatagtttcatatgggaaaaaataattgaaatacagtgtaaatttaaataatttattaagtatagcaaataattgaaatatggtggactaaattttgtcatagaaatatgtgcaagttatagtagtggctcacatgagaggtaatcaattctgctaatagtagcagaatgagtgcagtggaacatgaaaaacttgaggagataacagttgaggtgggtttccatagatgcataatagttcaagagcaagatttggtggggaggcactattcaagacagggactaagttcaaaatccaagacgtatgctgggacacacctctgacaggttggcataaaggaggcttaatcaaactatttttcttcttctgaaacagaagcaataattttcatttacatttgacatatcccgaggtaatattaacattagggaaagttactcttttccatctttccacattcttgcaggaccataaaatctgaattttccagtatttttaataagagggaagaaacctctctttttcttctctttttcatctcccaagagatcctcctctcatgactacagttgaataggtggtttctattggaagacattcaggaattcaaggtgcatgtccataaatggactttttttgttgttgttcagagctggaccttgaatgatgcatccttctctctgttgtaaccatgaataatgcacccttcatgctatagcctttaacgattcacccttcttattgtaaccttgaatgattcaccctttatggtgtagccttgagtgacgcacccttcatgttgtagccttcaatgatgcacactccatgttatagccttgaatgatataccctttatgctgcagcctttctcttatggggaaaagcctgcagatatcctgctgcttaactgacaagtgtggtgagaaataagtagaaatctaaagaggggaagaccattttggacacttatctgcaaggcagatccaacacacttttccagtagtcaagctacttctaattttgttcagtatcaaaatgagaaacaggcctgattctccagcactcttgtcaacacaacttccccccatatttatatatatacacacacatatatatctttatatatatacacacatatatatctttatatatatatatttatatatatatctttttgcatatatacatatatatgtatctttatttcctttgaaataaagataaatatagctgatttctttggcttcgacacttactatttgcatgactaagggaagctagttaacctttctgtgactcatttccttgtccataaaatgggaatattaattgtacatgtcttatggattggtgtgtgaattcagttagcgagtgtagaatataacttatagatcaaagtagagtaaatggaaagggctcaactatggtgttgctactgccattgttattacaggcacacagttcgagctataatcatttcaagggaaattcttatgtgtcagttctggatcgaggtctgagattctgcatttcaaacaaacttccaggaatgctgctgcttcttggtccacacttggagaaataagtcagcagagagtcctctcgtttcctattgtaccatgtctgtcttttgtctcctgcttattggcctctgtaaggaactcacagctgctataataaagtaccaaaaactgggtggcttaaaacaacagaaacttactttctcacaattctggaggctaaaaattcaaaatcaaggtgtcagcagggtcagattccttccaaagcctttaggagaggacctttccttgctcctcctaggtttctggttaaatctaggtgttctttgccttatggcagcgtgactctaatttatgcctccatcttcaccctcacatggccttctctctcatgtgtttgtgtcttttctcttcttcttctatgaacatgagttatattggattaaggctcaccctaatttagtatgaacccatcttaacttgattatatctgcaaagactctatttccaaatgaagtcagactcacaggtattgggggtttgatattgaacatatcttctgggaggagacacaacttaatcattaatatccactttcttttttccttattaaatgtttaatttttttgttttcattaaaactgttgttcgattatgggtgcttcacataaaaggttggaaacttaaaaatttgtctctgacccctcctggttggaaaggcctctgttgtacatttatgctagcctaggccataccactttctgtcttcagtacagccatcttagtttattcaagtgacacagattttccagaacacagtattcatgatcttttaaagcatttttcttcaaagactttgatctggcaataaatgttactatgtaattctcatgacataaattaggcataacttggatctcctcttcttctgctcattcatttgtctgaatcatcactattatcttttttatattcctttaccgttctccataatgcttttccaagaaactggtttattccacaattttattgcagaggcagctgcaggatatcataatcttatctttatacgaaggaagaattgcctaatccctcaagtaaactaaaaatgttttatacagtcatttctcattcatccaatgttccaggcagtcccagtccaagactgccccttcacacacacaacaactcttcacaagacttcactgtccttcagactctcctgcagcacagacattcgaggttgctgagtcgacattgcagttatttcactcttgattttgctgctcaattttaagtttccacacttattgctaaaacattccttctagtggatctgtttatgtattcaagcacaccatctgtcacaattatttcaaatcacaggtctgtgacagactgtcaatttcacaccacaaagtataatagtggaaaacaaagtaaaatcaagaaagggagaacactaaacttattaaaaccatatacaagcatacgcattcaggaaactaaaacaaccttcaaagaagttgaacaattttgagccaaaagaaagggcatatgcacaccctaccccagtataagataaccagaaaaggactgggtgagttgcctcctgagtattatgactgccatcaatcacttaggcattggagatcaggagactcttcccaggccacacattctgcagtgactgtgagggctgaatgaatgcttccaggccatcaggaacaagcatagaaacacaaatcaattttaaaatacactcaaaaatgtcaaaataatccaacttttttgtcttgatcagatatatctactagcacatattacttcattttttcagccaatatttattaataacctactgtgtgcttgacagtgttctagtggcagagacacagcagtgcataatgccttgccctctgaagcagatgttgcagcaggaggagacattccaggacatgacaattaaagtggaggaatgtcaagcagaactggggaataagggatgtgaagggggtgagacttgctattttatataggatggtcggaaaaaggacttatcaggagtgacatttgagcagggagatgaagaactgaagggagcaagccaagaaaagaggccctgaatttggaaaatacatggcacgttccaacaagagcagagaggacaaacgtgagcagagtaaagtgagcaagaagagataatatatgatacaaggtcagaaaggtaatgacaatcgcatcctgtaggactttttaggccattgtaaagatgagcttttactctgagtaagacagagagctattagaggggtctcggtagaggagtgacttgattcaactgtctttgagaggatccctttgcgtgtatagactgtaaggtaacaggaataggccaagagagaatatttcggatgctagtgcaataatcaggtgagagatgatgaagactttgacctggataatagtagaaaaattgttgagaagggatcaaaatatggggtttgttttgatggtagagaggccagggatggctgaatgatcagatggggcatgagagagaaagaaaagaaacagagatgacttcaggaattttggcctgggccactggaaggatgaagtcaccatttactgagatggtaatgactgggaggttgagctggaagaactgagaatcaaatatctggttttgaatctgttctttttgagatgcatattcaacttccatatggaggtgtcaaggaggatttagatctagaattttggagctcaagggaaagggttgagctgtagacataaattctagagatgccggaatatagattgtgatccttctttatcagcacagaaatgacttgactttgtccaaactaagcaatcatactgtacatgttagcaacacattttacagggccaatttggccttttgcaatgttctgtggtttctaagataaataaacatattatatgtttccctctgga

SEQ ID NO: 6 - protein, 359 aa, Homo sapiens
MKATIILLLLAQVSWAGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 7 - DNA, 2761 bp, Homo sapiens
  source 1..2761: mol_type = genomic DNA
aaagaatttctcaggctcaaaatccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcagaaaacagatcttgattttttttctcttgaatgtacccttcaactgttgaatgtttaatagtaaatcttatatgtccttatttactttttagctttctctcaaataaagtgtaacactagttgagataacacatgaaagctctttaaagggtcgatcgggaacaggaaaaaaaacctatggaaaatatgacaacac

SEQ ID NO: 8 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 9 - DNA, 1175 bp, Homo sapiens
  source 1..1175: mol_type = genomic DNA
gagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 10 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 11 - DNA, 1479 bp, Homo sapiens
  source 1..1479: mol_type = genomic DNA
gttcagcatttctactccttccaagaagagcagcaaagctgaagtagcagcagcagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagagcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgcattatacattcatgcatttctaggctagagaaccttctagatttgatgcttacaactattctgttgtgactatgagaacatttctgtctctagaagtcatctgtctgtattgatctttatgctatattactatctgtggttacggtggagacattgacattattactggagtcaagcccttataagtcaaaagcatctatgtgtcgtaaaacattcctcaaacattttttcatgcaaatacacacttctttccccaaacatcatgtagcacatcaatatgtagggagacattcttatgcatcatttggtttgttttataaccaattcattaaatgtaattcataaaatgtactatgaaaaaaattatacgctatgggatactggcaaaagtgcacatatttcataaccaaattagtagcaccagtcttaatttgatgtttttcaacttttattcattgagatgttttgaagcaattaggatatgtgtgtttactgtactttttgttttgatccgtttgtataaatgatagcaatatcttggacacatctgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaa

SEQ ID NO: 12 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 13 - DNA, 773 bp, Homo sapiens
  source 1..773: mol_type = genomic DNA
attggccacagagacccagcccgagtttcccatcgcactgagcactgagatcctgctggaagctctgccgcagcatgagctccgcagccgggttctgcgcctcacgccccgggctgctgttcctggggttgctgctcctgccacttgtggtcgccttcgccagcgctgaagctgaagaagatggggacctgcagtgcctgtgtgtgaagaccacctcccaggtccgtcccaggcacatcaccagcctggaggtgatcaaggccggaccccactgccccactgcccaactgatagccacgctgaagaatggaaggaaaatttgcttggacctgcaagccccgctgtacaagaaaataattaagaaacttttggagagttagctactagctgcctacgtgtgtgcatttgctatatagcatacttcttttttccagtttcaatctaactgtgaaagaacttctgatatttgtgttatccttatgattttaaataaacaaaataaatcaagttgtagtatagtcaaaatacttcttaataatagtgcaaaaattgtgttgacacataacaatttcatggaagaaaaaaattccggtattttaagcaaaaagtattttgaaggaaggtgtgaatactggttatgcttggtgttacatgttggctgatacatattcatgcatttacatgattgcagtactttatagctacatatttaccttgaccattattattacctttgccaataaatatcagtaacacagatggcttttaaaaaa

SEQ ID NO: 14 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

(19) United States
(12) Patent Application Publication
(10) Pub. No.: US 2025/0241987 A1
(43) Pub. Date: Jul. 31, 2025

(54) COMPOSITIONS AND METHODS FOR PROMOTING WOUND HEALING AND MINIMIZING SCARRING

(71) Applicants: University of Pittsburgh - Of the Commonwealth System of Higher Education, Pittsburgh, PA (US); The United States Government as represented by the Department of Veterans Affairs, Washington, DC (US); Cornell University, Ithaca, NY (US)

(72) Inventors: Alan H. Wells, Pittsburgh, PA (US); Yadong Wang, Ithaca, NY (US)

(73) Assignees: University of Pittsburgh - Of the Commonwealth System of Higher Education, Pittsburgh, PA (US); The United States Government as represented by the Department of Veterans Affairs, Washington, DC (US); Cornell University, Ithaca, NY (US)

(21) Appl. No.: 18/848,224

(22) PCT Filed: Mar. 21, 2023
(86) PCT No.: PCT/US2023/064780

§ 371 Date: Sep. 18, 2024

Related U.S. Application Data
(60) Provisional application No. 63/322,964, filed on Mar. 23, 2022.

(51) Int. Cl. A61K 38/18; A61K 9/48; A61K 38/17; A61K 38/19; A61P 17/02

(52) U.S. Cl. CPC A61K 38/1808; A61K 9/4866; A61K 38/177; A61K 38/19; A61P 17/02

(57) ABSTRACT

Controlled delivery compositions and methods that enhance wound closure and reduce fibrotic scarring are described. The methods include treating a wound in a subject by administering to the subject a therapeutically effective amount of a first agent that promotes wound closure and a therapeutically effective amount of a second agent that inhibits scarring. The controlled delivery compositions include a therapeutically effective amount of a first agent that promotes wound closure and a therapeutically effective amount of a second agent that inhibits scarring. The controlled delivery compositions permit rapid release of the first agent and delayed release of the second agent.

### CROSS REFERENCE TO RELATED APPLICATIONS

This application claims the benefit of U.S. Provisional Application No. 63/322,964, filed Mar. 23, 2022, which is herein incorporated by reference in its entirety.

### ACKNOWLEDGMENT OF GOVERNMENT SUPPORT

This invention was made with government support under grant numbers NR016436 and GM063569 awarded by the National Institutes of Health, and Merit Award ONCA-019-18F awarded by the Department of Veterans Affairs. The government has certain rights in the invention.

### FIELD

This disclosure concerns controlled delivery methods and compositions that accelerate wound closure and reduce fibrotic scarring.

### INCORPORATION OF ELECTRONIC SEQUENCE LISTING

The electronic sequence listing, submitted herewith as an XML file named 8123-107870-02.xml (39,990 bytes), created on Mar. 16, 2023, is herein incorporated by reference in its entirety.

### BACKGROUND

Cutaneous wound healing is a dynamic and well-organized series of overlapping phases of repair that culminates in forming newly intact dermal and epidermal tissue layers. Pathological ailments such as diabetes and vascular disease obstruct the early phases of the wound healing process and result in the formation of a chronic wound. Wounds become considered chronic when they fail to close after three months post incident. One of the primary complications that lead to chronic wounds is disruptions to the blood supply. Rapid generation of new vasculature is necessary early within the wound healing process to deliver nutrients and oxygen, remove cellular and extracellular debris, and help with the transportation of cells into and out of the wound micro-environment.

### SUMMARY

Controlled delivery methods and compositions that enhance wound healing (such as wound closure) and reduce fibrotic scarring are described. Specifically provided are methods of treating a wound, such as a dermal wound or a mucosal wound, in a subject by administering to the subject a therapeutically effective amount of a first agent that promotes wound closure and a therapeutically effective amount of a second agent that inhibits scarring.

In some aspects, the first agent is administered to the subject prior to administration of the second agent, for example 1 day to 3 weeks prior to administration of the second agent.

In other aspects, the first agent and the second agent are administered concurrently in a controlled delivery composition that permits rapid release of the first agent and delayed release of the second agent. In some examples, the controlled delivery composition includes a hydrogel, and the hydrogel includes (i) the first agent, and (ii) the second agent which is encapsulated in a coacervate, such that upon administration of the controlled delivery composition, the first agent is released from the hydrogel at a faster rate than the second agent is released from the coacervate. In other examples, the controlled delivery composition includes a first coacervate that includes the first agent; a second coacervate that includes the second agent, wherein the second coacervate is encapsulated by lipids, thereby forming a lipo-coacervate, such that upon administration of the controlled delivery composition, the first agent is released from the first coacervate prior to release of the second agent from the lipo-coacervate. In other examples, the controlled delivery composition includes a first coacervate that includes the first agent, wherein the first coacervate is encapsulated by lipids, thereby forming a first lipo-coacervate; and a second coacervate that includes the second agent, wherein the second coacervate is encapsulated by lipids, thereby forming a second lipo-coacervate, such that upon administration of the controlled delivery composition, the first agent is released from the first lipo-coacervate prior to release of the second agent from the second lipo-coacervate.

Also provided are controlled delivery compositions for treating a wound that include a therapeutically effective amount of a first agent that promotes wound closure and a therapeutically effective amount of a second agent that inhibits scarring, such that the controlled delivery compositions permit rapid release of the first agent and delayed release of the second agent.

In some aspects, the controlled delivery composition includes a hydrogel, wherein the hydrogel includes the first agent and the second agent encapsulated in a coacervate, such that upon administration of the controlled delivery composition, the first agent is released from the hydrogel prior to release of the second agent from the coacervate.

In other aspects, the controlled delivery composition includes a first coacervate that includes the first agent, a second coacervate that includes the second agent, wherein the second coacervate is encapsulated by lipids, thereby forming a lipo-coacervate, such that upon administration of the controlled delivery composition, the first agent is released from the first coacervate prior to release of the second agent from the lipo-coacervate.

In other aspects, the controlled delivery composition includes a first coacervate that includes the first agent, wherein the first coacervate is encapsulated by lipids, thereby forming a first lipo-coacervate; and a second coacervate that includes the second agent, wherein the second coacervate is encapsulated by lipids, thereby forming a second lipo-coacervate, such that upon administration of the controlled delivery composition, the first agent is released from the first lipo-coacervate prior to release of the second agent from the second lipo-coacervate.

The foregoing and other features of this disclosure will become more apparent from the following detailed description of several aspects which proceeds with reference to the accompanying figures.

### BRIEF DESCRIPTION OF THE DRAWINGS

FIGS. 1A-1D: Tenascin-C (TNC) protects mesenchymal stem cells (MSCs) against ischemic conditions in vitro. MSCs were seeded onto either plastic, collagen type 1 (Col-1), or TNC+Col-1 coated plates and placed under hypoxia and nutrient deprivation (H/ND) conditions for up to 9 days. (FIG. 1A) Flow cytometry dot blot of MSCs stained with Annexin V and propidium iodide (PI) at day 7 H/ND where apoptosis begins to take effect. (FIG. 1B) Quantification of flow data normalized to counting beads expressed as cells negatively stained for Annexin and PI. (FIG. 1C) MSC live cell staining at day 7 for caspase 3/7 and (FIG. 1D) quantification using ImageJ software to find % positive caspase 3/7 staining. Quantification in FIG. 1C and FIG. 1D are shown as mean±SD. *p<0.05, **p<0.01, ***p<0.001. The scale bar for all images is 100 μm. Cytometry was performed three times, staining was performed three times, each in duplicate.

FIGS. 2A-2D: MSC-TNC conditioned media (CM) improves endothelial cell cord formation and motility. Bone marrow (BM)-MSCs were seeded onto either plastic, Col-1, or TNC+Col-1 coated plates and placed under H/ND conditions. (FIG. 2A) Cord assay was performed by collecting 96-hour CM and mixing with human dermal microvascular endothelial cells (HMEC-1) at 2×10⁵ cells per well onto growth factor reduced Matrigel in ibidi angiogenesis slides. Images were taken 6 hours post-seeding and quantified using ImageJ Angiogenesis Analyzer for (FIG. 2B) the total number of cords, total cord length, number of meshes, total mesh area. (FIG. 2C) CM was also used to determine the pro-migration effects on HMEC-1 across a denuded area with images being taken at 0- and 24-hour time points and (FIG. 2D) ImageJ quantification for % wound closure for each treatment group. Quantification in FIG. 2B and FIG. 2D are shown as mean±s.e.m. *p<0.05, **p<0.01, ***p<0.001. The scale bar for all images is 400 μm. The experiments were performed twice, each in triplicate.

FIGS. 3A-3C: Evaluation and comparison of pro-angiogenic factors for MSC paracrine activity. MSCs were seeded onto either plastic, Col-1, or TNC+Col-1 coated plates and placed under H/ND conditions. (FIG. 3A) CM was collected at 96 hours and analyzed using a Human Angiogenesis Array Kit and (FIG. 3B) quantified for relative expression compared to positive control spots. (FIG. 3C) Parallel run treatment plates were harvested at 48 hours for mRNA, processed into cDNA, and quantified using qPCR for expression differences in cytokine targets found in the CM. Gene expression profiles are displayed in terms of fold-regulation. Quantification in FIG. 3B is shown as mean±SD. *p<0.05, **p<0.01, ***p<0.001. All experiments were performed twice, in duplicate.

FIGS. 4A-4G: Assessment of the tissue replacement phase of CXCR3−/− mice treated with matricellular protein TNC and MSCs embedded in coacervate. Representative histological sections of Day 30 wounds stained with (FIG. 4A) H&E and (FIG. 4B) Masson's trichrome (MT) at 10× and 40× magnification. (FIG. 4C) Immunohistochemistry of CD31 at 20× magnification to assess capillary density and (FIG. 4D) quantification of capillary density (Capillaries/mm²) using 5 sample images per treatment group. (FIG. 4E) Quantification of wound healing score and wound thickness measurement for (FIG. 4F) dermal and (FIG. 4G) epidermal skin layers. Quantification in FIG. 4E is shown as mean±SD, whereas FIGS. 4D, 4F, and 4G are shown as mean±SEM. *p<0.05, **p<0.01, ***p<0.001. Scale bar for all images is 100 μm. There were four mice per group.

FIGS. 5A-5F: Assessment of collagen alignment and maturation in CXCR3^{-/-} mice treated with matricellular protein TNC and MSCs embedded in coacervate. (FIG. 5A) Brightfield images of picrosirius red (PSR) staining at 20× magnification. (FIG. 5B) Polarized light microscopy of picrosirius red-stained sections reveals the birefringence properties of collagen type 1 and collagen type III. (FIG. 5C) OrientationJ color survey of PSR polarized images pseudo-colored for orientation alignment. (FIG. 5D) Image J Quantification of PSR polarized images to assess collagen maturation as displayed by the Col-1/Col-III ratio shown as mean±SD (n=4). (FIG. 5E) Distribution of collagen fibrils relative to their degree of alignment is shown as mean±SD (n=4). (FIG. 5F) Quantification of collagen orientation displayed as percent coherency where a score of 1 is total alignment and 0 is complete isotropy; shown as mean±SD (n=4). *p<0.05, **p<0.01, ***p<0.001. Scale bar for all images is 100 μm. There were four mice per group.

FIG. 6: Confirmation of hypoxia in Biospherix culture system. MSCs were seeded onto either plastic, Col-1, or TNC+Col-1 coated plates and placed under H/ND conditions. A live cell hypoxia probe was used on parallel plated immortalized bone marrow mesenchymal stem cell (IHMSC) plates, one at 21% ambient oxygen levels and one at 1% hypoxic oxygen levels. Images were taken 6 hours post exposure to hypoxia conditions. This served as a back-up method for checking if the Biospherix instrumentation was working properly.

FIGS. 7A-7C: Coacervate delivery study of matricellular protein TNC and MSCs on delayed wound healing mouse model. (FIG. 7A) Overview schematic demonstrating the preparation of the coacervate-TNC+MSC delivery to an in vivo mouse skin wound model. (FIG. 7B) ELISA exhibited a % cumulative release of TNC from coacervate over 14 days. (FIG. 7C) Overview of mouse wound healing timeline for wounding procedure and harvesting. There were 5 treatment groups (coacervate (CO) only, CO+TNC, CO+MSC, TNC+MSC, or CO+TNC+MSC), with each group having 4 animals.

FIGS. 8A-8B: Time course comparison of culture treatments on MSC survival during H/ND in vitro. MSCs were seeded onto either plastic, Col-1, or TNC+Col-1 coated plates and placed under H/ND conditions for up to 9 days. (FIG. 8A) PI analysis to assess percent survival among the different MSC treatment groups. (FIG. 8B) Flow cytometry dot plot of MSCs stained with Annexin V and PI at days 5, 7, and 9 where MSCs start to undergo apoptosis. Quantification using ImageJ software to find % positive PI staining in FIG. 8A and is expressed as mean±SD. Two-way ANOVA was used to distinguish significance during the time course. *p<0.05, **p<0.01, ***p<0.001, ****p<0.0001. Cytometry was performed three times, staining was performed three times, each in duplicate.

FIGS. 9A-9C: RT-PCR analysis of MSCs on different seeding conditions during H/ND. MSCs were seeded onto either plastic, Col-1, or TNC+Col-1 coated plates and placed under H/ND growth conditions for 48 hours. mRNA was isolated and reverse transcribed to generate cDNA of each sample and run on a (FIG. 9A) human angiogenesis profiler array and a (FIG. 9B) human wound healing profiler array. Gene expression profiles are displayed in terms of fold-regulation. (FIG. 9C) Gene expression values larger than 2 or smaller than-2 are highlighted in the table.

FIGS. 10A-10C: Coacervate delivery study of matricellular proteins on hypertrophic mouse scarring model. (FIG. 10A) Overview schematic demonstrating the preparation of the coacervate-decorin delivery to an in vivo mouse skin wound model. ELISA exhibited % cumulative release of (FIG. 10B) decorin and (FIG. 10C) heparin-binding EGF-like growth factor (HB-EGF) from coacervate over 14 days.

FIGS. 11A-11H: Early wound healing phase assessment of CXCR3-mice treated with matricellular proteins embedded in coacervate. Representative histological sections of wounds stained with (FIG. 11A) H&E and (FIG. 11B) MT at 10× and 40× magnification. (FIG. 11C) Quantification of % wound closure obtained from ImageJ distance analysis on H&E and MT image sets between the leading edge of the epidermal tongues on each side of the eschar. (FIG. 11D) Immunofluorescent stain of Involucrin at 10× magnification for epidermal maturation evaluation. Quantification of (FIG. 11E) wound healing score and (FIG. 11F) wound thickness measurement for epidermal and dermal skin layers. (FIG. 11G) Immunohistochemistry of CD45 at 40× magnification for (FIG. 11H) inflammatory infiltrate quantification. Quantification in FIG. 11C and FIG. 11E are shown as mean±SD (n=4), whereas FIG. 11F and FIG. 11H are shown as mean±SEM (n=4). *p<0.05, **p<0.01, ***p<0.001. Scale bar for all images is 100 μm.

FIGS. 12A-12H: Assessment of the tissue replacement phase of CXCR3−/− mice treated with matricellular proteins embedded in coacervate. Representative histological sections of wounds stained with (FIG. 12A) H&E and (FIG. 12B) MT at 10× and 40× magnification. Quantification of (FIG. 12C) wound healing score and (FIG. 12D) wound thickness measurement for epidermal and dermal skin layers. (FIG. 12E) Immunohistochemistry of Collagen IV at 60× magnification to assess the formation of the basement membrane. (FIG. 12F) Immunofluorescent stain of Involucrin at 10× magnification for epidermal maturation evaluation. (FIG. 12G) Immunofluorescent stain of CD31 at 40× magnification with vasculature highlighted by white arrows. (FIG. 12H) Quantification of capillary density (capillaries/mm²). Quantification in FIG. 12C is shown as mean±SD (n≥5), whereas FIG. 12D and FIG. 12H are shown as mean±SEM (n≥5). *p<0.05, **p<0.01, ***p<0.001. Scale bar for all images is 100 μm.

FIGS. 13A-13J: Assessment of the resolution phase of CXCR3^{-/-} mice treated with matricellular proteins embedded in coacervate. Representative histological sections of wounds stained with (FIG. 13A) H&E and (FIG. 13B) MT at 10× and 40× magnification. Quantification of (FIG. 13C) wound healing score and (FIG. 13D) wound thickness measurement for epidermal and dermal skin layers. Quantification in FIG. 13C is shown as mean±SD (n=12), FIG. 13D is shown as mean±SEM (n=12). (FIG. 13E) Brightfield images of PSR staining at 20× magnification. (FIG. 13F) Polarized light microscopy of PSR-stained sections revealing the birefringence properties of collagen type 1 and collagen type III. (FIG. 13G) Image J Quantification of PSR polarized images to assess collagen maturation as displayed by the Col-1/Col-III ratio shown as mean±SD (n≥7). (FIG. 13H) OrientationJ color survey of PSR polarized images pseudo-colored for orientation alignment. (FIG. 13I) Distribution of collagen fibrils relative to their degree of alignment shown as mean±SD (n=5). (FIG. 13J) Quantification of collagen orientation displayed as percent coherency where a score of 1 is total alignment and 0 is complete isotropy; shown as mean±SD (n=10). *p<0.05, **p<0.01, ***p<0.001. Scale bar for all images is 100 μm.

FIG. 14: Illustration overview of wound healing score assessment for cutaneous wound healing. Epidermal and dermal skin layers are assessed independently and given a score on a scale of 0 to 4, depending on the current wound healing phase being observed. Epidermal maturation scoring goes as follows: 0=no epidermal migration, 1=partial epidermal migration, 2=complete epidermal migration, 3=partial keratinization and an intact basement membrane, 4=complete keratinization and now considered completely healed. Dermal maturation scoring is as follows: 0=no healing, 1=inflammatory infiltration, 2=granulation tissue present-fibroplasias and angiogenesis, 3=collagen deposition replacing granulation tissue>50%, 4=complete replacement of granulation tissue and now considered completely healed.

FIGS. 15A-15G: Additional staining to assess treatment groups on tissue replacement phase during wound healing. (FIG. 15A) Brightfield images of PSR staining at 20× magnification. (FIG. 15B) Polarized light microscopy of PSR-stained sections reveals the birefringence properties of collagen type 1 and collagen type III. (FIG. 15C) Image J Quantification of PSR polarized images to assess collagen content displayed by the Col-1/Col-III ratio shown as mean±SD (n≥5). (FIG. 15D) Quantification of collagen orientation displayed as percent coherency where a score of 1 is total alignment and 0 is complete isotropy; shown as mean±SD (n≥5). (FIG. 15E) Immunohistochemistry of collagen IV at 40× magnification as a supplemental assessment for vasculature. (FIG. 15F) Immunohistochemistry of CD45 at 60× magnification for (FIG. 15G) inflammatory infiltrate quantification (mean±SEM, n=4). *p<0.05, **p<0.01, ***p<0.001. Scale bar for all images is 100 μm.

FIGS. 16A-16D: Quantification of collagen fibrils among different matricellular treatment groups in the resolution phase of CXCR3^{-/-} mice. (FIG. 16A) Polarized light microscopy of picrosirius red-stained sections at ×100 magnification with collagen type 1 and collagen type III. (FIG. 16B) CT-FIRE extracted fibers highlighted by lines overlayed on the PSR image. Quantification of collagen fibril diameter (FIG. 16C) and length (FIG. 16D) shown as mean±SD (n=8). *p<0.05, **p<0.01, ***p<0.001. Scale bar for all images is 50 μm.

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

| SEQ ID NO: 1 is a nucleotide sequence encoding human TNC (GENBAN K™ Accession | |
|---|---|
| No. NM_002160.4). | |
| 1 | ggccacagcc tgcctactgt cacccgcctc tcccgcgcgc agatacacgc ccccgcctcc |
| 61 | gtgggcacaa aggcagcgct gctggggaac tcgggggaac gcgcacgtgg gaaccgccgc |
| 121 | agctccacac tccaggtact tcttccaagg acctaggtct ctcgcccatc ggaaagaaaa |
| 181 | taattctttc aagaagatca gggacaactg atttgaagtc tactctgtgc ttctaaatcc |
| 241 | ccaattctgc tgaaagtgag ataccctaga gccctagagc cccagcagca cccagccaaa |
| 301 | cccacctcca ccatgggggc catgactcag ctgttggcag gtgtctttct tgctttcctt |
| 361 | gccctcgcta ccgaaggtgg ggtcctcaag aaagtcatcc ggcacaagcg acagagtggg |
| 421 | gtgaacgcca ccctgccaga agagaaccag ccagtggtgt ttaaccacgt ttacaacatc |
| 481 | aagctgccag tgggatccca gtgttcggtg gatctggagt cagccagtgg ggagaaagac |
| 541 | ctggcaccgc cttcagagcc cagcgaaagc tttcaggagc acacagtgga tggggaaaac |
| 601 | cagattgtct tcacacatcg catcaacatc ccccgccggg cctgtggctg tgccgcagcc |
| 661 | cctgatgtta aggagctgct gagcagactg gaggagctgg agaacctggt gtcttccctg |
| 721 | agggagcaat gtactgcagg agcaggctgc tgtctccagc ctgccacagg ccgcttggac |
| 781 | accaggccct tctgtagcgg tcggggcaac ttcagcactg aaggatgtgg ctgtgtctgc |
| 841 | gaacctggct ggaaaggccc caactgctct gagcccgaat gtccaggcaa ctgtcacctt |
| 901 | cgaggccggt gcattgatgg gcagtgcatc tgtgacgacg gcttcacggg cgaggactgc |
| 961 | agccagctgg cttgccccag cgactgcaat gaccagggca agtgcgtaaa tggagtctgc |
| 1021 | atctgtttcg aaggctacgc cggggctgac tgcagccgtg aaatctgccc agtgccctgc |
| 1081 | agtgaggagc acggcacatg tgtagatggc ttgtgtgtgt gccacgatgg ctttgcaggc |
| 1141 | gatgactgca acaagcctct gtgtctcaac aattgctaca accgtggacg atgcgtggag |
| 1201 | aatgagtgcg tgtgtgatga gggtttcacg ggcgaagact gcagtgagct catctgcccc |
| 1261 | aatgactgct tcgaccgggg ccgctgcatc aatggcacct gctactgcga agaaggcttc |
| 1321 | acaggtgaag actgcgggaa acccacctgc ccacatgcct gccacaccca gggccggtgt |
| 1381 | gaggaggggc agtgtgtatg tgatgagggc tttgccggtg tggactgcag cgagaagagg |
| 1441 | tgtcctgctg actgtcacaa tcgtggccgc tgtgtagacg ggcggtgtga gtgtgatgat |
| 1501 | ggtttcactg gagctgactg tggggagctc aagtgtccca atggctgcag tggccatggc |
| 1561 | cgctgtgtca atgggcagtg tgtgtgtgat gagggctata ctggggagga ctgcagccag |
| 1621 | ctacggtgcc ccaatgactg tcacagtcgg ggccgctgtg tcgagggcaa atgtgtatgt |
| 1681 | gagcaaggct tcaagggcta tgactgcagt gacatgagct gccctaatga ctgtcaccag |
| 1741 | cacggccgct gtgtgaatgg catgtgtgtt tgtgatgacg gctacacagg ggaagactgc |
| 1801 | cgggatcgcc aatgccccag ggactgcagc aacaggggcc tctgtgtgga cggacagtgc |
| 1861 | gtctgtgagg acggcttcac cggccctgac tgtgcagaac tctcctgtcc aaatgactgc |
| 1921 | catggccagg gtcgctgtgt gaatgggcag tgcgtgtgcc atgaaggatt tatgggcaaa |
| 1981 | gactgcaagg agcaaagatg tcccagtgac tgtcatggcc agggccgctg cgtggacggc |
| 2041 | cagtgcatct gccacgaggg cttcacaggc ctggactgtg gccagcactc ctgccccagt |
| 2101 | gactgcaaca acttaggaca atgcgtctcg ggccgctgca tctgcaacga gggctacagc |
| 2161 | ggagaagact gctcagaggt gtctcctccc aaagacctcg ttgtgacaga agtgacggaa |
| 2221 | gagacggtca acctggcctg ggacaatgag atgcgggtca cagagtacct tgtcgtgtac |
| 2281 | acgcccaccc acgagggtgg tctggaaatg cagttccgtg tgcctgggga ccagacgtcc |
| 2341 | accatcatcc aggagctgga gcctggtgtg gagtacttta tccgtgtatt tgccatcctg |
| 2401 | gagaacaaga agagcattcc tgtcagcgcc agggtggcca cgtacttacc tgcacctgaa |
| 2461 | ggcctgaaat tcaagtccat caaggagaca tctgtggaag tggagtggga tcctctagac |
| 2521 | attgcttttg aaacctggga gatcatcttc cggaatatga ataaagaaga tgagggagag |
| 2581 | atcaccaaaa gcctgaggag gccagagacc tcttaccggc aaactggtct agctcctggg |
| 2641 | caagagtatg agatatctct gcacatagtg aaaaacaata cccggggccc tggcctgaag |
| 2701 | agggtgacca ccacacgctt ggatgccccc agccagatcg aggtgaaaga tgtcacagac |
| 2761 | accactgcct tgatcacctg gttcaagccc ctggctgaga tcgatggcat tgagctgacc |
| 2821 | tacggcatca aagacgtgcc aggagaccgt accaccatcg atctcacaga ggacgagaac |
| 2881 | cagtactcca tcgggaacct gaagcctgac actgagtacg aggtgtccct catctcccgc |
| 2941 | agaggtgaca tgtcaagcaa cccagccaaa gagaccttca caacaggcct cgatgctccc |
| 3001 | aggaatcttc gacgtgtttc ccagacagat aacagcatca ccctggaatg gaggaatggc |
| 3061 | aaggcagcta ttgacagtta cagaattaag tatgccccca tctctggagg ggaccacgct |
| 3121 | gaggttgatg ttccaaagag ccaacaagcc acaaccaaaa ccacactcac aggtctgagg |
| 3181 | ccgggaactg aatatgggat tggagtttct gctgtgaagg aagacaagga gagcaatcca |
| 3241 | gcgaccatca acgcagccac agagttggac acgcccaagg accttcaggt ttctgaaact |
| 3301 | gcagagacca gcctgaccct gctctggaag acaccgttgg ccaaatttga ccgctaccgc |
| 3361 | ctcaattaca gtctccccac aggccagtgg gtgggagtgc agcttccaag aaacaccact |
| 3421 | tcctatgtcc tgagaggcct ggaaccagga caggagtaca atgtcctcct gacagccgag |
| 3481 | aaaggcagac acaagagcaa gcccgcacgt gtgaaggcat ccactgaaca agcccctgag |
| 3541 | ctggaaaacc tcaccgtgac tgaggttggc tgggatggcc tcagactcaa ctggaccgca |
| 3601 | gctgaccagg cctatgagca ctttatcatt caggtgcagg aggccaacaa ggtggaggca |
| 3661 | gctcggaacc tcaccgtgcc tggcagcctt cgggctgtgg acataccggg cctcaaggct |
| 3721 | gctacgcctt atacagtctc catctatggg gtgatccagg gctatagaac accagtgctc |
| 3781 | tctgctgagg cctccacagg ggaaactccc aatttgggag aggtcgtggt ggccgaggtg |
| 3841 | ggctgggatg ccctcaaact caactggact gctccagaag gggcctatga gtactttttc |
| 3901 | attcaggtgc aggaggctga cacagtagag gcagcccaga acctcaccgt cccaggagga |
| 3961 | ctgaggtcca cagacctgcc tgggctcaaa gcagccactc attataccat caccatccgc |
| 4021 | ggggtcactc aggacttcag cacaacccct ctctctgttg aagtcttgac agaggaggtt |
| 4081 | ccagatatgg gaaacctcac agtgaccgag gttagctggg atgctctcag actgaactgg |
| 4141 | accacgccag atggaaccta tgaccagttt actattcagg tccaggaggc tgaccaggtg |
| 4201 | gaagaggctc acaatctcac ggttcctggc agcctgcgtt ccatggaaat cccaggcctc |
| 4261 | agggctggca ctccttacac agtcaccctg cacggcgagg tcaggggcca cagcactcga |
| 4321 | ccccttgctg tagaggtcgt cacagaggat ctcccacagc tgggagattt agccgtgtct |
| 4381 | gaggttggct gggatggcct cagactcaac tggaccgcag ctgacaatgc ctatgagcac |
| 4441 | tttgtcattc aggtgcagga ggtcaacaaa gtggaggcag cccagaacct cacgttgcct |
| 4501 | ggcagcctca gggctgtgga catcccgggc ctcgaggctg ccacgcctta tagagtctcc |
| 4561 | atctatgggg tgatccgggg ctatagaaca ccagtactct ctgctgaggc ctccacagcc |
| 4621 | aaagaacctg aaattggaaa cttaaatgtt tctgacataa ctcccgagag cttcaatctc |
| 4681 | tcctggatgg ctaccgatgg gatcttcgag acctttacca ttgaaattat tgattccaat |
| 4741 | aggttgctgg agactgtgga atataatatc tctggtgctg aacgaactgc ccatatctca |
| 4801 | gggctacccc ctagtactga ttttattgtc tacctctctg gacttgctcc cagcatccgg |
| 4861 | accaaaacca tcagtgccac agccacgaca gaggccctgc cccttctgga aaacctaacc |
| 4921 | atttccgaca ttaatcccta cgggttcaca gtttcctgga tggcatcgga gaatgccttt |
| 4981 | gacagctttc tagtaacggt ggtggattct gggaagctgc tggaccccca ggaattcaca |
| 5041 | ctttcaggaa cccagaggaa gctggagctt agaggcctca taactggcat tggctatgag |
| 5101 | gttatggtct ctggcttcac ccaagggcat caaaccaagc ccttgagggc tgagattgtt |
| 5161 | acagaagccg aaccggaagt tgacaacctt ctggtttcag atgccacccc agacggtttc |
| 5221 | cgtctgtcct ggacagctga tgaaggggtc ttcgacaatt ttgttctcaa aatcagagat |
| 5281 | accaaaaagc agtctgagcc actggaaata accctacttg cccccgaacg taccagggac |
| 5341 | ataacaggtc tcagagaggc tactgaatac gaaattgaac tctatggaat aagcaaagga |
| 5401 | aggcgatccc agacagtcag tgctatagca acaacagcca tgggctcccc aaaggaagtc |
| 5461 | attttctcag acatcactga aaattcggct actgtcagct ggagggcacc cacagcccaa |
| 5521 | gtggagagct tccggattac ctatgtgccc attacaggag gtacaccctc catggtaact |
| 5581 | gtggacggaa ccaagactca gaccaggctg gtgaaactca tacctggcgt ggagtacctt |
| 5641 | gtcagcatca tcgccatgaa gggctttgag gaaagtgaac ctgtctcagg gtcattcacc |
| 5701 | acagctctgg atggcccatc tggcctggtg acagccaaca tcactgactc agaagccttg |
| 5761 | gccaggtggc agccagccat tgccactgtg gacagttatg tcatctccta cacaggcgag |
| 5821 | aaagtgccag aaattacacg cacggtgtcc gggaacacag tggagtatgc tctgaccgac |
| 5881 | ctcgagcctg ccacggaata cacactgaga atctttgcag agaaagggcc ccagaagagc |
| 5941 | tcaaccatca ctgccaagtt cacaacagac ctcgattctc caagagactt gactgctact |
| 6001 | gaggttcagt cggaaactgc cctccttacc tggcgacccc cccgggcatc agtcaccggt |
| 6061 | tacctgctgg tctatgaatc agtggatggc acagtcaagg aagtcattgt gggtccagat |
| 6121 | accacctcct acagcctggc agacctgagc ccatccaccc actacacagc caagatccag |
| 6181 | gcactcaatg ggcccctgag gagcaatatg atccagacca tcttcaccac aattggactc |
| 6241 | ctgtacccct tccccaagga ctgctcccaa gcaatgctga atggagacac gacctctggc |
| 6301 | ctctacacca tttatctgaa tggtgataag gctgaggcgc tggaagtctt ctgtgacatg |
| 6361 | acctctgatg ggggtggatg gattgtgttc ctgagacgca aaaacggacg cgagaacttc |
| 6421 | taccaaaact ggaaggcata tgctgctgga tttggggacc gcagagaaga attctggctt |
| 6481 | gggctggaca acctgaacaa aatcacagcc caggggcagt acgagctccg ggtggacctg |
| 6541 | cgggaccatg gggagacagc ctttgctgtc tatgacaagt tcagcgtggg agatgccaag |
| 6601 | actcgctaca agctgaaggt ggaggggtac agtgggacag caggtgactc catggcctac |
| 6661 | cacaatggca gatccttctc cacctttgac aaggacacag attcagccat caccaactgt |
| 6721 | gctctgtcct acaaaggggc tttctggtac aggaactgtc accgtgtcaa cctgatgggg |
| 6781 | agatatgggg acaataacca cagtcagggc gttaactggt tccactggaa gggccacgaa |
| 6841 | cactcaatcc agtttgctga gatgaagctg agaccaagca acttcagaaa tcttgaaggc |
| 6901 | aggcgcaaac gggcataaat tccagggacc actgggtgag agaggaataa ggcccagagc |
| 6961 | gaggaaagga ttttaccaaa gcatcaatac aaccagccca accatcggtc cacacctggg |
| 7021 | catttggtga gagtcaaagc tgaccatgga tccctggggc caacggcaac agcatgggcc |
| 7081 | tcacctcctc tgtgatttct ttctttgcac caaagacatc agtctccaac atgtttctgt |
| 7141 | tttgttgttt gattcagcaa aaatctccca gtgacaacat cgcaatagtt ttttacttct |
| 7201 | cttaggtggc tctgggaatg ggagaggggt aggatgtaca ggggtagttt gttttagaac |
| 7261 | cagccgtatt ttacatgaag ctgtataatt aattgtcatt atttttgtta gcaaagatta |
| 7321 | aatgtgtcat tggaagccat cccttttttt acatttcata caacagaaac cagaaaagca |
| 7381 | atactgtttc cattttaagg atatgattaa tattattaat ataataatga tgatgatgat |
| 7441 | gatgaaaact aaggattttt caagagatct ttctttccaa aacatttctg gacagtacct |
| 7501 | gattgtattt tttttttaaa taaaagcaca agtacttttg agtttgttat tttgctttga |
| 7561 | attgttgagt ctgaatttca ccaaagccaa tcatttgaac aaagcgggga atgttgggat |
| 7621 | aggaaaggta agtagggata gtggtcaagt gggaggggtg gaaaggagac taaagactgg |
| 7681 | gagagaggga agcacttttt ttaaataaag ttgaacacac ttgggaaaag cttacaggcc |
| 7741 | aggcctgtaa tcccaacact ttgggaggcc aaggtgggag gatagcttaa ccccaggagt |
| 7801 | ttgagaccag cctgagcaac atagtgagaa cttgtctcta cagaaaaaaa aaaaaaaaaa |
| 7861 | aatttaatta ggcaagcgtg gtagtgcgca cctgtcgtcc cagctactca ggaggctgag |
| 7921 | gtaggaaaat cactggagcc caggagttag aggttacagt gagctatgat cacactactg |
| 7981 | cactccagcc tgggcaacag agggagaccc tgtctctaaa taaaaaaaga aaagaaaaaa |
| 8041 | aaagcttaca acttgagatt cagcatcttg ctcagtattt ccaagactaa tagattatgg |
| 8101 | tttaaaagat gcttttatac tcattttcta atgcaactcc tagaaactct atgatatagt |
| 8161 | tgaggtaagt attgttacca cacatgggct aagatcccca gaggcagact gcctgagttc |
| 8221 | aattcttggc tccaccattc ccaagttccc taacctctct atgcctcagt ttcctcttct |
| 8281 | gtaaagtagg gacactcata cttctcattt cagaacattt ttgtgaagaa taaattatgt |
| 8341 | tatccatttg aggcccttag aatggtaccc ggtgtatatt aagtgctagt acatgttagc |
| 8401 | tatcatcatt atcactttat atgagatgga ctggggttca tagaaaccca atgacttgat |
| 8461 | tgtggctact actcaataaa taatagaatt tggatttaaa |
| SEQ ID NO: 2 is an exemplary amino acid sequence of human TNC (GENBANK ™ | |
| Accession No. NP_002151.2). | |
| 1 | MGAMTQLLAG VFLAFLALAT EGGVLKKVIR HKRQSGVNAT LPEENQPVVF NHVYNIKLPV |
| 61 | GSQCSVDLES ASGEKDLAPP SEPSESFQEH TVDGENQIVF THRINIPRRA CGCAAAPDVK |
| 121 | ELLSRLEELE NLVSSLREQC TAGAGCCLQP ATGRLDTRPF CSGRGNFSTE GCGCVCEPGW |
| 181 | KGPNCSEPEC PGNCHLRGRC IDGQCICDDG FTGEDCSQLA CPSDCNDQGK CVNGVCICFE |
| 241 | GYAGADCSRE ICPVPCSEEH GTCVDGLCVC HDGFAGDDCN KPLCLNNCYN RGRCVENECV |
| 301 | CDEGFTGEDC SELICPNDCF DRGRCINGTC YCEEGFTGED CGKPTCPHAC HTQGRCEEGQ |
| 361 | CVCDEGFAGV DCSEKRCPAD CHNRGRCVDG RCECDDGFTG ADCGELKCPN GCSGHGRCVN |
| 421 | GQCVCDEGYT GEDCSQLRCP NDCHSRGRCV EGKCVCEQGF KGYDCSDMSC PNDCHQHGRC |
| 481 | VNGMCVCDDG YTGEDCRDRQ CPRDCSNRGL CVDGQCVCED GFTGPDCAEL SCPNDCHGQG |
| 541 | RCVNGQCVCH EGFMGKDCKE QRCPSDCHGQ GRCVDGQCIC HEGFTGLDCG QHSCPSDCNN |
| 601 | LGQCVSGRCI CNEGYSGEDC SEVSPPKDLV VTEVTEETVN LAWDNEMRVT EYLVVYTPTH |
| 661 | EGGLEMQFRV PGDQTSTIIQ ELEPGVEYFI RVFAILENKK SIPVSARVAT YLPAPEGLKF |
| 721 | KSIKETSVEV EWDPLDIAFE TWEIIFRNMN KEDEGEITKS LRRPETSYRQ TGLAPGQEYE |
| 781 | ISLHIVKNNT RGPGLKRVTT TRLDAPSQIE VKDVTDTTAL ITWFKPLAEI DGIELTYGIK |
| 841 | DVPGDRTTID LTEDENQYSI GNLKPDTEYE VSLISRRGDM SSNPAKETFT TGLDAPRNLR |
| 901 | RVSQTDNSIT LEWRNGKAAI DSYRIKYAPI SGGDHAEVDV PKSQQATTKT TLTGLRPGTE |
| 961 | YGIGVSAVKE DKESNPATIN AATELDTPKD LQVSETAETS LILLWKTPLA KFDRYRLNYS |
| 1021 | LPTGQWVGVQ LPRNTTSYVL RGLEPGQEYN VLLTAEKGRH KSKPARVKAS TEQAPELENL |
| 1081 | TVTEVGWDGL RLNWTAADQA YEHFIIQVQE ANKVEAARNL TVPGSLRAVD IPGLKAATPY |
| 1141 | TVSIYGVIQG YRTPVLSAEA STGETPNLGE VVVAEVGWDA LKLNWTAPEG AYEYFFIQVQ |
| 1201 | EADTVEAAQN LTVPGGLRST DLPGLKAATH YTITIRGVTQ DFSTTPLSVE VLTEEVPDMG |
| 1261 | NLTVTEVSWD ALRLNWTTPD GTYDQFTIQV QEADQVEEAH NLTVPGSLRS MEIPGLRAGT |
| 1321 | PYTVTLHGEV RGHSTRPLAV EVVTEDLPQL GDLAVSEVGW DGLRLNWTAA DNAYEHFVIQ |
| 1381 | VQEVNKVEAA QNLTLPGSLR AVDIPGLEAA TPYRVSIYGV IRGYRTPVLS AEASTAKEPE |
| 1441 | IGNLNVSDIT PESENLSWMA TDGIFETETI ELIDSNRLLE TVEYNISGAE RTAHISGLPP |
| 1501 | STDFIVYLSG LAPSIRTKTI SATATTEALP LLENLTISDI NPYGFTVSWM ASENAFDSEL |
| 1561 | VTVVDSGKLL DPQEFTLSGT QRKLELRGLI TGIGYEVMVS GFTQGHQTKP LRAEIVTEAE |
| 1621 | PEVDNLLVSD ATPDGFRLSW TADEGVFDNF VLKIRDTKKQ SEPLEITLLA PERTRDITGL |
| 1681 | REATEYEIEL YGISKGRRSQ TVSAIATTAM GSPKEVIFSD ITENSATVSW RAPTAQVESE |
| 1741 | RITYVPITGG TPSMVTVDGT KTQTRLVKLI PGVEYLVSII AMKGFEESEP VSGSFTTALD |
| 1801 | GPSGLVTANI TDSEALARWQ PAIATVDSYV ISYTGEKVPE ITRTVSGNTV EYALTDLEPA |
| 1861 | TEYTLRIFAE KGPQKSSTIT AKFTTDLDSP RDLTATEVQS ETALLTWRPP RASVTGYLLV |
| 1921 | YESVDGTVKE VIVGPDTTSY SLADLSPSTH YTAKIQALNG PLRSNMIQTI FTTIGLLYPF |
| 1981 | PKDCSQAMLN GDTTSGLYTI YLNGDKAEAL EVFCDMTSDG GGWIVELRRK NGRENFYQNW |
| 2041 | KAYAAGFGDR REEFWLGLDN LNKITAQGQY ELRVDLRDHG ETAFAVYDKF SVGDAKTRYK |
| 2101 | LKVEGYSGTA GDSMAYHNGR SFSTEDKDTD SAITNCALSY KGAFWYRNCH RVNLMGRYGD |
| 2161 | NNHSQGVNWF HWKGHEHSIQ FAEMKLRPSN FRNLEGRRKR A |
| SEQ ID NO: 3 is a nucleotide sequence encoding human HB-EGF (GENBANK ™ | |
| Accession No. NM_001945.3). | |
| 1 | attcggccga aggagctacg cgggccacgc tgctggctgg cctgacctag gcgcgcgggg |
| 61 | tcgggcggcc gcgcgggcgg gctgagtgag caagacaaga cactcaagaa gagcgagctg |
| 121 | cgcctgggtc ccggccaggc ttgcacgcag aggcgggcgg cagacggtgc ccggcggaat |
| 181 | ctcctgagct ccgccgccca gctctggtgc cagcgcccag tggccgccgc ttcgaaagtg |
| 241 | actggtgcct cgccgcctcc tctcggtgcg ggaccatgaa gctgctgccg tcggtggtgc |
| 301 | tgaagctctt tctggctgca gttctctcgg cactggtgac tggcgagagc ctggagcggc |
| 361 | ttcggagagg gctagctgct ggaaccagca acccggaccc tcccactgta tccacggacc |
| 421 | agctgctacc cctaggaggc ggccgggacc ggaaagtccg tgacttgcaa gaggcagatc |
| 481 | tggacctttt gagagtcact ttatcctcca agccacaagc actggccaca ccaaacaagg |
| 541 | aggagcacgg gaaaagaaag aagaaaggca aggggctagg gaagaagagg gacccatgtc |
| 601 | ttcggaaata caaggacttc tgcatccatg gagaatgcaa atatgtgaag gagctccggg |
| 661 | ctccctcctg catctgccac ccgggttacc atggagagag gtgtcatggg ctgagcctcc |
| 721 | cagtggaaaa tcgcttatat acctatgacc acacaaccat cctggccgtg gtggctgtgg |
| 781 | tgctgtcatc tgtctgtctg ctggtcatcg tggggcttct catgtttagg taccatagga |
| 841 | gaggaggtta tgatgtggaa aatgaagaga aagtgaagtt gggcatgact aattcccact |
| 901 | gagagagact tgtgctcaag gaatcggctg gggactgcta cctctgagaa gacacaaggt |
| 961 | gatttcagac tgcagagggg aaagacttcc atctagtcac aaagactcct tcgtccccag |
| 1021 | ttgccgtcta ggattgggcc tcccataatt gctttgccaa aataccagag ccttcaagtg |
| 1081 | ccaaacagag tatgtccgat ggtatctggg taagaagaaa gcaaaagcaa gggaccttca |
| 1141 | tgcccttctg attcccctcc accaaacccc acttcccctc ataagtttgt ttaaacactt |
| 1201 | atcttctgga ttagaatgcc ggttaaattc catatgctcc aggatctttg actgaaaaaa |
| 1261 | aaaaagaaga agaagaagga gagcaagaag gaaagatttg tgaactggaa gaaagcaaca |
| 1321 | aagattgaga agccatgtac tcaagtacca ccaagggatc tgccattggg accctccagt |
| 1381 | gctggatttg atgagttaac tgtgaaatac cacaagcctg agaactgaat tttgggactt |
| 1441 | ctacccagat ggaaaaataa caactatttt tgttgttgtt gtttgtaaat gcctcttaaa |
| 1501 | ttatatattt attttattct atgtatgtta atttatttag tttttaacaa tctaacaata |
| 1561 | atatttcaag tgcctagact gttactttgg caatttcctg gccctccact cctcatcccc |
| 1621 | acaatctggc ttagtgccac ccacctttgc cacaaagcta ggatggttct gtgacccatc |
| 1681 | tgtagtaatt tattgtctgt ctacatttct gcagatcttc cgtggtcaga gtgccactgc |
| 1741 | gggagctctg tatggtcagg atgtaggggt taacttggtc agagccactc tatgagttgg |
| 1801 | acttcagtct tgcctaggcg attttgtcta ccatttgtgt tttgaaagcc caaggtgctg |
| 1861 | atgtcaaagt gtaacagata tcagtgtctc cccgtgtcct ctccctgcca agtctcagaa |
| 1921 | gaggttgggc ttccatgcct gtagctttcc tggtccctca cccccatggc cccaggccca |
| 1981 | cagcgtggga actcactttc ccttgtgtca agacatttct ctaactcctg ccattcttct |
| 2041 | ggtgctactc catgcagggg tcagtgcagc agaggacagt ctggagaagg tattagcaaa |
| 2101 | gcaaaaggct gagaaggaac agggaacatt ggagctgact gttcttggta actgattacc |
| 2161 | tgccaattgc taccgagaag gttggaggtg gggaaggctt tgtataatcc cacccacctc |
| 2221 | accaaaacga tgaagttatg ctgtcatggt cctttctgga agtttctggt gccatttctg |
| 2281 | aactgttaca acttgtattt ccaaacctgg ttcatattta tactttgcaa tccaaataaa |
| 2341 | gataaccctt attccata |
| SEQ ID NO: 4 is an exemplary amino acid sequence of human HB-EGF (GENBANK ™ | |
| Accession No. NP_001936.1) | |
| 1 | MKLLPSVVLK LFLAAVLSAL VTGESLERLR RGLAAGTSNP DPPTVSTDQL LPLGGGRDRK |
| 61 | VRDLQEADLD LLRVILSSKP QALATPNKEE HGKRKKKGKG LGKKRDPCLR KYKDFCIHGE |
| 121 | CKYVKELRAP SCICHPGYHG ERCHGLSLPV ENRLYTYDHT TILAVVAVVL SSVCLLVIVG |
| 181 | LLMFRYHRRG GYDVENEEKV KLGMTNSH |
| SEQ ID NO: 5 is a nucleotide sequence encoding human decorin (GENBANK ™ | |
| Accession No. NM_001920.5). | |
| 1 | aactgtgcta tggagtagaa gcaggaggtt ttcaacctag tcacagagca gcacctaccc |
| 61 | cctcctcctt tccacacctg caaactcttt tacttgggct gaatatttag tgtaattaca |
| 121 | tctcagcttt gagggctcct gtggcaaatt cccggattaa aaggttccct ggttgtgaaa |
| 181 | atacatgaga taaatcatga aggccactat catcctectt ctgcttgcac aagtttcctg |
| 241 | ggctggaccg tttcaacaga gaggcttatt tgactttatg ctagaagatg aggcttctgg |
| 301 | gataggccca gaagttcctg atgaccgcga cttcgagccc tocctaggcc cagtgtgccc |
| 361 | cttccgctgt caatgccatc ttcgagtggt ccagtgttct gatttgggtc tggacaaagt |
| 421 | gccaaaggat cttccccctg acacaactct gctagacctg caaaacaaca aaataaccga |
| 481 | aatcaaagat ggagacttta agaacctgaa gaaccttcac gcattgatto ttgtcaacaa |
| 541 | taaaattage aaagttagtc ctggagcatt tacacctttg gtgaagttgg aacgacttta |
| 601 | tctgtccaag aatcagctga aggaattgcc agaaaaaatg cccaaaactc ttcaggagct |
| 661 | gcgtgcccat gagaatgaga tcaccaaagt gcgaaaagtt actttcaatg gactgaacca |
| 721 | gatgattgtc atagaactgg gcaccaatcc gctgaagagc tcaggaattg aaaatggggc |
| 781 | tttccaggga atgaagaagc tctcctacat ccgcattgct gataccaata tcaccagcat |
| 841 | tcctcaaggt cttcctcctt cccttacgga attacatctt gatggcaaca aaatcagcag |
| 901 | agttgatgca gctagcctga aaggactgaa taatttggct aagttgggat tgagtttcaa |
| 961 | cagcatctct gctgttgaca atggctctct ggccaacacg cctcatctga gggagcttca |
| 1021 | cttggacaac aacaagctta ccagagtacc tggtgggctg gcagagcata agtacatcca |
| 1081 | ggttgtctac cttcataaca acaatatctc tgtagttgga tcaagtgact tctgcccacc |
| 1141 | tggacacaac accaaaaagg cttcttattc gggtgtgagt cttttcagca acccggtcca |
| 1201 | gtactgggag atacagccat ccaccttcag atgtgtctac gtgcgctctg ccattcaact |
| 1261 | cggaaactat aagtaattct caagaaagcc ctcattttta taacctggca aaatcttgtt |
| 1321 | aatgtcattg ctaaaaaata aataaaagct agatactgga aacctaactg caatgtggat |
| 1381 | gttttaccca catgacttat tatgcataaa gccaaatttc cagtttaagt aattgcctac |
| 1441 | aataaaaaga aattttgcct gccattttca gaatcatctt ttgaagcttt ctgttgatgt |
| 1501 | taactgagct actagagata ttcttatttc actaaatgta aaatttggag taaatatata |
| 1561 | tgtcaatatt tagtaaagct tttctttttt aatttccagg aaaaaataaa aagagtatga |
| 1621 | gtcttctgta attcattgag cagttagctc atttgagata aagtcaaatg ccaaacacta |
| 1681 | gctctgtatt aatccccatc attactggta aagcctcatt tgaatgtgtg aattcaatac |
| 1741 | aggctatgta aaatttttac taatgtcatt attttgaaaa aataaattta aaaatacatt |
| 1801 | caaaattact attgtataca agcttaattg ttaatattcc ctaaacacaa ttttatgaag |
| 1861 | ggagaagaca ttggtttgtt gacaataaca gtacatettt tcaagttctc agctatttct |
| 1921 | tctacctctc cctatcttac atttgagtat ggtaacttat gtcatctatg ttgaatgtaa |
| 1981 | gcttataaag cacaaagcat acatttcctg actggtctag agaactgatg tttcaattta |
| 2041 | cccctctgct aaataaatat taaaactatc atgtgacttc atgtaatcag gctgaacatt |
| 2101 | tctacaatta ctagatgtat tagacgtaag tattttcttt agttaaacca cccatgttag |
| 2161 | aaatgttttc tgtagaattt ataaacaact atcaatgcag acaatttaat aagcctgggg |
| 2221 | atgatttact tacagtaaac atttatcaaa ttgtacattt gtgctatcaa caattaataa |
| 2281 | gcaaatatgt gaaaatagtt tctgtcttct atgaagttag atatttgatg gttaaaaccc |
| 2341 | ctataaatca tagtttcata tgggaaaaaa taattgaaat acagtgtaaa tttaaataat |
| 2401 | ttattaagta tagcaaataa ttgaaatatg gtggactaaa ttttgtcata gaaatatgtg |
| 2461 | caagttatag tagtggctca catgagaggt aatcaattct gctaatagta gcagaatgag |
| 2521 | tgcagtggaa catgaaaaac ttgaggagat aacagttgag gtgggtttcc atagatgcat |
| 2581 | aatagttcaa gagcaagatt tggtggggag gcactattca agacagggac taagttcaaa |
| 2641 | atccaagacg tatgctggga cacacctctg acaggttggc ataaaggagg cttaatcaaa |
| 2701 | ctatttttct tottctgaaa cagaagcaat aattttcatt tacatttgac atatcccgag |
| 2761 | gtaatattaa cattagggaa agttactctt ttccatcttt ccacattctt gcaggaccat |
| 2821 | aaaatctgaa ttttccagta tttttaataa gagggaagaa acctctcttt ttcttctctt |
| 2881 | tttcatctcc caagagatcc tcctctcatg actacagttg aataggtggt ttctattgga |
| 2941 | agacattcag gaattcaagg tgcatgtcca taaatggact ttttttgttg ttgttcagag |
| 3001 | ctggaccttg aatgatgcat ccttctctct gttgtaacca tgaataatgc acccttcatg |
| 3061 | ctatagcctt taacgattca cccttcttat tgtaaccttg aatgattcac cctttatggt |
| 3121 | gtagccttga gtgacgcacc cttcatgttg tagccttcaa tgatgcacac tccatgttat |
| 3181 | agccttgaat gatataccct ttatgctgca gcctttctct tatggggaaa agcctgcaga |
| 3241 | tatcctgctg cttaactgac aagtgtggtg agaaataagt agaaatctaa agaggggaag |
| 3301 | accattttgg acacttatct gcaaggcaga tocaacacac ttttccagta gtcaagctac |
| 3361 | ttctaatttt gttcagtatc aaaatgagaa acaggcctga ttctccagca ctcttgtcaa |
| 3421 | cacaacttcc ccccatattt atatatatac acacacatat atatctttat atatatacac |
| 3481 | acatatatat ctttatatat atatatttat atatatatct ttttgcatat atacatatat |
| 3541 | atgtatottt atttectttg aaataaagat aaatatagct gatttctttg gcttcgacac |
| 3601 | ttactatttg catgactaag ggaagctagt taacctttct gtgactcatt tccttgtcca |
| 3661 | taaaatggga atattaattg tacatgtctt atggattggt gtgtgaattc agttagcgag |
| 3721 | tgtagaatat aacttataga tcaaagtaga gtaaatggaa agggctcaac tatggtgttg |
| 3781 | ctactgccat tgttattaca ggcacacagt togagctata atcatttcaa gggaaattct |
| 3841 | tatgtgtcag ttctggatcg aggtctgaga ttctgcattt caaacaaact tccaggaatg |
| 3901 | ctgctgcttc ttggtccaca cttggagaaa taagtcagca gagagtcctc tcgtttccta |
| 3961 | ttgtaccatg tctgtctttt gtctcctgct tattggcctc tgtaaggaac tcacagctgc |
| 4021 | tataataaag taccaaaaac tgggtggctt aaaacaacag aaacttactt tctcacaatt |
| 4081 | ctggaggcta aaaattcaaa atcaaggtgt cagcagggte agattcette caaagccttt |
| 4141 | aggagaggac ctttccttgc tcctcctagg tttctggtta aatctaggtg ttctttgcct |
| 4201 | tatggcagcg tgactctaat ttatgcctcc atcttcaccc tcacatggcc ttctctotca |
| 4261 | tgtgtttgtg tcttttctct tottcttcta tgaacatgag ttatattgga ttaaggctca |
| 4321 | ccctaattta gtatgaaccc atcttaactt gattatatct gcaaagactc tatttccaaa |
| 4381 | tgaagtcaga ctcacaggta ttgggggttt gatattgaac atatcttctg ggaggagaca |
| 4441 | caacttaatc attaatatcc actttctttt ttccttatta aatgtttaat ttttttgttt |
| 4501 | tcattaaaac tgttgttcga ttatgggtgc ttcacataaa aggttggaaa cttaaaaatt |
| 4561 | tgtctctgac ccctcctggt tggaaaggcc tctgttgtac atttatgcta gcctaggcca |
| 4621 | taccactttc tgtcttcagt acagccatct tagtttattc aagtgacaca gattttccag |
| 4681 | aacacagtat tcatgatott ttaaagcatt tttcttcaaa gactttgatc tggcaataaa |
| 4741 | tgttactatg taattctcat gacataaatt aggcataact tggatctcct cttcttctgc |
| 4801 | tcattcattt gtctgaatca tcactattat cttttttata ttcctttacc gttctccata |
| 4861 | atgcttttcc aagaaactgg tttattccac aattttattg cagaggcagc tgcaggatat |
| 4921 | cataatctta totttatacg aaggaagaat tgcctaatcc ctcaagtaaa ctaaaaatgt |
| 4981 | tttatacagt catttctcat tcatccaatg ttccaggcag toccagtcca agactgcccc |
| 5041 | ttcacacaca caacaactct tcacaagact tcactgtect tcagactctc ctgcagcaca |
| 5101 | gacattcgag gttgctgagt cgacattgca gttatttcac tottgatttt gctgctcaat |
| 5161 | tttaagtttc cacacttatt gctaaaacat tocttctagt ggatctgttt atgtattcaa |
| 5221 | gcacaccatc tgtcacaatt atttcaaatc acaggtctgt gacagactgt caatttcaca |
| 5281 | ccacaaagta taatagtgga aaacaaagta aaatcaagaa agggagaaca ctaaacttat |
| 5341 | taaaaccata tacaagcata cgcattcagg aaactaaaac aaccttcaaa gaagttgaac |
| 5401 | aattttgagc caaaagaaag ggcatatgca caccctaccc cagtataaga taaccagaaa |
| 5461 | aggactgggt gagttgcctc ctgagtatta tgactgccat caatcactta ggcattggag |
| 5521 | atcaggagac tottcccagg ccacacattc tgcagtgact gtgagggctg aatgaatgct |
| 5581 | tccaggccat caggaacaag catagaaaca caaatcaatt ttaaaataca ctcaaaaatg |
| 5641 | tcaaaataat ccaacttttt tgtcttgate agatatatct actagcacat attacttcat |
| 5701 | tttttcagcc aatatttatt aataacctac tgtgtgcttg acagtgttct agtggcagag |
| 5761 | acacagcagt gcataatgcc ttgccctctg aagcagatgt tgcagcagga ggagacattc |
| 5821 | caggacatga caattaaagt ggaggaatgt caagcagaac tggggaataa gggatgtgaa |
| 5881 | gggggtgaga cttgctattt tatataggat ggtoggaaaa aggacttatc aggagtgaca |
| 5941 | tttgagcagg gagatgaaga actgaaggga gcaagccaag aaaagaggcc ctgaatttgg |
| 6001 | aaaatacatg gcacgttcca acaagagcag agaggacaaa cgtgagcaga gtaaagtgag |
| 6061 | caagaagaga taatatatga tacaaggtca gaaaggtaat gacaatcgca tcctgtagga |
| 6121 | ctttttaggc cattgtaaag atgagctttt actctgagta agacagagag ctattagagg |
| 6181 | ggtctcggta gaggagtgac ttgattcaac tgtctttgag aggatccctt tgcgtgtata |
| 6241 | gactgtaagg taacaggaat aggccaagag agaatatttc ggatgctagt gcaataatca |
| 6301 | ggtgagagat gatgaagact ttgacctgga taatagtaga aaaattgttg agaagggatc |
| 6361 | aaaatatggg gtttgttttg atggtagaga ggccagggat ggctgaatga tcagatgggg |
| 6421 | catgagagag aaagaaaaga aacagagatg acttcaggaa ttttggcctg ggccactgga |
| 6481 | aggatgaagt caccatttac tgagatggta atgactggga ggttgagctg gaagaactga |
| 6541 | gaatcaaata tctggttttg aatctgttct ttttgagatg catattcaac ttccatatgg |
| 6601 | aggtgtcaag gaggatttag atctagaatt ttggagctca agggaaaggg ttgagctgta |
| 6661 | gacataaatt ctagagatgc cggaatatag attgtgatcc ttctttatca gcacagaaat |
| 6721 | gacttgactt tgtccaaact aagcaatcat actgtacatg ttagcaacac attttacagg |
| 6781 | gccaatttgg ccttttgcaa tgttctgtgg tttctaagat aaataaacat attatatgtt |
| 6841 | tccctctgga |
| SEQ ID NO: 6 is an exemplary amino acid sequence of human decorin (GENBANK ™ | |
| Accession No. NP_001911.1) | |
| 1 | MKATIILLLL AQVSWAGPFQ QRGLFDFMLE DEASGIGPEV PDDRDFEPSL GPVCPFRCQC |
| 61 | HLRVVQCSDL GLDKVPKDLP PDTTLLDLQN NKITEIKDGD FKNLKNLHAL ILVNNKISKV |
| 121 | SPGAFTPLVK LERLYLSKNQ LKELPEKMPK TLQELRAHEN EITKVRKVTF NGLNQMIVIE |
| 181 | LGTNPLKSSG IENGAFQGMK KLSYIRIADT NITSIPQGLP PSLTELHLDG NKISRVDAAS |
| 241 | LKGLNNLAKL GLSFNSISAV DNGSLANTPH LRELHLDNNK LTRVPGGLAE HKYIQVVYLH |
| 301 | NNNISVVGSS DFCPPGHNTK KASYSGVSLF SNPVQYWEIQ PSTFRCVYVR SAIQLGNYK |
| SEQ ID NO: 7 is a nucleotide sequence encoding human CXCL9 (GENBANK ™ Accession | |
| No. NM_002416). | |
| 1 | aaagaatttc tcaggctcaa aatccaatac aggagtgact tggaactcca ttctatcact |
| 61 | atgaagaaaa gtggtgttct tttcctcttg ggcatcatct tgctggttct gattggagtg |
| 121 | caaggaaccc cagtagtgag aaagggtcgc tgttcctgca tcagcaccaa ccaagggact |
| 181 | atccacctac aatccttgaa agaccttaaa caatttgccc caagcccttc ctgcgagaaa |
| 241 | attgaaatca ttgctacact gaagaatgga gttcaaacat gtctaaaccc agattcagca |
| 301 | gatgtgaagg aactgattaa aaagtgggag aaacaggtca gccaaaagaa aaagcaaaag |
| 361 | aatgggaaaa aacatcaaaa aaagaaagtt ctgaaagttc gaaaatctca acgttctcgt |
| 421 | caaaagaaga ctacataaga gaccacttca ccaataagta ttctgtgtta aaaatgttct |
| 481 | attttaatta taccgctatc attccaaagg aggatggcat ataatacaaa ggcttattaa |
| 541 | tttgactaga aaatttaaaa cattactctg aaattgtaac taaagttaga aagttgattt |
| 601 | taagaatcca aacgttaaga attgttaaag gctatgattg tctttgttct tctaccaccc |
| 661 | accagttgaa tttcatcatg cttaaggcca tgattttagc aatacccatg tctacacaga |
| 721 | tgttcaccca accacatccc actcacaaca gctgcctgga agagcagccc taggcttcca |
| 781 | cgtactgcag cctccagaga gtatctgagg cacatgtcag caagtcctaa gcctgttagc |
| 841 | atgctggtga gccaagcagt ttgaaattga gctggacctc accaagctgc tgtggccatc |
| 901 | aacctctgta tttgaatcag cctacaggcc tcacacacaa tgtgtctgag agattcatgc |
| 961 | tgattgttat tgggtatcac cactggagat caccagtgtg tggctttcag agcctccttt |
| 1021 | ctggctttgg aagccatgtg attccatctt gcccgctcag gctgaccact ttatttcttt |
| 1081 | ttgttcccct ttgcttcatt caagtcagct cttctccatc ctaccacaat gcagtgcctt |
| 1141 | tcttctctcc agtgcacctg tcatatgctc tgatttatct gagtcaactc ctttctcatc |
| 1201 | ttgtccccaa caccccacag aagtgctttc ttctcccaat tcatcctcac tcagtccagc |
| 1261 | ttagttcaag tcctgcctct taaataaacc tttttggaca cacaaattat cttaaaactc |
| 1321 | ctgtttcact tggttcagta ccacatgggt gaacactcaa tggttaacta attcttgggt |
| 1381 | gtttatccta tctctccaac cagattgtca gctccttgag ggcaagagcc acagtatatt |
| 1441 | tccctgtttc ttccacagtg cctaataata ctgtggaact aggttttaat aattttttaa |
| 1501 | ttgatgttgt tatgggcagg atggcaacca gaccattgtc tcagagcagg tgctggctct |
| 1561 | ttcctggcta ctccatgttg gctagcctct ggtaacctct tacttattat cttcaggaca |
| 1621 | ctcactacag ggaccaggga tgatgcaaca tccttgtctt tttatgacag gatgtttgct |
| 1681 | cagcttctcc aacaataaga agcacgtggt aaaacacttg cggatattct ggactgtttt |
| 1741 | taaaaaatat acagtttacc gaaaatcata taatcttaca atgaaaagga ctttatagat |
| 1801 | cagccagtga ccaacctttt cccaaccata caaaaattcc ttttcccgaa ggaaaagggc |
| 1861 | tttctcaata agcctcagct ttctaagatc taacaagata gccaccgaga tccttatcga |
| 1921 | aactcatttt aggcaaatat gagttttatt gtccgtttac ttgtttcaga gtttgtattg |
| 1981 | tgattatcaa ttaccacacc atctcccatg aagaaaggga acggtgaagt actaagcgct |
| 2041 | agaggaagca gccaagtcgg ttagtggaag catgattggt gcccagttag cctctgcagg |
| 2101 | atgtggaaac ctccttccag gggaggttca gtgaattgtg taggagaggt tgtctgtggc |
| 2161 | cagaatttaa acctatactc actttcccaa attgaatcac tgctcacact gctgatgatt |
| 2221 | tagagtgctg tccggtggag atcccacccg aacgtcttat ctaatcatga aactccctag |
| 2281 | ttccttcatg taacttccct gaaaaatcta agtgtttcat aaatttgaga gtctgtgacc |
| 2341 | cacttacctt gcatctcaca ggtagacagt atataactaa caaccaaaga ctacatattg |
| 2401 | tcactgacac acacgttata atcatttatc atatatatac atacatgcat acactctcaa |
| 2461 | agcaaataat ttttcacttc aaaacagtat tgacttgtat accttgtaat ttgaaatatt |
| 2521 | ttctttgtta aaatagaatg gtatcaataa atagaccatt aatcagaaaa cagatcttga |
| 2581 | ttttttttct cttgaatgta cccttcaact gttgaatgtt taatagtaaa tcttatatgt |
| 2641 | ccttatttac tttttagctt tctctcaaat aaagtgtaac actagttgag ataacacatg |
| 2701 | aaagctcttt aaagggtcga tcgggaacag gaaaaaaaac ctatggaaaa tatgacaaca |
| 2761 | c |
| SEQ ID NO: 8 is an exemplary amino acid sequence of human CXCL9 (GENBANK ™ | |
| Accession No. NP_002407.1). | |
| 1 | MKKSGVLFLL GIILLVLIGV QGTPVVRKGR CSCISTNQGT IHLQSLKDLK QFAPSPSCEK |
| 61 | IEIIATLKNG VQTCLNPDSA DVKELIKKWE KQVSQKKKQK NGKKHQKKKV LKVRKSQRSR |
| 121 | QKKTT |
| SEQ ID NO: 9 is a nucleotide sequence encoding human CXCL10 (GENBANK ™ Accession | |
| No. NM_001565.4). | |
| 1 | gagacattcc tcaattgctt agacatattc tgagcctaca gcagaggaac ctccagtctc |
| 61 | agcaccatga atcaaactgc cattctgatt tgctgcctta tctttctgac tctaagtggc |
| 121 | attcaaggag tacctctctc tagaactgta cgctgtacct gcatcagcat tagtaatcaa |
| 181 | cctgttaatc caaggtcttt agaaaaactt gaaattattc ctgcaagcca attttgtcca |
| 241 | cgtgttgaga tcattgctac aatgaaaaag aagggtgaga agagatgtct gaatccagaa |
| 301 | tcgaaggcca tcaagaattt actgaaagca gttagcaagg aaaggtctaa aagatctcct |
| 361 | taaaaccaga ggggagcaaa atcgatgcag tgcttccaag gatggaccac acagaggctg |
| 421 | cctctcccat cacttcccta catggagtat atgtcaagcc ataattgttc ttagtttgca |
| 481 | gttacactaa aaggtgacca atgatggtca ccaaatcagc tgctactact cctgtaggaa |
| 541 | ggttaatgtt catcatccta agctattcag taataactct accctggcac tataatgtaa |
| 601 | gctctactga ggtgctatgt tcttagtgga tgttctgacc ctgcttcaaa tatttccctc |
| 661 | acctttccca tcttccaagg gtactaagga atctttctgc tttggggttt atcagaattc |
| 721 | tcagaatctc aaataactaa aaggtatgca atcaaatctg ctttttaaag aatgctcttt |
| 781 | acttcatgga cttccactgc catcctccca aggggcccaa attctttcag tggctaccta |
| 841 | catacaattc caaacacata caggaaggta gaaatatctg aaaatgtatg tgtaagtatt |
| 901 | cttatttaat gaaagactgt acaaagtaga agtcttagat gtatatattt cctatattgt |
| 961 | tttcagtgta catggaataa catgtaatta agtactatgt atcaatgagt aacaggaaaa |
| 1021 | ttttaaaaat acagatagat atatgctctg catgttacat aagataaatg tgctgaatgg |
| 1081 | ttttcaaaat aaaaatgagg tactctcctg gaaatattaa gaaagactat ctaaatgttg |
| 1141 | aaagatcaaa aggttaataa agtaattata actaa |
| SEQ ID NO: 10 is an exemplary amino acid sequence of human CXCL10 (GENBANK ™ | |
| Accession No. NP_001556.2). | |
| 1 | MNQTAILICC LIFLTLSGIQ GVPLSRTVRC TCISISNQPV NPRSLEKLEI IPASQFCPRV |
| 61 | EIIATMKKKG EKRCLNPESK AIKNLLKAVS KERSKRSP |
| SEQ ID NO: 11 is a nucleotide sequence encoding human CXCL11 (GENBANK ™ | |
| Accession No. NM_005409.5). | |
| 1 | gttcagcatt tctactcctt ccaagaagag cagcaaagct gaagtagcag cagcagcacc |
| 61 | agcagcaaca gcaaaaaaca aacatgagtg tgaagggcat ggctatagcc ttggctgtga |
| 121 | tattgtgtgc tacagttgtt caaggcttcc ccatgttcaa aagaggacgc tgtctttgca |
| 181 | taggccctgg ggtaaaagca gtgaaagtgg cagatattga gaaagcctcc ataatgtacc |
| 241 | caagtaacaa ctgtgacaaa atagaagtga ttattaccct gaaagaaaat aaaggacaac |
| 301 | gatgcctaaa tcccaaatcg aagcaagcaa ggcttataat caaaaaagtt gaaagaaaga |
| 361 | atttttaaaa atatcaaaac atatgaagtc ctggaaaaga gcatctgaaa aacctagaac |
| 421 | aagtttaact gtgactactg aaatgacaag aattctacag taggaaactg agacttttct |
| 481 | atggttttgt gactttcaac ttttgtacag ttatgtgaag gatgaaaggt gggtgaaagg |
| 541 | accaaaaaca gaaatacagt cttcctgaat gaatgacaat cagaattcca ctgcccaaag |
| 601 | gagtccaaca attaaatgga tttctaggaa aagctacctt aagaaaggct ggttaccatc |
| 661 | ggagtttaca aagtgctttc acgttcttac ttgttgcatt atacattcat gcatttctag |
| 721 | gctagagaac cttctagatt tgatgcttac aactattctg ttgtgactat gagaacattt |
| 781 | ctgtctctag aagtcatctg tctgtattga tctttatgct atattactat ctgtggttac |
| 841 | ggtggagaca ttgacattat tactggagtc aagcccttat aagtcaaaag catctatgtg |
| 901 | tcgtaaaaca ttcctcaaac attttttcat gcaaatacac acttctttcc ccaaacatca |
| 961 | tgtagcacat caatatgtag ggagacattc ttatgcatca tttggtttgt tttataacca |
| 1021 | attcattaaa tgtaattcat aaaatgtact atgaaaaaaa ttatacgcta tgggatactg |
| 1081 | gcaaaagtgc acatatttca taaccaaatt agtagcacca gtcttaattt gatgtttttc |
| 1141 | aacttttatt cattgagatg ttttgaagca attaggatat gtgtgtttac tgtacttttt |
| 1201 | gttttgatcc gtttgtataa atgatagcaa tatcttggac acatctgaaa tacaaaatgt |
| 1261 | ttttgtctac caaagaaaaa tgttgaaaaa taagcaaatg tatacctagc aatcactttt |
| 1321 | actttttgta attctgtctc ttagaaaaat acataatcta atcaatttct ttgttcatgc |
| 1381 | ctatatactg taaaatttag gtatactcaa gactagttta aagaatcaaa gtcatttttt |
| 1441 | tctctaataa actaccacaa cctttctttt ttaaaaaaa |
| SEQ ID NO: 12 is an exemplary amino acid sequence of human CXCL11 (GENBANK ™ | |
| Accession No. NP_005400.1). | |
| 1 | MSVKGMAIAL AVILCATVVQ GFPMFKRGRC LCIGPGVKAV KVADIEKASI MYPSNNCDKI |
| 61 | EVIITLKENK GQRCLNPKSK QARLIIKKVE RKNF |
| SEQ ID NO: 13 is a nucleotide sequence encoding human CXCL4 (GENBANK ™ Accession | |
| No. NM_002619.4). | |
| 1 | attggccaca gagacccagc ccgagtttcc catcgcactg agcactgaga tcctgctgga |
| 61 | agctctgccg cagcatgagc tccgcagccg ggttctgcgc ctcacgcccc gggctgctgt |
| 121 | tcctggggtt gctgctcctg ccacttgtgg tcgccttcgc cagcgctgaa gctgaagaag |
| 181 | atggggacct gcagtgcctg tgtgtgaaga ccacctccca ggtccgtccc aggcacatca |
| 241 | ccagcctgga ggtgatcaag gccggacccc actgccccac tgcccaactg atagccacgc |
| 301 | tgaagaatgg aaggaaaatt tgcttggacc tgcaagcccc gctgtacaag aaaataatta |
| 361 | agaaactttt ggagagttag ctactagctg cctacgtgtg tgcatttgct atatagcata |
| 421 | cttctttttt ccagtttcaa tctaactgtg aaagaacttc tgatatttgt gttatcctta |
| 481 | tgattttaaa taaacaaaat aaatcaagtt gtagtatagt caaaatactt cttaataata |
| 541 | gtgcaaaaat tgtgttgaca cataacaatt tcatggaaga aaaaaattcc ggtattttaa |
| 601 | gcaaaaagta ttttgaagga aggtgtgaat actggttatg cttggtgtta catgttggct |
| 661 | gatacatatt catgcattta catgattgca gtactttata gctacatatt taccttgacc |
| 721 | attattatta cctttgccaa taaatatcag taacacagat ggcttttaaa aaa |
| SEQ ID NO: 14 is an exemplary amino acid sequence of human CXCL4 (GENBANK ™ | |
| Accession No. NP_002610.1). | |
| 1 | MSSAAGFCAS RPGLLFLGLL LLPLVVAFAS AEAEEDGDLQ CLCVKTTSQV RPRHITSLEV |
| 61 | IKAGPHCPTA QLIATLKNGR KICLDLQAPL YKKIIKKLLE S |

### DETAILED DESCRIPTION

### I. Abbreviations

BM Bone Marrow
CO Coacervate
COI Collagen Orientation Index
Col-1 Collagen Type 1
Col-III Collagen Type III
Col-IV Collagen Type IV
CM Conditioned Media
CXCR3 Cysteine-X Amino Acid-Cysteine Receptor 3
DCN Decorin
DOPC 1,2-dioleoyl-sn-glycero-3-phosphocholine
DPPC 1,2-dipalmitoyl-sn-glycero-3-phosphocholine
ECM Extracellular Matrix
EGFR Epidermal Growth Factor Receptor
H&E Hematoxylin & Eosin
HB-EGF Heparin-Binding EGF-like growth factor
HMEC-1 Human Dermal Microvascular Endothelial Cells
H/ND Hypoxia and Nutrient Deprivation
HTS Hypertrophic Scars
IHMSC Immortalized Bone Marrow Mesenchymal Stem Cells
IL Interleukin
MMPs Matrix Metalloproteinases
MSC Mesenchymal Stem Cell
MT Masson's Trichrome
PBS Phosphate-buffered saline
PEAD Poly(Ethylene Arginyl Aspartate Diglyceride)
PI Propidium Iodide
PrhMSC Primary Human Bone-Marrow Mesenchymal Stem Cell
PSR Picrosirius Red
TGF-β₁ Transforming Growth Factor-Beta-1
TNC Tenascin-C
VEGF Vascular Endothelial Growth Factor

### II. Terms and Methods

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: The introduction of a composition (such as a controlled delivery composition disclosed herein) to a subject by a chosen route. Exemplary routes of administration for the agents and compositions disclosed herein include, but are not limited to, topical (including on a wound dressing, such as a single-use bandage), transdermal, oral, sublingual, injection (such as subcutaneous or intradermal injection), intranasal, inhalation and via a medical implant. As used herein, “administered concurrently” means administered at the same time (including in the same composition or in separate compositions) or during the same session.

Amphiregulin (AREG): A member of the epidermal growth factor family. The AREG protein is an autocrine growth factor as well as a mitogen for astrocytes, Schwann cells and fibroblasts. It is related to epidermal growth factor (EGF) and transforming growth factor alpha (TGF-alpha). The protein interacts with the EGF/TGF-alpha receptor to promote the growth of normal epithelial cells, and it inhibits the growth of certain aggressive carcinoma cell lines. AREG also functions in mammary gland, oocyte and bone tissue development. The AREG gene is associated with a psoriasis-like skin phenotype, and is also associated with other pathological disorders, including various types of cancers and inflammatory conditions. Nucleic acid and amino acid sequences for human AREG (and homologs thereof) are publicly available, such as under NCBI Gene ID 374.

Angiogenesis: The physiological process involving the growth of new blood vessels from pre-existing vessels. Angiogenesis is a normal and vital process in growth and development, as well as in wound healing.

C-X-C chemokine receptor 3 (CXCR3): A G protein-coupled receptor with selectivity for four chemokines, CXCL4/PF4 (platelet factor 4), CXCL9/Mig (monokine induced by interferon-γ), CXCL10/IP-10 (interferon-γ-inducible 10 kDa protein) and CXCL11/I-TAC (interferon-inducible T cell a-chemoattractant). Binding of chemokines to this protein induces cellular responses that are involved in leukocyte trafficking, most notably integrin activation, cytoskeletal changes and chemotactic migration. Alternatively spliced transcript variants encoding different isoforms have been found for this gene.

Chemokine (C-X-C motif) ligand 4 (CXCL4): A small cytokine belonging to the CXC chemokine family. CXCR4, also known as platelet factor 4 (PF4), is a 70-amino acid protein that is released from the alpha-granules of activated platelets and binds with high affinity to heparin. Its primary physiologic role is neutralization of heparin-like molecules on the endothelial surface of blood vessels, thereby inhibiting local antithrombin III activity and promoting coagulation. As a strong chemoattractant for neutrophils and fibroblasts, CXCL4 is believed to play a role in inflammation and wound repair. CXCL4 binds the B isoform of CXCR3 (CXCR3-B). Sequences for CXCL4 are publicly available (see, for example, GENBANK™ Gene ID 5196). Exemplary human CXCL4 nucleic acid and protein sequence are set forth herein as SEQ ID NOs: 13 and 14, respectively.

Chemokine (C-X-C motif) ligand 9 (CXCL9): A member of the CXC chemokine family. The CXCL9 protein is thought to be involved in T cell trafficking. CXCL9 binds to CXCR3 and is a chemoattractant for lymphocytes, but not for neutrophils. Nucleic acid and amino acid sequences for human CXCL9 (and homologs thereof) are publicly available, such as under NCBI Gene ID 4283. Exemplary human CXCL9 nucleic acid and protein sequence are set forth herein as SEQ ID NOs: 7 and 8, respectively.

Chemokine (C-X-C motif) ligand 10 (CXCL10): A chemokine of the CXC subfamily and a ligand for the receptor CXCR3. CXCL10 is also known as interferon-γ-inducible 10 kDa protein (IP-10). Binding of this protein to CXCR3 results in pleiotropic effects, including stimulation of monocytes, natural killer and T-cell migration, modulation of adhesion molecule expression, and inhibition of vessel formation. CXCL10 nucleic acid and amino acid sequences are publicly available, such as under NCBI Gene ID 3627 (see also GENBANK™ Accession No. P02778). Exemplary human CXCL10 nucleic acid and protein sequence are set forth herein as SEQ ID NOs: 9 and 10, respectively.

Chemokine (C-X-C motif) ligand 11 (CXCL11): A chemokine of the CXC subfamily and a ligand for the receptor CXCR3. The CXCL11 protein induces a chemotactic response in activated T-cells and is the dominant ligand for CXCR3. The CXCR11 gene contains 4 exons and at least three polyadenylation signals which may reflect cell-specific regulation of expression. IFN-γ is a potent inducer of transcription of this gene. Nucleic acid and amino acid sequences for CXCL11 are publicly available, such as under NCBI Gene ID 6373. Exemplary human CXCL11 nucleic acid and protein sequence are set forth herein as SEQ ID NOs: 11 and 12, respectively.

Cholesterol: A type of lipid molecule classified as a sterol. Cholesterol is an important structural component of biological membranes. The chemical formula for cholesterol is C₂₇H₁₆O and the IUPAC ID for cholesterol is (3β)-cholest-5-en-3-ol (see also PubChem CID 5997).

Coacervate: Spherical aggregates of colloidal droplets held together (and apart from their surrounding liquid environment) by hydrophobic forces. Coacervate droplets are generally about 0.1 to about 100 μm in diameter. Coacervates typically aggregate over time to form a bulk phase separation from the aqueous compartment. Coacervates can be used as controlled delivery vehicles for small molecules and protein therapeutics. Advantageous features of a coacervate for drug delivery include their high loading capacity and ability to self-assemble in aqueous media (see, e.g., Johnson and Wang, Expert Opin Drug Deliv 11 (12): 1829-1832, 2014). In some aspects herein, the coacervate includes a positively charged synthetic biodegradable poly(ethylene arginyl aspartate diglyceride) (PEAD) and a negatively charged heparin, which forms a 3-dimensional coacervate that envelopes around the protein cargo to be administered. Other coacervates can be used, such as those described in U.S. Patent Application Publication Nos. 2019/0117780 and 2021/0290770.

Collagen type I alpha 1 chain (COL1A1): A gene encoding the pro-alpha1 chains of type I collagen whose triple helix comprises two alpha1 chains and one alpha2 chain. Type I is a fibril-forming collagen found in most connective tissues and is abundant in bone, cornea, dermis and tendon. Mutations in the COL1A1 gene are associated with osteogenesis imperfecta types I-IV, Ehlers-Danlos syndrome type VIIA, Ehlers-Danlos syndrome Classical type, Caffey Disease and idiopathic osteoporosis. Reciprocal translocations between chromosomes 17 and 22, where this gene and the gene for platelet-derived growth factor beta are located, are associated with a particular type of skin tumor called dermatofibrosarcoma protuberans, resulting from unregulated expression of the growth factor. Two transcripts, resulting from the use of alternate polyadenylation signals, have been identified for this gene. Nucleic acid and amino acid sequences for human COL1A1 (and homologs thereof) are publicly available, such as under NCBI Gene ID 1227.

Collagen type I alpha 2 chain (COL1A2): A gene encoding the pro-alpha2 chain of type I collagen whose triple helix comprises two alpha1 chains and one alpha2 chain. Type I is a fibril-forming collagen found in most connective tissues and is abundant in bone, cornea, dermis and tendon. Mutations in this gene are associated with osteogenesis imperfecta types I-IV, Ehlers-Danlos syndrome type VIIB, recessive Ehlers-Danlos syndrome Classical type, idiopathic osteoporosis, and atypical Marfan syndrome. Symptoms associated with mutations in this gene, however, tend to be less severe than mutations in the gene for the alpha1 chain of type I collagen (COL1A1) reflecting the different role of alpha2 chains in matrix integrity. Three transcripts, resulting from the use of alternate polyadenylation signals, have been identified for this gene. Nucleic acid and amino acid sequences for human COL1A2 (and homologs thereof) are publicly available, such as under NCBI Gene ID 1278.

Collagen type III alpha chain (COL3A1): A gene encoding the pro-alpha1 chains of type III collagen, a fibrillar collagen that is found in extensible connective tissues such as skin, lung, uterus, intestine and the vascular system, frequently in association with type I collagen. Mutations in this gene are associated with Ehlers-Danlos syndrome types IV, and with aortic and arterial aneurysms. Two transcripts, resulting from the use of alternate polyadenylation signals, have been identified for this gene. Nucleic acid and amino acid sequences for human COL3A1 (and homologs thereof) are publicly available, such as under NCBI Gene ID 1281.

Conservative variants: “Conservative” amino acid substitutions are those substitutions that do not substantially affect or decrease an activity or antigenicity of a protein or peptide. For example, a protein disclosed herein can include at most about 1, at most about 2, at most about 3, at most about 4 or at most about 5 conservative substitutions (such as 1, 2, 3, 4, or 5 conservative substitutions, and retain biological activity, such as the ability to promote wound healing. Specific, non-limiting examples of a conservative substitution include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variant also includes the use of a substituted amino acid in place of an unsubstituted parent amino acid. Non-conservative substitutions are those that reduce an activity or antigenicity.

Controlled delivery composition: A composition in which the rate of release of the component(s) (e.g., drugs, therapeutic agents) is regulated, for example to achieve rapid release and/or delayed release of the component(s). In the context of the present disclosure, rapid release of the first agent that promotes wound closure occurs prior to (or more rapidly than) delayed (or less rapid) release of the second agent that inhibits scarring.

Decorin (DCN): A member of the small leucine-rich proteoglycan family of proteins. Alternative splicing results in multiple transcript variants, at least one of which encodes a preproprotein that is proteolytically processed to generate the mature protein. The DCN protein plays a role in collagen fibril assembly. Binding of this protein to multiple cell surface receptors mediates its role in tumor suppression, including a stimulatory effect on autophagy and inflammation and an inhibitory effect on angiogenesis and tumorigenesis. Mutations in the DCN gene are associated with congenital stromal corneal dystrophy in human patients. Nucleic acid and amino acid sequences for human DCN are publicly available, such as under NCBI Gene ID 1634. Exemplary human DCN nucleic acid and protein sequence are set forth herein as SEQ ID NOs: 5 and 6, respectively.

Epidermal growth factor (EGF): A member of the epidermal growth factor superfamily. The EGF preproprotein is proteolytically processed to generate the 53-amino acid epidermal growth factor peptide. This protein acts a potent mitogenic factor that plays an important role in the growth, proliferation and differentiation of numerous cell types. The EGF protein acts by binding with high affinity to the cell surface receptor, EGFR. Defects in this gene are the cause of hypomagnesemia type 4. Dysregulation of the EGF gene has been associated with the growth and progression of certain cancers. Alternative splicing results in multiple transcript variants, at least one of which encodes a preproprotein that is proteolytically processed. Nucleic acid and amino acid sequences for human EGF (and homologs thereof) are publicly available, such as under NCBI Gene ID 1950.

Fibrosis: A condition associated with the thickening and scarring of connective tissue. Often, fibrosis occurs in response to an injury, such as from a disease or condition that damages tissue. Fibrosis is an exaggerated wound healing response that when severe, can interfere with normal organ function. Fibrosis can occur in almost any tissue of the body, including in the lung (pulmonary fibrosis, cystic fibrosis, radiation-induced lung injury), liver (cirrhosis, biliary atresia), heart (arterial fibrosis, endomyocardial fibrosis, prior myocardial infarction), brain, skin (scleroderma, sclerosis), kidney, joints and intestine (Crohn's disease).

Growth factor protein: A type of protein that is capable of stimulating cell proliferation, cell survival, wound healing and/or cellular differentiation. Examples of growth factor proteins include, but are not limited to, vascular endothelial growth factor (VEGF), a member of the fibroblast growth factor (FGF) family (e.g., FGF1, FGF2, FGF3, FGF4, FGF5, FGF6, FGF7, FGF8, FGF9, FGF10, FGF16, FGF17, FGF18, FGF19, FGF20, FGF21, FGF22, FGF23), insulin growth factor (IGF), epidermal growth factor (EGF), heparin binding EGF-like growth factor (HB-EGF), transforming growth factor (TGF)-α, TGF-β, amphiregulin, nerve growth factor (NGF), brain-derived neurotrophic factor (BDNF), and platelet-derived growth factor (PDGF). In some aspects herein, the growth factor is EGF, TGF-α, amphiregulin, VEGF, FGF, or PDGF.

Heparin binding EGF like growth factor (HB-EGF): A growth factor with both growth factor and heparin binding activity. HB-EGF is involved in several processes, including epidermal growth factor receptor signaling pathway, positive regulation of protein kinase B signaling, and positive regulation of wound healing. HB-EGF is also implicated in glomerulosclerosis and perinatal necrotizing enterocolitis. Nucleic acid and amino acid sequences for human HB-EGF (and homologs thereof) are publicly available, such as under NCBI Gene ID 1839. Exemplary human HB-EGF nucleic acid and protein sequence are set forth herein as SEQ ID NOs: 3 and 4, respectively.

Hydrogel: A macromolecular polymer gel comprised of a network of crosslinked polymer chains.

Interleukin-1 beta (IL-1β): A cytokine produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. Nucleic acid and amino acid sequences for human IL-1β (and homologs thereof) are publicly available, such as under NCBI Gene ID 3553.

Interleukin-2 (IL-2): A secreted cytokine produced by activated CD4+ and CD8+ T lymphocytes important for the proliferation of T and B lymphocytes. The IL-2 receptor is a heterotrimeric protein complex whose gamma chain is also shared by IL-4 and IL-7. Nucleic acid and amino acid sequences for human IL-2 (and homologs thereof) are publicly available, such as under NCBI Gene ID 3558.

Interleukin-4 (IL-4): A pleiotropic cytokine produced by activated T cells. The IL-4 receptor also binds to IL-13, which may contribute to the many overlapping functions of IL-4 and IL-13. IL-4 plays an important role tissue repair, promotes allergic airway inflammation, and regulates a variety of human host responses such as allergic, anti-parasitic, wound healing, and acute inflammation. This cytokine has also been reported to promote resolution of neutrophil-mediated acute lung injury. Two alternatively spliced transcript variants of the IL-4 gene encoding distinct isoforms have been reported. Nucleic acid and amino acid sequences for human IL-4 (and homologs thereof) are publicly available, such as under NCBI Gene ID 3565.

Interleukin-10 (IL-10): A cytokine produced primarily by monocytes and to a lesser extent by lymphocytes. IL-10 has pleiotropic effects in immunoregulation and inflammation. It down-regulates the expression of Th1 cytokines, MHC class II antigens, and costimulatory molecules on macrophages. It also enhances B cell survival, proliferation, and antibody production. In addition, IL-10 can block NF-κB activity and is involved in the regulation of the JAK-STAT signaling pathway. Nucleic acid and amino acid sequences for human IL-10 (and homologs thereof) are publicly available, such as under NCBI Gene ID 3586.

Laminin subunit beta 1 (LAMB1): An extracellular matrix glycoprotein that is a major noncollagenous constituent of basement membranes. Laminins have been implicated in a wide variety of biological processes including cell adhesion, differentiation, migration, signaling, neurite outgrowth and metastasis. Laminins are composed of 3 non identical chains: laminin alpha, beta and gamma (formerly A, B1, and B2, respectively) and they form a cruciform structure consisting of 3 short arms, each formed by a different chain, and a long arm composed of all 3 chains. Each laminin chain is a multidomain protein encoded by a distinct gene. The beta 1 chain has 7 structurally distinct domains which it shares with other beta chain isomers. The C-terminal helical region containing domains I and II are separated by domain alpha, domains III and V contain several EGF-like repeats, and domains IV and VI have a globular conformation. LAMB1 is expressed in most tissues that produce basement membranes. Nucleic acid and amino acid sequences for human LAMB1 (and homologs thereof) are publicly available, such as under NCBI Gene ID 3912.

Lipid: An inclusive term for fats and fat-derived materials. Lipids include esters of fatty acids (simple lipids, such as fats, sterols, waxes, and triglycerides) or closely related substances (compound lipids, such as phospholipids). In the context of the present disclosure, the lipids include cholesterol as well as saturated or unsaturated lipids. A “saturated lipid” refers to a lipid in which the fatty acid chains all have single bonds. An “unsaturated lipid” refers to a lipid containing a high proportion of fatty acid molecules with at least one double bond. Non-limiting examples of lipids that can be used in the disclosed controlled delivery compositions include cholesterol and any one or more the following:

| Lipid | Chemical Name | Type |
|---|---|---|
| 18:1 (Δ9-Cis) | 1,2-dioleoyl-sn-glycero-3-phosphocholine | Unsaturated |
| PC (DOPC) | | |
| 18:1 (Δ9-Cis) | 1,2-dioleoyl-sn-glycero-3-phospho-(1′-rac- | Unsaturated |
| PG (DOPG) | glycerol) | |
| 18:1 (Δ9-Cis) | 1,2-dioleoyl-sn-glycero-3- | Unsaturated |
| PE (DOPE) | phosphoethanolamine | |
| 16:0-18:1 PE | 1-palmitoyl-2-oleoyl-sn-glycero-3- | Unsaturated |
| (POPE) | phosphoethanolamine | |
| 16:0-18:1 PC | 1-palmitoyl-2-oleoyl-sn-glycero-3- | Unsaturated |
| (POPC) | phosphocholine | |
| 16:0-18:1 PS | 1-palmitoyl-2-oleoyl-sn-glycero-3- | Unsaturated |
| (POPS) | phospho-L-serine | |
| 18:1 DGS- | 1,2-dioleoyl-sn-glycero-3-[(N-(5-amino-1- | Unsaturated |
| NTA(Ni) | carboxypentyl)iminodiacetic | |
| | acid)succinyl] | |
| 16:0 PC | 1,2-dipalmitoyl-sn-glycero-3- | Saturated |
| (DPPC) | phosphocholine | |
| 18:0 PC | 1,2-distearoyl-sn-glycero-3- | Saturated |
| (DSPC) | phosphocholine | |
| 16:0 PS | 1,2-dipalmitoyl-sn-glycero-3-phospho-L- | Saturated |
| (DPPS) | serine | |
| 18:0 PS | 1,2-distearoyl-sn-glycero-3-phospho-L- | Saturated |
| (DSPS) | serine | |
| 16:0 PE | 1,2-dipalmitoyl-sn-glycero-3- | Saturated |
| (DPPE) | phosphoethanolamine | |
| 18:0 PE | 1,2-distearoyl-sn-glycero-3- | Saturated |
| (DSPE) | phosphoethanolamine | |
| 16:0 PG | 1,2-dipalmitoyl-sn-glycero-3-phospho-(1′- | Saturated |
| (DPPG) | rac-glycerol) | |
| 18:0 PG | 1,2-distcaroyl-sn-glyccro-3-phospho-(1′- | Saturated |
| (DSPG) | rac-glycerol) | |
| Cholesterol | (3β)-cholest-5-en-3-ol | Sterol |

Lipids for use in the disclosed methods and compositions are described in, for example, Deshpande et al., Nat Commun 7:10447, 2016; Deshpande et al., Biomicrofluidics 11:034106, 2017; Deshpande et al., Nat Commun 10:1800, 2019; Last et al., ACS Nano 14:4487-4498, 2020; Cakmak et al., Langmuir 35 (24): 7830-7840, 2019; Deshpande and Dekker, Nature Protocols 13 (5): 856-874, 2018; Cakmak et al., Langmuir 37:10366-10375, 2021; and Zhang et al., J Am Chem Soc 143:2866-2874, 2021. The lipids listed in the table above are commercially available from a variety of sources, for example, Avanti Polar Lipids (Birmingham, AL), Sigma-Aldrich (St. Louis, MO), Echelon Biosciences (Salt Lake City, UT), and Anatrace Lipids (Maumee, OH).

Matricellular proteins: A class of non-structural, secreted proteins found in the extracellular matrix. Matricellular proteins have a variety of different functions mediated by interacting with cell-surface receptors, proteases, hormones, and structural matrix proteins (e.g., collagen). Matricellular proteins are further characterized by high levels of expression during development, tissue remodeling and response to injury, as well as induction of de-adhesion (see, e.g., Bornstein, J Cell Commun Signal 3 (3-4): 163-165, 2009). Examples of matricellular proteins include, but are not limited to, secreted protein acidic and cysteine rich (SPARC), thrombospondin (e.g., TSP-1), laminin B1, collagen III and tenascin-C.

Mucosa: Moist tissue that covers the inside surface of some parts of the body, such as the nose, mouth, anus/rectum, vagina, lungs, urinary tract and digestive tract. Glands in the mucosa produce mucous.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the proteins and compositions disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Platelet derived growth factor subunit alpha (PDGFA): A member of the protein family comprised of both platelet-derived growth factors (PDGF) and vascular endothelial growth factors (VEGF). The PDGFA preproprotein is proteolytically processed to generate platelet-derived growth factor subunit A, which can homodimerize, or alternatively, heterodimerize with the related platelet-derived growth factor subunit B. These proteins bind and activate PDGF receptor tyrosine kinases, which play a role in a wide range of developmental processes. Nucleic acid and amino acid sequences for human PDGFA (and homologs thereof) are publicly available, such as under NCBI Gene ID 5154.

Platelet derived growth factor subunit beta (PDGFB): A member of the protein family comprised of both platelet-derived growth factors (PDGF) and vascular endothelial growth factors (VEGF). The PDGFB preproprotein is proteolytically processed to generate platelet-derived growth factor subunit B, which can homodimerize, or alternatively, heterodimerize with the related platelet-derived growth factor subunit A. These proteins bind and activate PDGF receptor tyrosine kinases, which play a role in a wide range of developmental processes. Nucleic acid and amino acid sequences for human PDGFA (and homologs thereof) are publicly available, such as under NCBI Gene ID 5155.

Poly(lactic-co-glycolic acid) (PLGA): A biodegradable and biocompatible co-polymer of glycolic acid and lactic acid.

Scarring: The formation of fibrous tissue in response to a wound, injury or disease.

Secreted protein acidic and cysteine rich (SPARC): A gene encoding the pro-alpha2 chain of type I collagen whose triple helix comprises two alpha1 chains and one alpha2 chain. Type I is a fibril-forming collagen found in most connective tissues and is abundant in bone, cornea, dermis and tendon. Mutations in SPARC are associated with osteogenesis imperfecta types I-IV, Ehlers-Danlos syndrome type VIIB, recessive Ehlers-Danlos syndrome Classical type, idiopathic osteoporosis, and atypical Marfan syndrome. Three transcripts, resulting from the use of alternate polyadenylation signals, have been identified for this gene. Nucleic acid and amino acid sequences for human SPARC (and homologs thereof) are publicly available, such as under NCBI Gene ID 6678.

Sequence identity: The similarity between nucleic acid or amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a particular polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. In addition, Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 90%, 95%, 96%, 97%, 98% or 99% sequence identity counted over the full length alignment with the amino acid sequence of the polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment can be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals.

Tenascin-C (TNC): An extracellular matrix protein with a spatially and temporally restricted tissue distribution. The TNC protein is homohexameric with disulfide-linked subunits and contains multiple EGF-like and fibronectin type-III domains. It is implicated in guidance of migrating neurons as well as axons during development, synaptic plasticity, and neuronal regeneration. Nucleic acid and amino acid sequences for human TNC (and homologs thereof) are publicly available, such as under NCBI Gene ID 3371. Exemplary human TNC nucleic acid and protein sequence are set forth herein as SEQ ID NOs: 1 and 2, respectively (see also UniProt P24821). In some aspects, the TNC protein is a biologically active fragment of TNC that includes the EGF-like repeats. In some aspects, the TNC protein is a biologically active fragment of TNC that includes the integrin-binding domains. In other aspects, the TNC protein is full-length TNC.

Therapeutically effective amount: A quantity of a specified agent, such as an agent that promotes wound closure (e.g., HB-EGF, TNC, a matricellular protein, or a growth factor) or an agent that inhibits scarring (e.g., DCN, a CXCR3 ligand, collagen type I, or IL-10), sufficient to achieve a desired effect in a subject being treated with that agent. In some examples, the therapeutically effective amount is an amount necessary to increase wound closure by at least 10%, at least 20%, at least 30%, at least 40%, at least 50%, at least 60%, at least 70%, at least 80%, at least 90%, or at least 100% or more, relative to wound closure in the absence of treatment. In other examples, the therapeutically effective amount is an amount necessary to inhibit scarring by at least 10%, at least 20%, at least 30%, at least 40%, at least 50%, at least 60%, at least 70%, at least 80%, at least 90%, or at least 100% or more, relative to scarring in the absence of treatment.

Thrombospondin 1 (THBS1): A subunit of a disulfide-linked homotrimeric protein. The THBS1 protein is an adhesive glycoprotein that mediates cell-to-cell and cell-to-matrix interactions. This protein can bind to fibrinogen, fibronectin, laminin, type V collagen and integrins alpha-V/beta-1. The THSB1 protein has been shown to play roles in platelet aggregation, angiogenesis, and tumorigenesis. Nucleic acid and amino acid sequences for human THBS1 (and homologs thereof) are publicly available, such as under NCBI Gene ID 7057.

Thrombospondin 2 (THBS2): A member of the thrombospondin family. The THBS2 protein is a disulfide-linked homotrimeric glycoprotein that mediates cell-to-cell and cell-to-matrix interactions. This protein has been shown to function as a potent inhibitor of tumor growth and angiogenesis. Studies of the mouse counterpart suggest that this protein may modulate the cell surface properties of mesenchymal cells and be involved in cell adhesion and migration. Nucleic acid and amino acid sequences for human THBS2 (and homologs thereof) are publicly available, such as under NCBI Gene ID 7058.

Transforming growth factor alpha (TGFA): A growth factor that is a ligand for the epidermal growth factor receptor, which activates a signaling pathway for cell proliferation, differentiation and development. The TGF-α protein may act as either a transmembrane-bound ligand or a soluble ligand. The TGFA gene has been associated with many types of cancers, and it may also be involved in some cases of cleft lip/palate. Alternatively spliced transcript variants encoding different isoforms have been found for this gene. Nucleic acid and amino acid sequences for human TGFA (and homologs thereof) are publicly available, such as under NCBI Gene ID 7039.

Vascular endothelial growth factor A (VEGFA): A member of the PDGF/VEGF growth factor family. VEGFA is a heparin-binding protein that exists as a disulfide-linked homodimer. This growth factor induces proliferation and migration of vascular endothelial cells and is important for both physiological and pathological angiogenesis. Nucleic acid and amino acid sequences for human VEGFA (and homologs thereof) are publicly available, such as under NCBI Gene ID 7422.

Vascular endothelial growth factor B (VEGFB): A member of the PDGF/VEGF family. VEGF family members regulate the formation of blood vessels and are involved in endothelial cell physiology. VEGFB is a ligand for VEGFR-1 (vascular endothelial growth factor receptor 1) and NRP-1 (neuropilin-1). Nucleic acid and amino acid sequences for human VEGFB (and homologs thereof) are publicly available, such as under NCBI Gene ID 7423.

Vascular endothelial growth factor C (VEGFC): A member of the PDGF/VEGF family. VEGFC promotes angiogenesis and endothelial cell growth, and can also affect the permeability of blood vessels. The VEGFC proprotein is further cleaved into a fully processed form that can bind and activate VEGFR-2 and VEGFR-3 receptors. Nucleic acid and amino acid sequences for human VEGFC (and homologs thereof) are publicly available, such as under NCBI Gene ID 7424.

Wound: Any type of damage or breakage on the surface of the skin or mucosa. In some aspects herein, the would is a dermal wound, such as a laceration, a puncture wound, an abrasion, a surgical wound, a burn, an ulcer or a pressure sore. In other aspects, the wound is a mucosal wound, such as a wound in the nose, mouth, anus/rectum, vagina or lung.

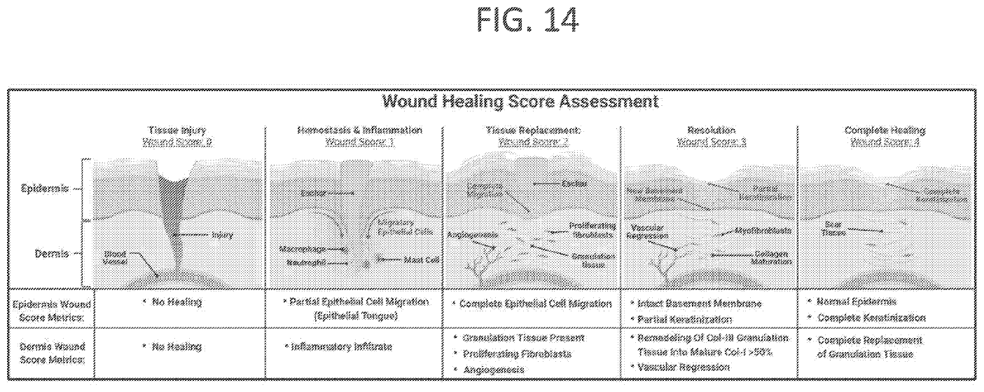

Wound closure: The process of how an excisional wound (loss of epidermal and/or dermal tissue) heals over time. A schematic of wound closure is shown in FIG. 14.

### III. Overview of Several Aspects

The present disclosure describes controlled delivery methods and compositions that enhance wound healing (for example, improve, enhance or accelerate wound closure) and reduce fibrotic scarring. Specifically provided are methods of treating a wound, such as a dermal wound or a mucosal wound, in a subject by administering to the subject a therapeutically effective amount of a first agent that promotes wound closure and a therapeutically effective amount of a second agent that inhibits scarring. In the disclosed methods, the first agent is administered prior to the second agent (such as at least one day to three weeks prior to the second agent), or the two agents are administered concurrently in a controlled delivery composition that allows rapid release of the first agent and delayed release of the second agent. Controlled delivery compositions that permit rapid release of the first agent and delayed release of the second agent are also described.

### A. Methods for Treating a Wound

Provided herein are methods of treating a wound in the subject by administering to the subject a therapeutically effective amount of a first agent that promotes wound closure and a therapeutically effective amount of a second agent that inhibits scarring.

In some aspects, the first agent includes heparin binding EGF-like growth factor (HB-EGF), tenascin-C (TNC), a growth factor, a matricellular protein, interleukin (IL)-1, IL-2 or IL-4. Growth factors include, for example, epidermal growth factor (EGF), vascular endothelial growth factor (VEGF), members of the fibroblast growth factor (FGF) family, transforming growth factor (TGF)-α, amphiregulin, and platelet-derived growth factor (PDGF). Matricellular proteins include, for example, secreted protein acidic and cysteine rich (SPARC), thrombospondin, laminin B1, and collagen type III. In some aspects, the first agent is a biologically active fragment of HB-EGF, TNC, a growth factor (such as EGF, VEGF, PDGF, TGF-α, amphiregulin, or PDGF), a matricellular protein (such as SPARC, thrombospondin, laminin B1 or collagen type III), IL-1, IL-2 or IL-4, wherein the fragment retains the biological activity of the respective full-length protein. In some examples, the biologically active fragment includes one or more EGF-like repeats or one or more integrin binding domains.

In some examples, the first agent is TNC. In particular examples, the amino acid sequence of TNC is at least 85%, at least 90%, at least 95%, at least 96%, at least 97%, at least 98%, at least 99% or 100% identical to SEQ ID NO: 2. In specific non-limiting examples, the amino acid sequence of TNC consists of SEQ ID NO: 2.

In some examples, the first agent is HB-EGF. In particular examples, the amino acid sequence of HB-EGF is at least 85%, at least 90%, at least 95%, at least 96%, at least 97%, at least 98%, at least 99% or 100% identical to SEQ ID NO: 4. In specific non-limiting examples, the amino acid sequence of HB-EGF consists of SEQ ID NO: 4.

In some aspects, the second agent includes decorin (DCN), a CXCR3 ligand, collagen type I, or IL-10. CXCR3 ligands include, for example, CXCL4, CXCL9, CXCL10 and CXCL11. In some aspects, the second agent is a biologically active fragment of DCN, a CXCR3 ligand (such as CXCL4, CXCL9, CXCL10 or CXCL11), collagen type I or IL-10 that retains the biological activity of the respective full-length protein.

In some examples, the second agent is DCN. In particular examples, the amino acid sequence of DCN is at least 85%, at least 90%, at least 95%, at least 96%, at least 97%, at least 98%, at least 99% or 100% identical to SEQ ID NO: 6. In specific non-limiting examples, the amino acid sequence of DNC consists of SEQ ID NO: 6.

In some examples, the second agent is CXCL9. In particular examples, the amino acid sequence of CXCL9 is at least 85%, at least 90%, at least 95%, at least 96%, at least 97%, at least 98%, at least 99% or 100% identical to SEQ ID NO: 8. In specific non-limiting examples, the amino acid sequence of CXCL9 consists of SEQ ID NO: 8.

In some examples, the second agent is CXCL10. In particular examples, the amino acid sequence of CXCL10 is at least 85%, at least 90%, at least 95%, at least 96%, at least 97%, at least 98%, at least 99% or 100% identical to SEQ ID NO: 10. In specific non-limiting examples, the amino acid sequence of CXCL10 consists of SEQ ID NO: 10.

In some examples, the second agent is CXCL11. In particular examples, the amino acid sequence of CXCL11 is at least 85%, at least 90%, at least 95%, at least 96%, at least 97%, at least 98%, at least 99% or 100% identical to SEQ ID NO: 12. In specific non-limiting examples, the amino acid sequence of CXCL11 consists of SEQ ID NO: 12.

In some examples, the second agent is CXCL4. In particular examples, the amino acid sequence of CXCL4 is at least 85%, at least 90%, at least 95%, at least 96%, at least 97%, at least 98%, at least 99% or 100% identical to SEQ ID NO: 14. In specific non-limiting examples, the amino acid sequence of CXCL4 consists of SEQ ID NO: 14.

In some aspects of the disclosed methods, the first agent is administered to the subject prior to administration of the second agent. In some examples, the first agent is administered at least one day prior to administration of the second agent, such as at least 2 days, at least 3 days, at least 4 days, at least 5 days, at least 6 days, at least 1 week, at least 2 weeks or at least 3 weeks prior to administration of the second agent. In some examples, the first agent and/or second agent are encapsulated in a coacervate, hydrogel and/or liposome. In specific examples, the coacervate includes poly(ethylene arginyl aspartate diglyceride) (PEAD) and heparin.

In other aspects of the disclosed methods, the first agent and the second agent are administered concurrently in a controlled delivery composition that permits rapid release of the first agent and delayed release of the second agent.

In some examples, the controlled delivery composition includes a hydrogel, and the hydrogel includes the first agent and the second agent encapsulated in a coacervate, such that upon administration of the controlled delivery composition, the first agent is released from the hydrogel prior to release of the second agent from the coacervate. In specific examples, the coacervate includes poly(ethylene arginyl aspartate diglyceride) (PEAD) and heparin.

In other examples, the controlled delivery composition includes a first coacervate that includes the first agent, and a second coacervate that includes the second agent, wherein the second coacervate is encapsulated by lipids, thereby forming a lipo-coacervate, such that upon administration of the controlled delivery composition, the first agent is released from the first coacervate prior to release of the second agent from the lipo-coacervate. In particular examples, the lipids of the lipo-coacervate include cholesterol and at least one unsaturated lipid and/or at least one saturated lipid. The at least one unsaturated lipid can be, for example, 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and/or 18:1 DGS-NTA (Ni). The at least one saturated lipid can be, for example, 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and/or 18:0 PG (DSPG). In one non-limiting example, the lipids of the lipo-coacervate include cholesterol, DOPC and DSPG. In another non-limiting example, the lipids of the lipo-coacervate include cholesterol, DPPC and DSPG.

In yet other examples, the controlled delivery composition includes a first coacervate that includes the first agent, wherein the first coacervate is encapsulated by lipids, thereby forming a first lipo-coacervate; and a second coacervate that includes the second agent, wherein the second coacervate is encapsulated by lipids, thereby forming a second lipo-coacervate. Upon administration of the controlled delivery composition, the first agent is released from the first lipo-coacervate prior to release of the second agent from the second lipo-coacervate.

In particular examples, the first lipo-coacervate includes cholesterol and at least one unsaturated lipid selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni). In several examples, the first lipo-coacervate further includes a saturated lipid, such as, but not limited to 18:0 PG (DSPG). In one non-limiting example, the first lipo-coacervate includes cholesterol, DOPC and DSPG.

In particular examples, the second lipo-coacervate includes cholesterol and at least one saturated lipid selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG). In specific non-limiting examples, the second lipo-coacervate includes cholesterol, DPPC and DSPG.

In some examples, the first coacervate and/or the second coacervate include poly(ethylene arginyl aspartate diglyceride) (PEAD) and/or heparin.

In some aspects of the disclosed methods, the wound is a dermal wound. In specific examples, the dermal wound includes a laceration, a puncture wound, an abrasion, a surgical wound, a burn, an ulcer or a pressure sore.

In other aspects of the disclosed methods, the wound is a mucosal wound. In specific examples, the mucosal wound is in the nose, mouth, rectum, anus, vagina or lung.

In some aspects of the disclosed methods, the route of administration is topical. For example, topical administration can include application of the agent or composition at or adjacent to the site of the wound, such as by spraying (spray or aerosol), swabbing, wiping, on a wound dressing (such as a single-use bandage) or any other means for applying the composition to the wound. In other aspects, administration includes injection at or near the site of the wound.

### B. Controlled Delivery Compositions

Also provided herein are controlled delivery compositions for treating a wound, such as a dermal or mucosal wound. The controlled delivery compositions include a therapeutically effective amount of a first agent that promotes wound closure and a therapeutically effective amount of a second agent that inhibits scarring, such that the controlled delivery compositions permit rapid release of the first agent and delayed release of the second agent.

In some aspects, the first agent includes heparin binding EGF-like growth factor (HB-EGF), tenascin-C (TNC), a growth factor, a matricellular protein, interleukin (IL)-1, IL-2 or IL-4. Growth factors include, for example, epidermal growth factor (EGF), vascular endothelial growth factor (VEGF), members of the fibroblast growth factor (FGF) family, transforming growth factor (TGF)-α, amphiregulin, and platelet-derived growth factor (PDGF). Matricellular proteins include, for example, secreted protein acidic and cysteine rich (SPARC), thrombospondin, laminin B1, and collagen type III.

In some examples, the first agent is TNC. In particular examples, the amino acid sequence of TNC is at least 85%, at least 90%, at least 95%, at least 96%, at least 97%, at least 98%, at least 99% or 100% identical to SEQ ID NO: 2. In specific non-limiting examples, the amino acid sequence of TNC consists of SEQ ID NO: 2.

In some examples, the first agent is HB-EGF. In particular examples, the amino acid sequence of HB-EGF is at least 85%, at least 90%, at least 95%, at least 96%, at least 97%, at least 98%, at least 99% or 100% identical to SEQ ID NO: 4. In specific non-limiting examples, the amino acid sequence of HB-EGF consists of SEQ ID NO: 4.

In some aspects, the second agent includes decorin (DCN), a CXCR3 ligand, collagen type I, or IL-10. CXCR3 ligands include, for example, CXCL4, CXCL9, CXCL10 and CXCL11.

In some examples, the second agent is DCN. In particular examples, the amino acid sequence of DCN is at least 85%, at least 90%, at least 95%, at least 96%, at least 97%, at least 98%, at least 99% or 100% identical to SEQ ID NO: 6. In specific non-limiting examples, the amino acid sequence of DNC consists of SEQ ID NO: 6.

In some examples, the second agent is CXCL9. In particular examples, the amino acid sequence of CXCL9 is at least 85%, at least 90%, at least 95%, at least 96%, at least 97%, at least 98%, at least 99% or 100% identical to SEQ ID NO: 8. In specific non-limiting examples, the amino acid sequence of CXCL9 consists of SEQ ID NO: 8.

In some examples, the second agent is CXCL10. In particular examples, the amino acid sequence of CXCL10 is at least 85%, at least 90%, at least 95%, at least 96%, at least 97%, at least 98%, at least 99% or 100% identical to SEQ ID NO: 10. In specific non-limiting examples, the amino acid sequence of CXCL10 consists of SEQ ID NO: 10.

In some examples, the second agent is CXCL11. In particular examples, the amino acid sequence of CXCL11 is at least 85%, at least 90%, at least 95%, at least 96%, at least 97%, at least 98%, at least 99% or 100% identical to SEQ ID NO: 12. In specific non-limiting examples, the amino acid sequence of CXCL11 consists of SEQ ID NO: 12.

In some examples, the second agent is CXCL4. In particular examples, the amino acid sequence of CXCL4 is at least 85%, at least 90%, at least 95%, at least 96%, at least 97%, at least 98%, at least 99% or 100% identical to SEQ ID NO: 14. In specific non-limiting examples, the amino acid sequence of CXCL4 consists of SEQ ID NO: 14.

In some aspects, the controlled delivery composition includes a hydrogel, and the hydrogel includes the first agent and the second agent encapsulated in a coacervate, such that upon administration of the controlled delivery composition, the first agent is released from the hydrogel prior to release of the second agent from the coacervate. In specific examples, the coacervate includes poly(ethylene arginyl aspartate diglyceride) (PEAD) and heparin.

In other aspects, the controlled delivery composition includes a first coacervate that includes the first agent, and a second coacervate that includes the second agent, wherein the second coacervate is encapsulated by lipids, thereby forming a lipo-coacervate, such that upon administration of the controlled delivery composition, the first agent is released from the first coacervate prior to release of the second agent from the lipo-coacervate. In some examples, the lipids of the lipo-coacervate include cholesterol and at least one unsaturated lipid and/or at least one saturated lipid. In particular examples, the at least one unsaturated lipid is selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni). In particular examples, the at least one saturated lipid is selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG). In one non-limiting example, the lipids of the lipo-coacervate include cholesterol, DOPC and DSPG. In another non-limiting example, the lipids of the lipo-coacervate include cholesterol, DPPC and DSPG.

In yet other aspects, the controlled delivery composition includes a first coacervate that includes the first agent, wherein the first coacervate is encapsulated by lipids, thereby forming a first lipo-coacervate; and a second coacervate that includes the second agent, wherein the second coacervate is encapsulated by lipids, thereby forming a second lipo-coacervate. Upon administration of the controlled delivery composition, the first agent is released from the first lipo-coacervate prior to release of the second agent from the second lipo-coacervate.

In some examples, the first lipo-coacervate includes cholesterol and at least one unsaturated lipid selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni). In several examples, the first lipo-coacervate further includes a saturated lipid, such as, but not limited to 18:0 PG (DSPG). In one non-limiting example, the first lipo-coacervate includes cholesterol, DOPC and DSPG.

In some examples, the second lipo-coacervate includes cholesterol and at least one saturated lipid selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG). In specific non-limiting examples, the second lipo-coacervate includes cholesterol, DPPC and DSPG.

In some examples, the first coacervate and/or the second coacervate include poly(ethylene arginyl aspartate diglyceride) (PEAD) and/or heparin.

In some aspects of the disclosed controlled delivery composition, the wound to be treated is a dermal wound. In specific examples, the dermal wound includes a laceration, a puncture wound, an abrasion, a surgical wound, a burn, an ulcer or a pressure sore.

In other aspects of the disclosed controlled delivery compositions, the wound to be treated is a mucosal wound. In specific examples, the mucosal wound is in the nose, mouth, rectum, anus, vagina or lung.

In some aspects, the controlled delivery composition is formulated for topical administration, such as for spraying (spray or aerosol), swabbing, wiping, on a wound dressing (such as a single-use bandage) or any other means for applying the composition to the wound. In other aspects, the controlled delivery composition is formulated for administration by injection, such as injection at or near the site of the wound.

### IV. Exemplary Aspects

Aspect 1. A method of treating a wound in a subject, comprising administering to the subject a therapeutically effective amount of a first agent that promotes wound closure and a therapeutically effective amount of a second agent that inhibits scarring, wherein:
(i) the first agent is administered to the subject prior to administration of the second agent; or
(ii) the first agent and the second agent are administered concurrently in a controlled delivery composition that permits rapid release of the first agent and delayed release of the second agent.

Aspect 2. The method of aspect 1, wherein the first agent comprises heparin binding EGF-like growth factor (HB-EGF), tenascin-C (TNC), a growth factor, a matricellular protein or a biologically active fragment of a matricellular protein, interleukin (IL)-1, IL-2, or IL-4.

Aspect 3. The method of aspect 2, wherein the growth factor is selected from epidermal growth factor (EGF), vascular endothelial growth factor (VEGF), a member of the fibroblast growth factor (FGF) family, transforming growth factor (TGF)-α, amphiregulin, and platelet-derived growth factor (PDGF).

Aspect 4. The method of aspect 2, wherein the matricellular protein is selected from secreted protein acidic and cysteine rich (SPARC), thrombospondin, laminin B1, and collagen type III.

Aspect 5. The method of any one of aspects 1-4, wherein the second agent comprises decorin (DCN), a CXCR3 ligand, collagen type I, or IL-10.

Aspect 6. The method of clam 5, wherein the CXCR3 ligand is selected from CXCL4, CXCL9, CXCL10 and CXCL11, or is selected from a biologically active fragment of CXCL4, CXCL9, CXCL10 and CXCL11.

Aspect 7. The method of aspect 1 (i), wherein the first agent is administered at least one day prior to administration of the second agent.

Aspect 8. The method of aspect 7, wherein the first agent is administered at least 2 days, at least 3 days, at least 4 days, at least 5 days, at least 6 days, at least 1 week, at least 2 weeks or at least 3 weeks prior to administration of the second agent.

Aspect 9. The method of aspect 1 (ii), wherein the controlled delivery composition comprises a hydrogel and the hydrogel comprises:
the first agent; and
the second agent encapsulated in a coacervate,
wherein upon administration of the controlled delivery composition, the first agent is released from the hydrogel prior to release of the second agent from the coacervate.

Aspect 10. The method of aspect 1 (ii), wherein the controlled delivery composition comprises:
a first coacervate comprising the first agent;
a second coacervate comprising the second agent, wherein the second coacervate is encapsulated by lipids, thereby forming a lipo-coacervate,
wherein upon administration of the controlled delivery composition, the first agent is released from the first coacervate prior to release of the second agent from the lipo-coacervate.

Aspect 11. The method of aspect 10, wherein the lipids of the lipo-coacervate comprise:
cholesterol; and
at least one unsaturated lipid and/or at least one saturated lipid.

Aspect 12. The method of aspect 11, wherein:
the at least one unsaturated lipid is selected from 18:1 (Δ9-Cis) PC (DOPC), 18:1 (A9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni); and/or
the at least one saturated lipid is select from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

Aspect 13. The method of aspect 11 or aspect 12, wherein the lipids of the lipo-coacervate comprise cholesterol, DOPC and DSPG.

Aspect 14. The method of aspect 11 or aspect 12, wherein the lipids of the lipo-coacervate comprise cholesterol, DPPC and DSPG.

Aspect 15. The method of aspect 1 (ii), wherein the controlled delivery composition comprises:
a first coacervate comprising the first agent, wherein the first coacervate is encapsulated by lipids, thereby forming a first lipo-coacervate; and
a second coacervate comprising the second agent, wherein the second coacervate is encapsulated by lipids, thereby forming a second lipo-coacervate,
wherein upon administration of the controlled delivery composition, the first agent is released from the first lipo-coacervate prior to release of the second agent from the second lipo-coacervate.

Aspect 16. The method of aspect 15, wherein the first lipo-coacervate comprises cholesterol and at least one unsaturated lipid selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA(Ni).

Aspect 17. The method of aspect 16, wherein the first lipo-coacervate further comprises a saturated lipid and/or cholesterol.

Aspect 18. The method of aspect 17, wherein the saturated lipid comprises 18:0 PG (DSPG).

Aspect 19. The method of aspect 18, wherein the first lipo-coacervate comprises cholesterol, DOPC and DSPG.

Aspect 20. The method of any one of aspects 15-19, wherein the second lipo-coacervate comprises cholesterol and at least one saturated lipid selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

Aspect 21. The method of aspect 20, wherein the second lipo-coacervate comprises cholesterol, DPPC and DSPG.

Aspect 22. The method of any one of aspects 9-21, wherein the coacervate, the first coacervate and/or the second coacervate comprises poly(ethylene arginyl aspartate diglyceride) (PEAD) and heparin.

Aspect 23. The method of any one of aspects 1-22, wherein the wound is a dermal wound or a mucosal wound.

Aspect 24. The method of aspect 23, wherein the dermal wound comprises a laceration, a puncture wound, an abrasion, a surgical wound, a burn, an ulcer or a pressure sore.

Aspect 25. The method of aspect 23, wherein the mucosal wound is in the nose, mouth, rectum, anus, vagina or lung.

Aspect 26. The method of any one of aspects 1-25, wherein administration comprises topical administration.

Aspect 27. The method of any one of aspects 1-25, wherein administration comprises injection at or near the site of the wound.

Aspect 28. A controlled delivery composition for treating a wound, comprising:
a therapeutically effective amount of a first agent that promotes wound closure selected from heparin binding EGF-like growth factor (HB-EGF), tenascin-C (TNC), a growth factor, a matricellular protein, interleukin (IL)-1, IL-2 and IL-4; and
a therapeutically effective amount of a second agent that inhibits scarring selected from decorin (DCN), a CXCR3 ligand, collagen type I and IL-10,
wherein the controlled delivery composition permits rapid release of the first agent and delayed release of the second agent.

Aspect 29. The controlled delivery composition of aspect 28, wherein:
the growth factor is selected from epidermal growth factor (EGF), vascular endothelial growth factor (VEGF), a member of the fibroblast growth factor (FGF) family, transforming growth factor (TGF)-«, amphiregulin, and platelet-derived growth factor (PDGF);
the matricellular protein is selected from secreted protein acidic and cysteine rich (SPARC), thrombospondin, laminin B1, and collagen type III; and/or
the CXCR3 ligand is selected from CXCL4, CXCL9, CXCL10 and CXCL11.

Aspect 30. The controlled delivery composition of aspect 28 or aspect 29, comprising a hydrogel, wherein the hydrogel comprises:
the first agent; and
the second agent encapsulated in a coacervate,
wherein upon administration of the controlled delivery composition, the first agent is released from the hydrogel prior to release of the second agent from the coacervate.

Aspect 31. The controlled delivery composition of aspect 28 or aspect 29, wherein the controlled delivery composition comprises:
a first coacervate comprising the first agent;
a second coacervate comprising the second agent, wherein the second coacervate is encapsulated by lipids, thereby forming a lipo-coacervate,
wherein upon administration of the controlled delivery composition, the first agent is released from the first coacervate prior to release of the second agent from the lipo-coacervate.

Aspect 32. The controlled delivery composition of aspect 31, wherein the lipids of the lipo-coacervate comprise:
cholesterol; and
at least one unsaturated lipid and/or at least one saturated lipid.

Aspect 33. The controlled delivery composition of aspect 32, wherein:
the at least one unsaturated lipid is selected from 18:1 (Δ9-Cis) PC (DOPC), 18:1 (A9-Cis) PG (DOPG), 18:1 (A9-Cis) PE (DOPE), 16:0-18:1 PE (POPE), 16:0-18:1 PC (POPC), 16:0-18:1 PS (POPS) and 18:1 DGS-NTA (Ni); and/or
the at least one saturated lipid is select from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

Aspect 34. The controlled delivery composition of aspect 32 or aspect 33, wherein the lipids of the lipo-coacervate comprise cholesterol, DOPC and DSPG.

Aspect 35. The controlled delivery composition of aspect 32 or aspect 33, wherein the lipids of the lipo-coacervate comprise cholesterol, DPPC and DSPG.

Aspect 36. The controlled delivery composition of aspect 28 or aspect 29, wherein the controlled delivery composition comprises:
a first coacervate comprising the first agent, wherein the first coacervate is encapsulated by lipids, thereby forming a first lipo-coacervate; and
a second coacervate comprising the second agent, wherein the second coacervate is encapsulated by lipids, thereby forming a second lipo-coacervate,
wherein upon administration of the controlled delivery composition, the first agent is released from the first lipo-coacervate prior to release of the second agent from the second lipo-coacervate.

Aspect 37. The controlled delivery composition of aspect 36, wherein the first lipo-coacervate comprises cholesterol and at least one unsaturated lipid selected from 18:1 (Δ9-Cis) PC (DOPC), 18:1 (Δ9-Cis) PG (DOPG), 18:1 (A9-Cis) PE (DOPE), 16:0-18:1 PE (POPE), 16:0-18:1 PC (POPC), 16:0-18:1 PS (POPS) and 18:1 DGS-NTA (Ni).

Aspect 38. The controlled delivery composition of aspect 37, wherein the first lipo-coacervate further comprises a saturated lipid.

Aspect 39. The controlled delivery composition of aspect 38, wherein the saturated lipid comprises 18:0 PG (DSPG).

Aspect 40. The controlled delivery composition of aspect 39, wherein the first lipo-coacervate comprises cholesterol, DOPC and DSPG.

Aspect 41. The controlled delivery composition of any one of aspects 36-40, wherein the second lipo-coacervate comprises cholesterol and at least one saturated lipid selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

Aspect 42. The controlled delivery composition of aspect 41, wherein the second lipo-coacervate comprises cholesterol, DPPC and DSPG.

Aspect 43. The controlled delivery composition of any one of aspects 30-42, wherein the coacervate, the first coacervate and/or the second coacervate comprises poly(ethylene arginyl aspartate diglyceride) (PEAD) and heparin.

### EXAMPLES

The following examples are provided to illustrate particular features of certain aspects of the disclosure, but the scope of the claims should not be limited to those features exemplified.

### Example 1: Background, Materials and Methods

This example provides the background, materials and methods for Examples 2-5.

### Background

Mesenchymal stem cells/multipotent stromal cells (MSCs) have emerged as a promising candidate therapy for chronic skin wounds due to their genuine involvement in the early phases of routine wound healing. Upon injury, endogenous bone marrow MSCs home to the site of injury and secrete paracrine signals to modulate the inflammation process and promote angiogenesis (Sylakowski et al., Am J Pathol. July 2020; 190 (7): 1370-1381; Bradshaw et al., Curr Pathobiol Rep. June 2018; 6 (2): 109-115; Rustad et al., Adv Wound Care (New Rochelle). August 2012; 1 (4): 147-152.; Li et al., Front Med. March 2011; 5 (1): 33-39). Numerous pre-clinical and clinical studies have looked at the utility of MSCs and the enhanced benefits of their use in treating a myriad of chronic wound conditions (Huang et al., Tissue Eng Part B Rev. December 2020; 26 (6): 555-570). However, a significant challenge limiting overall MSC therapeutic potential is the poor survival rate and engraftment post-transplantation into the wound bed, with up to 90% of the introduced MSCs being lost within the first three days (Sylakowski et al., Am J Pathol. July 2020; 190 (7): 1370-1381; Hu et al., J Thorac Cardiovasc Surg. April 2008; 135 (4): 799-808). The chronic wound microenvironment is a hostile culmination of damaged extracellular matrix (ECM), cellular debris, hyper inflammation, and an impaired vasculature (Hu et al., Plast Surg Int. 2015; 2015:383581; Rodrigues et al., Stem Cell Res Ther. Oct. 26, 2010; 1 (4): 32). These factors induce MSC death through various mechanisms, including anoikis, ischemic insult, or increased signaling from death cytokines (Sylakowski et al., Am J Pathol. July 2020; 190 (7): 1370-1381; Song et al., Expert Opin Biol Ther. March 2010; 10 (3): 309-319).

The present disclosure addresses this problem by taking cues from the normal wound healing response and utilizing a naturally occurring matricellular protein called Tenascin-C (TNC). In unwounded skin, TNC is expressed at negligible levels in the papillary dermis just beneath the basement membrane (Lightner et al., J Cell Biol. June 1989; 108 (6): 2483-2493). Shortly after injury, TNC is significantly increased at all levels of the skin: in the epidermis through the secretion of epidermal keratinocytes (Latijnhouwers et al., J Invest Dermatol. May 1997; 108 (5): 776-783), at the epidermal-dermal junction just under the basal lamina (Filsell et al., Br J Dermatol. April 1999; 140 (4): 592-599), as well as throughout granulation tissue within the dermis (Mackie et al., J Cell Biol. December 1988; 107 (6 Pt 2): 2757-2767). TNC is a six-armed glycoprotein composed of four main domains: the TNC assembly domain, the epidermal growth factor-like (EGF-L) repeat domain, the fibronectin type III (FNIII) domain, and a fibrinogen globe domain. The 14.5 EGF-like repeats are found on each arm of the TNC hexamer (Midwood and Orend, J Cell Commun Signal. December 2009; 3 (3-4): 287-310). These EGF-like repeats bind to the epidermal growth factor receptor (EGFR) through low affinity/high avidity interactions, allowing for the sequestering of the EGFR to the cell membrane preventing its internalization and degradation (Iyer et al., J Cell Physiol. June 2007; 211 (3): 748-758; Swindle et al., J Cell Biol. Jul. 23, 2001; 154 (2): 459-468). TNC promotes MSC survival in the face of Fas ligand-induced cell death by binding and sequestering the EGFR, resulting in prolonged activation of the AKT and ERK pro-survival signaling pathways (Rodrigues et al., Stem Cell Res Ther. Oct. 26, 2010; 1 (4): 32; Rodrigues et al., Tissue Eng Part A. September 2013; 19 (17-18): 1972-1983). MSCs in a TNC-based polymer system in vivo, were able to survive out to 21 days post-transplantation within the wound bed (Yates et al., Cell Transplant. Jan. 24, 2017; 26 (1): 103-113). The studies disclosed herein investigate the use of MSCs to promote healing.

This study assesses whether the benefit of TNC is not just to promote MSC survival, but also to improve the functional capacity of the wound bed. One of the significant hurdles within the chronic wound bed that newly transplanted cells must encounter is ischemia. Therefore, MSCs cultured on TNC were first subjected to in vitro hypoxia/nutrient deprivation (H/ND) growth conditions and assessed for survival advantages. Next, TNC-supported MSC culture conditions subjected to H/ND were evaluated for angiogenic influence on endothelial cells, as reestablishing new vasculature is necessary to overcome ischemia and progress the wound healing process forward. The last set of experiments were designed to determine how TNC-MSC systems would translate in vivo. Since chronic wounds have a characteristically elevated quantity of metalloproteinases (MMPs) and other protein degrading enzymes compared to normal healing wounds (Frykberg and Banks, Adv Wound Care (New Rochelle). Sep. 1, 2015; 4 (9): 560-582), a protein delivery system called coacervate was used to help protect TNC against early degradation post-implantation. This system is an injectable in vivo delivery vehicle that uses a positively charged synthetic biodegradable poly(ethylene arginyl aspartate diglyceride) (PEAD) and a negatively charged heparin to form a 3-dimensional coacervate that envelopes around the protein cargo of choice (Johnson and Wang, J Control Release. Mar. 10, 2013; 166 (2): 124-129; Johnson and Wang, Wound Repair Regen. July-August 2015; 23 (4): 591-600; Park et al., Acta Biomater. May 2019; 90:179-191; Li et al., Stem Cells Transl Med. September 2013; 2 (9): 667-677). The animal model used for this study is an impaired wound healing CXCR3^{-/-} mouse model. This mouse model exhibits a delayed healing response within the dermal and epidermal layers of the skin, leading to an immature dermal matrix, a weakened basement membrane, and hypercellularity (Huen and Wells, Adv Wound Care (New Rochelle). December 2012; 1 (6): 244-248; Yates et al., Wound Repair Regen. January-February 2009; 17 (1): 34-41; Yates et al., Am J Pathol. August 2007; 171 (2): 484-495). It was hypothesized that TNC would enhance MSC angiogenic efficacy and thus further contribute to better wound healing outcomes in the delayed wound healing mouse model.

### Cell Culture

MSC Cell Culture—One immortalized bone marrow mesenchymal stem cell (IHMSC) and three primary bone marrow-derived MSC (PrhMSC) lines were used. The IHMSC cell line was a human bone marrow-derived cell line that what immortalized by using human telomerase reverse transcriptase (Shima et al., Biochem Biophys Res Commun. Feb. 2, 2007; 353 (1): 60-66). IHMSCs were cultured using the following proliferation media formulation: DMEM with L-Glutamine, 1 g/L Glucose and Sodium Pyruvate from Corning (Cat. No. 10-014-CV), supplemented with 10% FBS (Cat. No. 100-106, Gemini Bio-Products), 1 mM sodium pyruvate, 1 mM L-glutamine, 1 μM non-essential amino acids, and 100 units per mL penicillin-streptomycin. PrhMSCs were obtained from the repository at the Darwin Prockop laboratory at Texas A&M University, a National Institute of Health-funded stem cell repository. PrhMSCs were cultured using the following proliferation media formulation: α-MEM (Cat. No. 15-012-CV, Corning) supplemented with 16.5% FBS (Cat. No. S11550H, Atlanta Biologicals), 2 mM L-glutamine, and 100 units per mL penicillin/streptomycin.

For hypoxia and nutrient deprivation conditions (H/ND) to mimic ischemia in vitro, MSCs were expanded and seeded near confluence onto one of three treatment coatings (Plastic, Col-1 or TNC+Col-1) and cultured in their fully supplemented DMEM for one day at ambient air conditions (37° C. at 5% CO2 and 21% O₂) to stabilize after passaging. Meanwhile, basal DMEM and α-MEM without supplementation or FBS were placed into BioSpherix incubators at 1% oxygen to acclimate the media 24 hours prior to adding to the cells. Upon the start of H/ND experiments of MSCs, the complete culture medias were aspirated, the plates were washed twice with PBS to remove residual media, and pre-equilibrated H/ND media was added to the MSCs. The MSCs were then placed into the BioShperix incubators at 1% oxygen. At no point in the culture of cells at 1% were the cells exposed to ambient oxygen conditions, as microscopes to monitor cells were also contained in the BioSpherix chamber. Positive confirmation of hypoxia on MSCs was confirmed through a live cell green hypoxia dye (Cat. No. SCT033, EMD Millipore) as instructed by the company's protocol at the beginning stages of each H/ND experiment (FIG. 6).

Endothelial Cell Culture—Immortalized Human Microvascular Endothelial Cells (HMEC-1) were obtained through ATCC (Cat. No. CRL-3242; Ades et al., J Invest Dermatol. December 1992; 99 (6): 683-690.) and cultured using the following proliferation media formulation: MCDB131 basal media without L-glutamine (Fisher Scientific) supplemented with 10% FBS, 10 ng/mL epidermal growth factor, 1 μg/mL hydrocortisone, and 10 mM glutamine. HMEC-1 were thawed and expanded up to passage 3 in proliferation media, then transitioned to either IHMSC or PrhMSC media conditions over a two-week period for functional endpoint experiments. By Passage 6 HMEC-1s were fully transitioned to MSC media conditions and used for cell migration and cord formation studies.

Coating Culture Plates with Col-1 and TNC

Cell culture plate coating procedures and concentrations used for Tenascin-C (TNC) (Cat. No. CC065, EMD Millipore) and type 1 rat tail collagen (Col-1) (Cat. No. 354236, BD Biosciences) were as previously described (Rodrigues et al., Tissue Eng Part A. September 2013; 19 (17-18): 1972-1983). In short, cell culture dishes were coated for 12 hours at 37° C. with 1 μg/cm² Col-1, or 1 μg/cm² Col-1 and 1 g/cm² TNC diluted in PBS. After the incubation period, the PBS was aspirated, and the coated surfaces were placed under UV light for 30 minutes before seeding any cells. The Col-1 was required for the TNC-coated plates, as TNC is anti-adhesive and requires additional integrin-binding epitopes as provided by the collagens (Rodrigues et al., Tissue Eng Part A. September 2013; 19 (17-18): 1972-1983).

### Apoptosis Assessment

Two methods determined cell death. In the first method, flow cytometry combined with an Annexin V and propidium iodide (PI) kit (Cat. No. V13242, Thermo Fisher Scientific) were performed as per manufacturers' instruction. In brief, MSCs cultured under H/ND growth conditions on either plastic, Col-1, or TNC+Col-1 were harvested by collecting adherent cells and floating cells in the supernatant. The floating and adherent cells were combined and stained for PI and Annexin V for 15 minutes at room temperature. An appropriate volume of 1× annexin-binding buffer and counting beads (Cat. No. C36950, Thermo Fisher Scientific) were added and the tubes placed on ice. Samples were run through a BD FACSCanto II flow cytometer and analyzed using FlowJo_V10. An unstained control for each cell type was used to determine positive and negative stained cells and counting beads were used to determine the absolute number of cells per treatment condition for quantification.

For the second method, MSCs were seeded onto 6-well plates for each condition (plastic, Col-1, or TNC+Col-1) and subjected to H/ND. Old media was removed, PBS with CellEvent caspase 3/7 green detection reagent (Cat. No. C10423, Thermo Fisher Scientific), and 5% FBS was added to each well and left to incubate for 30 minutes at 37° C. Cells were imaged and quantitated using ImageJ, where all images were converted to 8-bit format, and the threshold was reduced to highlight the green punctae in culture, using the same thresholding for all analyzed images. Particle analysis in ImageJ was set to detect fluorescent points with a radius greater than 50 pixels to exclude debris and background fluorescence. The number of caspase 3/7 positive and negative cells were calculated and expressed as a percentage of Caspase 3/7 positive staining. This second method procedure was also used to determine the survival time course during exposure to H/ND, but instead of using caspase 3/7, PI from the provided Annexin V/PI kit was initially used.

### Preparation of Conditioned Media

Conditioned media (CM) was harvested at 96-hour time point from the four MSC lines cultured under H/ND growth conditions on either plastic, Col-1, or TNC+Col-1. CM was then centrifuged for 10 min at 10,000 rpm to remove cells and cellular debris. The supernatant was collected and frozen for downstream assays.

### Cord Formation Assay

Cord formation assays were performed using μ-slide angiogenesis glass-bottom slides (Cat. No. 81507, Ibidi) as per manufacturers' instruction. In brief, growth factor reduced Matrigel (Cat. No. 356231, BD Biosciences) was seeded in the bottom well of the ibidi angiogenesis plates and allowed to polymerize for 30 minutes at 37° C. While the gel was polymerizing, low passage fully transitioned HMEC-1 cells were trypsinized, counted, and mixed with a ratio of 50% basal media and 50% freshly thawed CM for each treatment condition. Basal media alone was used as the negative control, while basal media with 10% FBS was used as the positive control. Cells were then seeded into the Ibidi angiogenesis chamber at 2×10⁵ and placed into a 37° C. incubator with ambient growth conditions. Cords were allowed to form over the next 6 hours before being quantified using ImageJ Angiogenesis Analyzer (Carpentier et al., Sci Rep. Jul. 14, 2020; 10 (1): 11568) and expressed as a total number of cords, total cord length, the total number of meshes, and total mesh area. Cords are the sum of segments (elements bordered by two junctions) and branches (elements bordered by a junction and one extremity); and a mesh is defined as an area enclosed by segments (Carpentier et al., Sci Rep. Jul. 14, 2020; 10 (1): 11568).

### Scratch Assay

Cell migration was assessed using the wound healing (or scratch) assay. HMEC-1 were seeded into 24 well plates and allowed to grow to confluence. Upon reaching confluence, the cells were washed in PBS and then cultured in basal media containing 1% dialyzed FBS for 24 hours to limit proliferation during the study. The next day using a 1000 mL pipette tip, scratches were made down the middle of each plate to create a denuded area. Each well was washed twice with PBS to remove cellular debris after the scratch was made. A mixture of 50% basal media with 1% FBS and 50% CM from each treatment condition were added to their respective wells. Basal media alone was used as the negative control while basal media with 10% FBS was used as the positive control. Images were acquired at 0 and 24 hours, and the area unoccupied by the migrating cells was determined using Image J and expressed as % wound closure.

### Cytokine Measurement

MSCs were seeded onto 6-well plates for each condition (plastic, Col-1, or TNC+Col-1) and subjected to H/ND. At 96 hours, CM was collected, centrifuged to remove cellular debris, and frozen until ready to use. To determine angiogenic factors in the MSC CM, a semi-quantitative human angiogenesis antibody array (Cat. No. AAH-ANG-1000-2, RayBiotech) was used according to the instructions as outlined in their protocol. In brief, 1 mL of CM from each MSC treatment group was placed on a specialized PVDF membrane with specific angiogenic capture antibodies and incubated overnight at 4° C. A series of washes and conjugation of biotinylated antibodies were performed following the incubation period. According to the kit's protocol, membranes were washed 3 times more and detected using labeled streptavidin and chemiluminescence. Radiographs were processed and analyzed using ImageJ Dot Blot analyzer plug-in.

### RT-PCR Analysis

MSCs were seeded onto 6-well plates for each condition (plastic, Col-1, or TNC+Col-1) and subjected to H/ND. At 48 hours, MSC treatment conditions were washed with PBS, trypsinized and pelleted, and RNA was isolated using RNeasy kit (Cat. No. 74004, Qiagen). Quality and concentration analysis of RNA samples was assessed using a nanodrop instrument and stored at −80° C. Next, the Isolated RNA samples were synthesized into cDNA using The RT2 First Strand c-DNA Synthesis Kit from Qiagen (Cat. No. 330404, Qiagen). MSC angiogenic and wound healing genes were assessed using Qiagen RT2 Profiler Array System (Cat. No. PAHS-024ZA-12 and PAHS-121ZA-12, Qiagen). RT-PCR was performed on the Stratagene Mx3000P Real-Time PCR System with the Qiagen RT2 SYBR Green qPCR Master Mix (Cat. No. 330523, Qiagen). Samples were analyzed using the ΔΔCT method plus or minus standard error and expressed in terms of fold regulation. MSCs grown on Col-1 and TNC+Col-1 were normalized back to MSCs grown on plastic, and GAPDH was used as the reference gene. Fold-regulation is similar to fold change in that positive values greater than one indicate up-regulation; however, fold-change values less than one that indicates down-regulation have now been transcribed to the negative inverse. For this analysis, 2 and −2 were used as fold regulation cut-off numbers for anything of importance; and CT values more than 32 were seen as likely biologically irrelevant.

### Animal Model

C57BL/6J CXCR3−/− mice were generated as previously described (Hancock et al., J Exp Med. Nov. 20, 2000; 192 (10): 1515-1520). The C57BL/6J CXCR3^{-/-} mice were then bred to FVB strain mice for at least 20 generations to yield germline transmission of the targeted allele and create CXCR3 devoid mice on the FVB background. For this study, CXCR3-4-female mice were bred with CXCR3-males, and all offspring were screened for genotype through PCR before use. Serological analyses did not detect blood-borne pathogens or evidence of infection. Mice were housed in individual cages after wounding to limit fighting and further scarring and maintained under a 12-hour light/dark cycle.

### Mouse Wounding

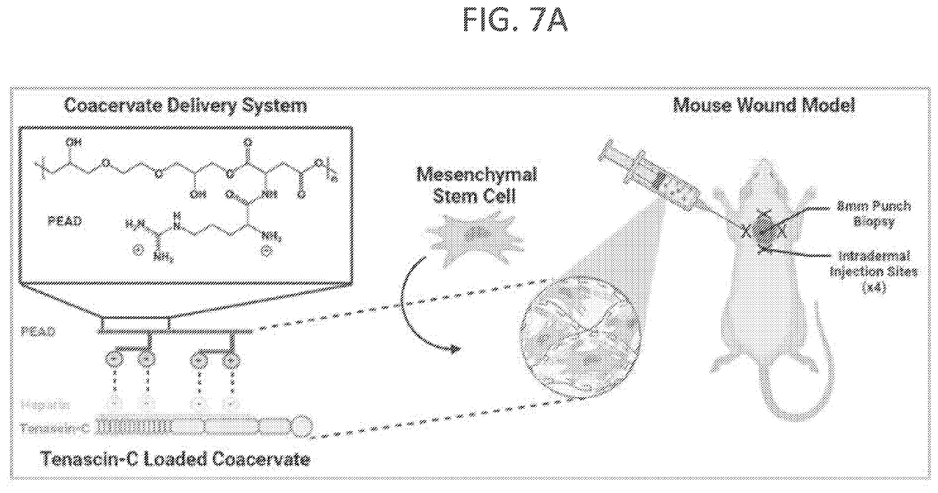

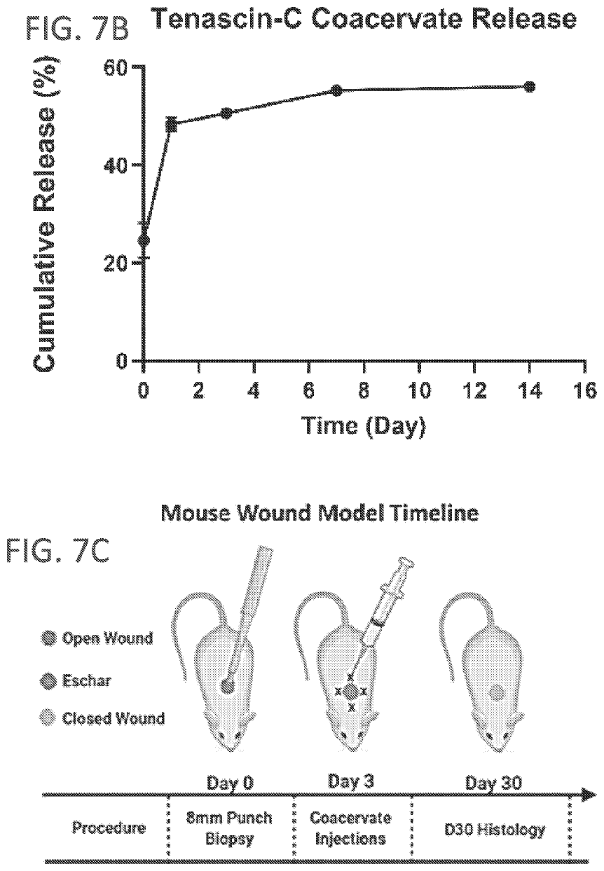

Male and female mice (8 to 9 months of age and weighing approximately 25 g) were anesthetized with an intraperitoneal injection containing ketamine (75 mg/kg) and xylazine (5 mg/kg). The backs were cleaned, shaved, and sterilized with betadine solution. Full-thickness wounds were performed using an 8 mm diameter punch biopsy through the epidermal and dermal layers along one side of the dorsal midline of the mouse (FIG. 7A). The contralateral uninjured skin served as unwounded control skin.

### Coacervate Preparation and Mouse Injections

PEAD was synthesized as previously described (Chu et al., J Control Release. Mar. 10, 2011; 150 (2): 157-163). Stock solutions of PEAD and heparin (10 mg/mL) were prepared in 0.9% saline solution using a 0.22 μm sterile syringe filter. Human Tenascin-C purified protein (Cat. No. CC065, EMD Millipore) was reconstituted to 300 μg/mL and mixed with heparin stock solution at their respective predetermined working dose of 10 μg per animal. The heparin/TNC solution was then mixed with the PEAD stock solution to form protein-encapsulated coacervates at a final ratio of 1:100:500 mass ratio. Coacervation was determined by the rapid formation of a turbid solution once all components were added. The prepared coacervate mixture was then mixed with a prepared cell suspension of 50×10⁴ IHMSCs per mouse for a total injection volume of 300 μL per mouse. Intradermal Injections were administered 72 hours post wounding. Each mouse received 300 μL of one of five treatment options: CO only, CO+TNC, CO+MSC, TNC+MSC, or CO+TNC+MSC, through a series of 4 injections of 75 μL around the wound bed to avoid tissue trauma (FIG. 7A). Each treatment group comprised of 4 animals each.

### Coacervate Release

Coacervate was prepared as stated above to create three independent samples for coacervate containing TNC. Samples were incubated at 37° C. for 14 days, and the supernatant was harvested on days 0, 1, 3, 7, and 14 through pelleting the coacervates by centrifugation (12,100 g for 10 min). Supernatants were analyzed using pre-coated enzyme-linked immunosorbent assay (ELISA) kits (Cat. No. ab213831, Abcam), according to the manufacturer's specifications (FIG. 7B).

### Histological Analyses

Wound bed biopsies were collected after euthanasia at day 30 using a 12 mm punch biopsy and were immediately fixed in 10% buffered formalin. Samples were then paraffin embedded (FFPE), sectioned into slides (6 μm), and stained with H&E and Masson's Trichrome (MT). Blank slides were also made for downstream Picrosirius Red (PSR) staining as directed by the manufacturer's protocol (Cat. No. 150681, Abcam) and for follow up immunohistochemistry and immunofluorescent stains.

Wound healing score assessment-Histopathological examination of mouse tissues was performed in a double-blind approach among three investigators, and their scores averaged. Qualitative assessments were based on epidermal and dermal maturation as outlined in previously established protocols (Yates et al., Wound Repair Regen. January-February 2009; 17 (1): 34-41; Yates et al., Am J Pathol. August 2007; 171 (2): 484-495; Yates et al., Am J Pathol. September 2008; 173 (3): 643-652). In short, the samples were scored on a scale of 0 to 4 for both epidermal and dermal layers of tissue. For epidermal maturation scoring: 0=no epidermal migration, 1=partial epidermal migration, 2=complete epidermal migration, 3=partial keratinization and an intact basement membrane, 4=complete keratinization and normal epidermis. For dermal maturation scoring: 0=no healing, 1=inflammatory infiltration, 2=granulation tissue present-fibroplasias and angiogenesis, 3=collagen deposition replacing granulation tissue>50%, 4=complete replacement of granulation tissue and complete healing.

Skin thickness measurements—H&E and MT sample images were analyzed using ImageJ line measurement tool to measure the thickness of the epidermal and dermal layers for each mouse specimen. The epidermal layers were measured from the stratum basale to the stratum granulosum (excluding the stratum corneum), and the dermal layer was measured from the top of the papillary layers to the bottom of the reticular layer. A total of 6 measurements per biological sample were scored for each layer.

Collagen quantification—Polarized PSR images were quantified using ImageJ version 1.53K. Each image was split into red, green, and blue channels, from which the red and green channels were selected. The isolated red and green channels then underwent thresholding and follow-up measurements to determine the total staining area for each collagen type. PSR birefringence under polarized light reveals tightly packed thick and long fibrils of type 1 collagen as either bright red-orange intense birefringence in tissue and thin short loose fibrils as yellow-green (Coelho et al., An Bras Dermatol. June 2018; 93 (3): 415-418). The overall ratio of type 1 collagen to type III collagen was analyzed by comparing the representative staining results for either collagen type back to the original image for total collagen content.

Collagen orientation and alignment—Polarized PSR images were quantified using ImageJ plug-in: OrientationJ version 2.0.5. Visual directional analysis using the color survey tool was performed with the following parameters: Hue is set for orientation, Saturation is set for coherency, and brightness is set to the original image (Puspoki et al., Adv Anat Embryol Cell Biol. 2016; 219:69-93). The OrientationJ distribution tool was used to create the distribution of orientation histograms to determine where patterns of alignment were within the samples (Rezakhaniha et al., Biomech Model Mechanobiol. March 2012; 11 (3-4): 461-473). The OrientationJ Dominant Direction tool was used to determine the quantitative orientation measurement for coherency across the whole image (Fonck et al., Stroke. July 2009; 40 (7): 2552-2556).

Immunohistochemistry staining—Skin sample tissue sections (6 μm) were rehydrated and placed into citrate acid buffer at 95° C. for 10 minutes and cooled to room temperature for 1 hour. Samples were then washed in phosphate-buffered saline (PBS) and quenched with 3% hydrogen peroxide. Following additional washes in PBS, samples were blocked in 20% goat serum for 1 hour and subsequently incubated with primary antibodies diluted in blocking buffer overnight at 4° C. Primary antibodies were at the following dilutions: CD31 (1:50, Cat. No. ab28364, Abcam). After primary antibody incubation, samples were washed in PBS and incubated in goat anti-rabbit HRP secondary antibody (1:2000, Cat. No. ab205718, Abcam) for 1 hour at room temperature. After final antibody incubations, samples were washed in PBS and then treated with ABC reagent (Cat. No. PK-6100, Vector Laboratories) for 30 minutes. Rewashed with PBS and developed with DAB reagent kit 27 (Cat. No. SK-4100, Vector laboratories), counterstained with H&E, clarified (Richard Alan Scientific), and dehydrated in ethanol and xylene. Samples were mounted (Cat. No. SP15-500, Fisher Scientific) and imaged using an Olympus Provis microscope. Image analysis was performed using Image J Colour Deconvolution 2 plug-in to acquire DAB positive staining area. The total DAB positive area was compared back to the total area of the original image to get a final percentage.

### Statistical Analysis

Data for the in vitro cell survival and cytokine array are presented as the mean±standard deviation, whereas the cord formation and cell migration assays are presented as the mean±s.e.m. All in vitro studies were performed using four biological replicates with at least 2 technical replicates. Results from animal wound healing score assessment and PSR staining assessment are expressed as mean±SD with at least a minimum of four mice per treatment group. Histological quantifications for skin layer thickness and immunohistological stains (CD31) were performed on five microscopic fields of each specimen and reported as mean±s.e.m. One-way ANOVA determined statistical analysis between treatment groups for all experiments except for the survival time course for which two-way ANOVA was used to distinguish significance. Both ANOVA analyses were followed by posthoc Tukey HSD analysis using GraphPad Prism 9 software (GraphPad Software, San Diego, CA). Significance was claimed for P<0.05. Significance is represented in all figures with symbols denoting *P<0.05, **P<0.01, ***P<0.001, and ****P<0.0001

### Example 2: Tenascin-C Protects MSCs Under Ischemic Conditions In Vitro

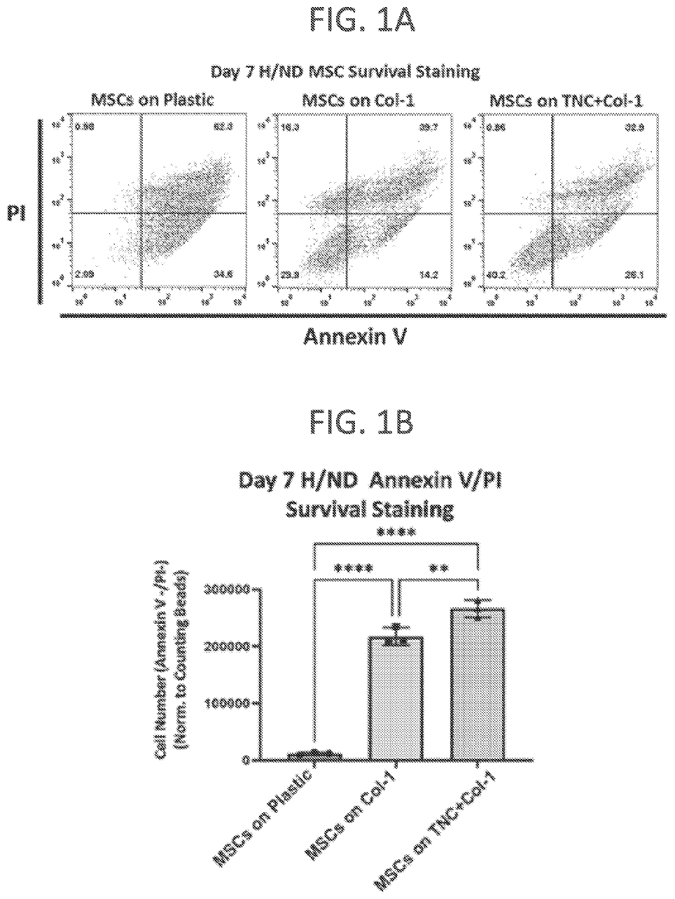

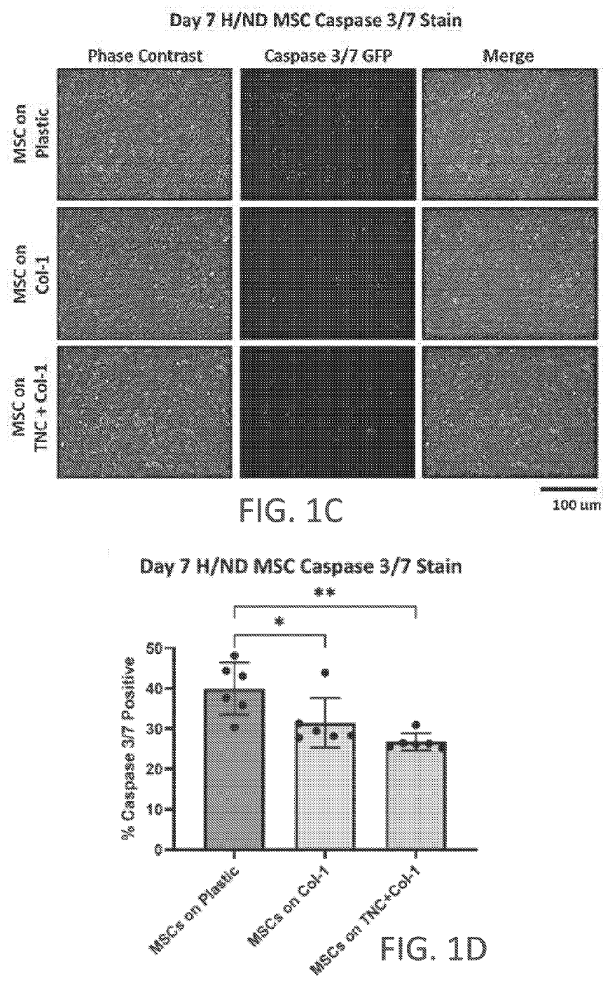

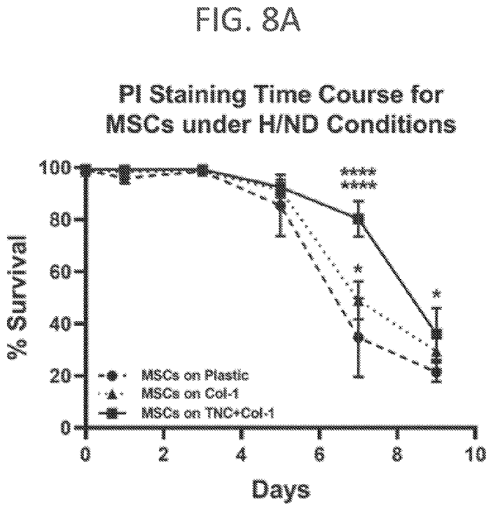

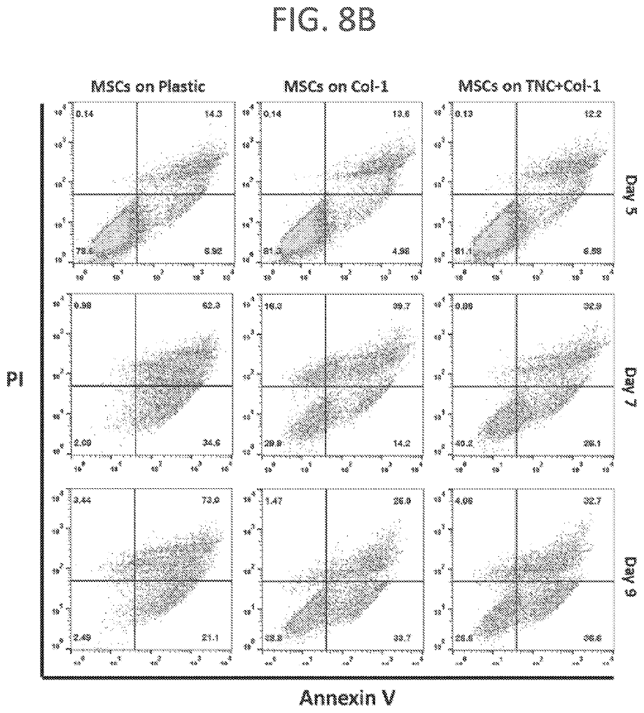

One of the major obstacles facing MSC survival and engraftment within chronic wounds is ischemia. TNC has been shown to protect MSCs against inflammatory death signals (Rodrigues et al., Tissue Eng Part A. September 2013; 19 (17-18): 1972-1983), and the current study aims to determine whether this matricellular protein also ameliorates the ischemia due to lack of angiogenesis. An ischemic environment was created in vitro using a Biospherix growth chamber (Nuschke et al., Stem Cell Res Ther. Dec. 1, 2016; 7 (1): 179). A cell survival time course was made using Propidium Iodide (PI) as a marker for dead cells. A significant difference was observed at day 7 between treatment groups (MSC on plastic, MSC on Col-1, or MSC on TNC+Col-1) under H/ND conditions (FIG. 8A). MSCs cultured on TNC+Col-1 had much lower PI incorporation when compared to MSCs cultured on either plastic (34%) or Col-1 alone (48%). Additionally, MSCs cultured on Col-1 showed better survival over MSCs cultured on plastic, though not to the degree of TNC+Col-1. The survival changes around Day 7 were further analyzed using flow cytometry with a combination of PI and Annexin V cell death markers. In this system, PI can detect necrotic or late apoptotic cells through the loss in integrity of the plasma membrane, and Annexin V signals early apoptosis through detection of plasma membrane flipped phosphatidylserine as well as late apoptosis. A snapshot of this transition at day 7 revealed that only 2.09% of the MSCs on plastic did not transition through apoptosis (PI−/Annexin V−); compared to the survival rates of MSCs on Col-1 at 29.9% or MSCs on TNC+Col-1 at 40.2%; the survival on TNC+Col-1 was significantly higher than Col-1 alone (FIGS. 1A, 1B and 8B).

To confirm the flow cytometry data results, an additional set of staining experiments were performed with a live cell green caspase 3/7 stain and apoptosis was quantified on day 7 (FIGS. 1C, 1D). Once again, MSCs cultured on plastic were significantly limited in survivability, with mean positive staining for caspase 3/7 of 39%, while MSCs cultured on Col-1 or TNC+Col-1 had lower mean caspase 3/7 staining rates of 31% and 27%, respectively. With caspase 3/7, the difference between the Col-1 and TNC+Col-1 survival rates did not reach statistical significance; this could be due to the loss of dead cells floating off the bottom of the plate, where these were captured and quantified in the flow analysis. The data indicate that MSCs seeded on TNC+Col-1 have significantly higher protection against the ischemic microenvironment than MSCs cultured on plastic alone and a slightly higher survival rate than MSCs cultured on Col-1.

### Example 3: Conditioned Media from MSCs Cultured on Tenascin-C Improves Endothelial Cord Formation and Motility

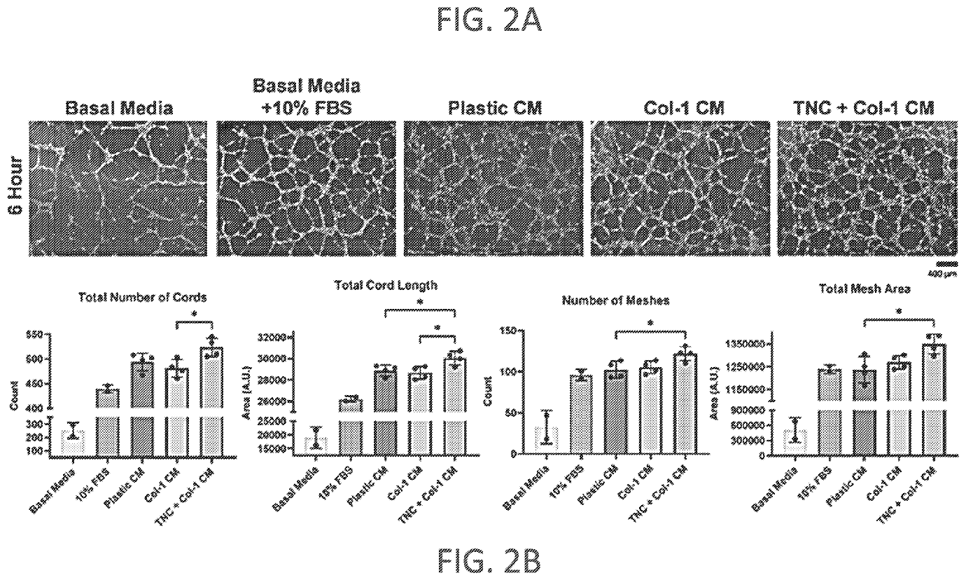

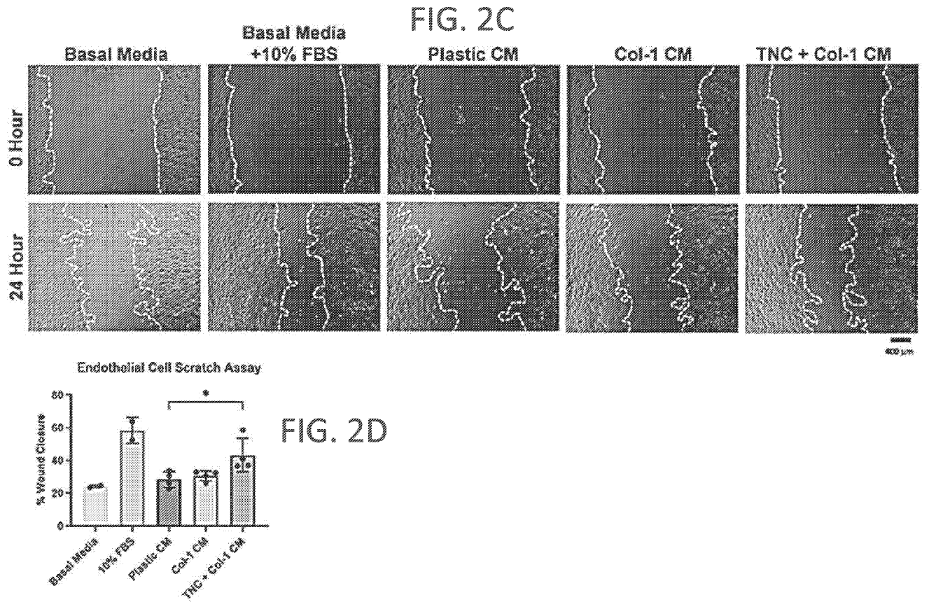

TNC can contribute to the wound angiogenic response by enhancing MSC-produced angiogenic factors. To assess TNC's influence on MSC's ability to modulate the ischemic wound environment and improve vascularization, studies were performed to evaluate the angiogenic effects of MSC conditioned media (CM) on endothelial cells in vitro. CM was harvested from MSCs cultured on either plastic, Col-1, or TNC+Col-1 under H/ND conditions and used with HMEC-1 plated on a reduced growth factor Matrigel surface to commence cord formation (FIG. 2A). Overall, CM from all three treatment groups performed equal to or better than the positive control of basal media+10% FBS across all metrics (FIG. 2B). However, the CM from MSC cultured on TNC+Col-1 scored significantly higher than MSCs cultured on plastic for total cord length, the total number of meshes, and total mesh area. Additionally, CM from MSCs cultured on TNC+Col-1 had significantly longer length and a higher number of cords compared to CM from MCSs cultured on Col-1 alone, and trended higher on the other matrices though the increases did not reach statistical significance.

After assessing the potential angiogenic influences, further studies sought to examine the cell motility effects of the different culture conditions through a wound-healing/scratch assay. CM harvested from the varying MSC culture conditions (plastic, Col-1, TNC+Col-1) were placed into confluent HMEC-1 wells with a denuded strip in the center to allow for cell migration. Images were taken at 0 and 24 hours post addition of the CM, and cell migration into the denuded area was measured (FIGS. 2C, 2D). Unlike in the cord formation assay, none of the MSC treatment groups outperformed the positive control group this time. The basal media negative control had a wound closure rate of 23%, while CM from both plastic and Col-1 scarcely outperformed at 28% and 30%. CM harvested from TNC+Col-1 performed significantly better with a wound closure rate of 43%. Overall, TNC's interactions with MSCs have a pro-angiogenic effect.

### Example 4: Assessment of Culture Conditions on MSC Paracrine Activity and Gene Expression During H/ND Exposure

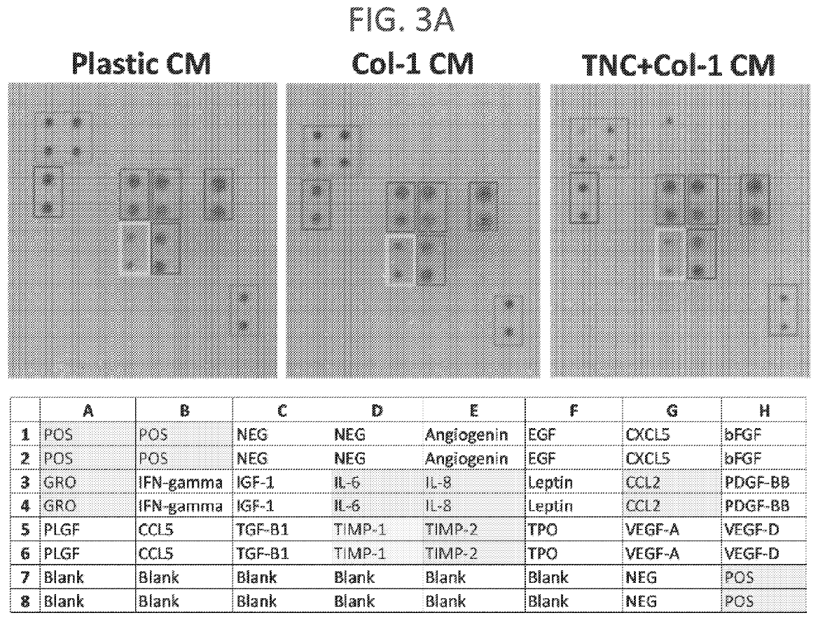

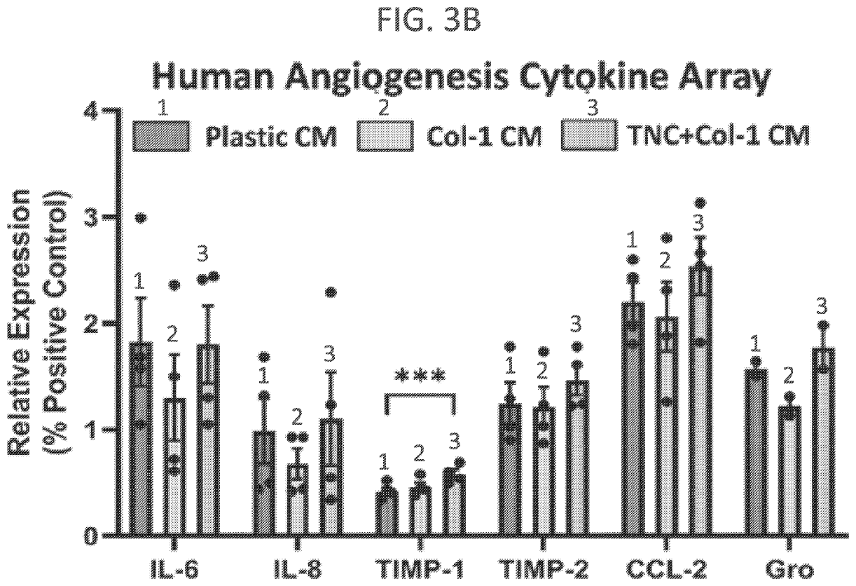

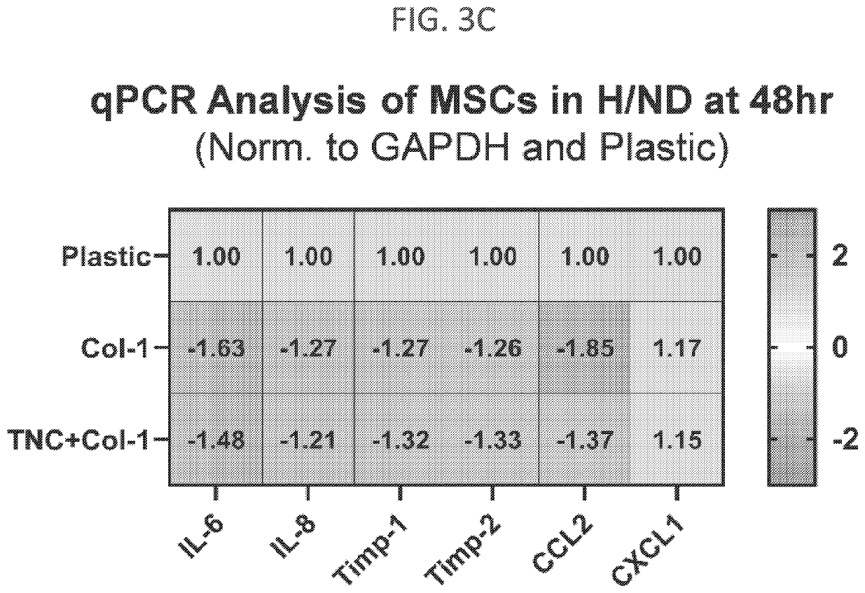

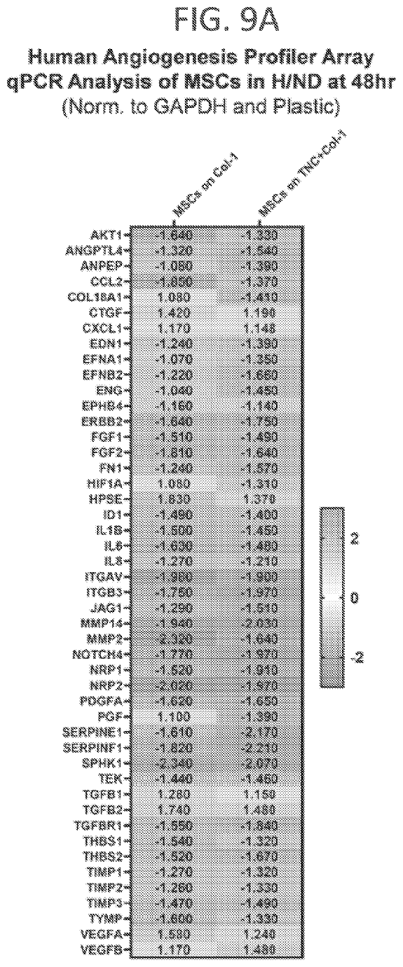

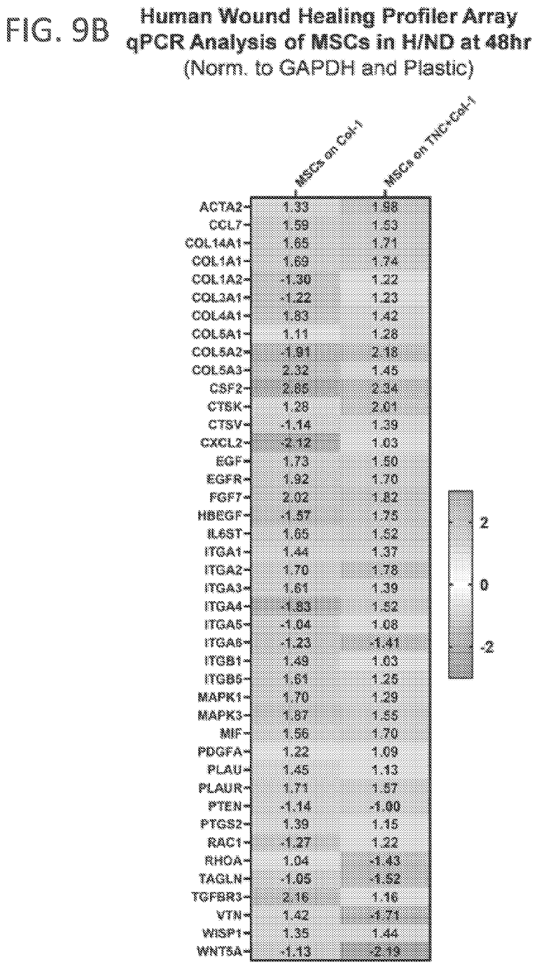

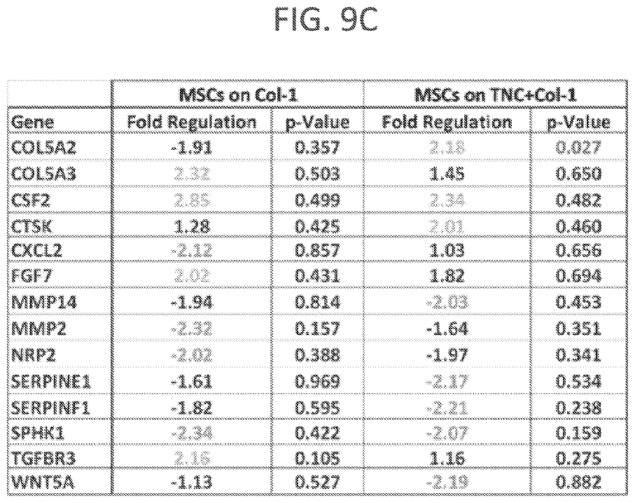

The physiological outcomes of CM on endothelial function prompted exploration of what factors could be at play. Utilizing an aliquot of CM from the different MSC culture conditions, angiogenic cytokines were measured utilizing an angiogenesis array for 43 protein targets (FIGS. 3A, 3B). Of the 43 targets, 6 proteins were substantially expressed including IL-6, IL-8 (CXCL8), TIMP-1, TIMP-2, CCL2 (MCP-1), and Gro alpha/beta/gamma (CXCL1, CXCL2, CXCL3). All protein hits were expressed by all three CM treatment groups, with only TIMP-1 exhibiting a significant difference between CM from plastic and CM from TNC+Col-1, though the fold increase was minimal. Follow-up gene expression analysis for these 6 protein targets revealed no significant differences at the mRNA level between treatment groups (FIG. 3C). To explore other possible differences between the treatment groups, each sample was run on two qPCR profilers to explore gene targets relating to angiogenesis and wound healing (FIGS. 9A, 9B). Out of 138 unique genes in total from both arrays, there were 14 genes that either had an upregulation or downregulation of 2 or more, summarized in the table of FIG. 9C. However, only Col5A2 upregulated in CM from MSC on TNC+Col-1 was statistically significant with a p-value of 0.027.

### Example 5: Coacervate with TNC and MSC Improves Wound Healing In Vivo in Delayed Wound Healing Mouse Model

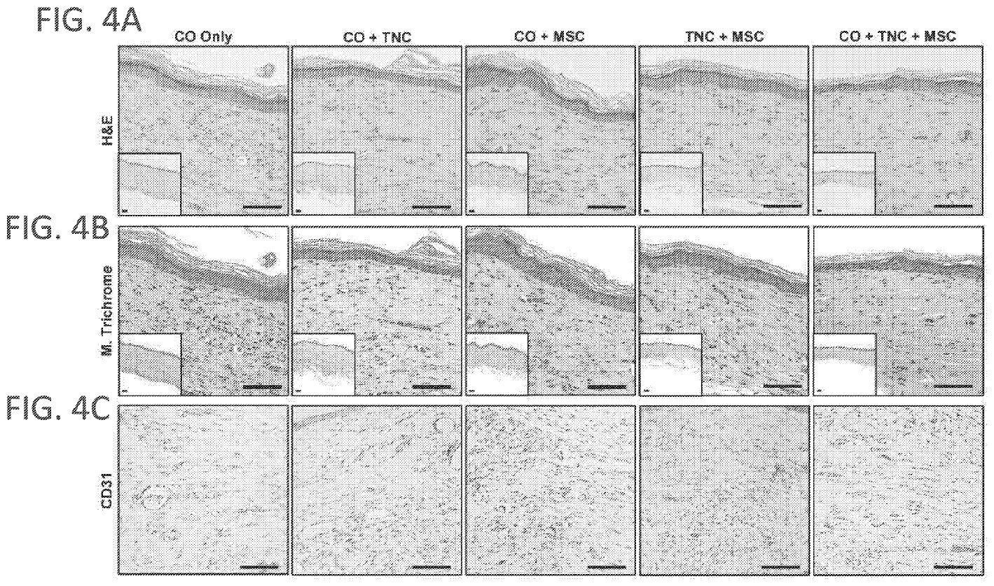

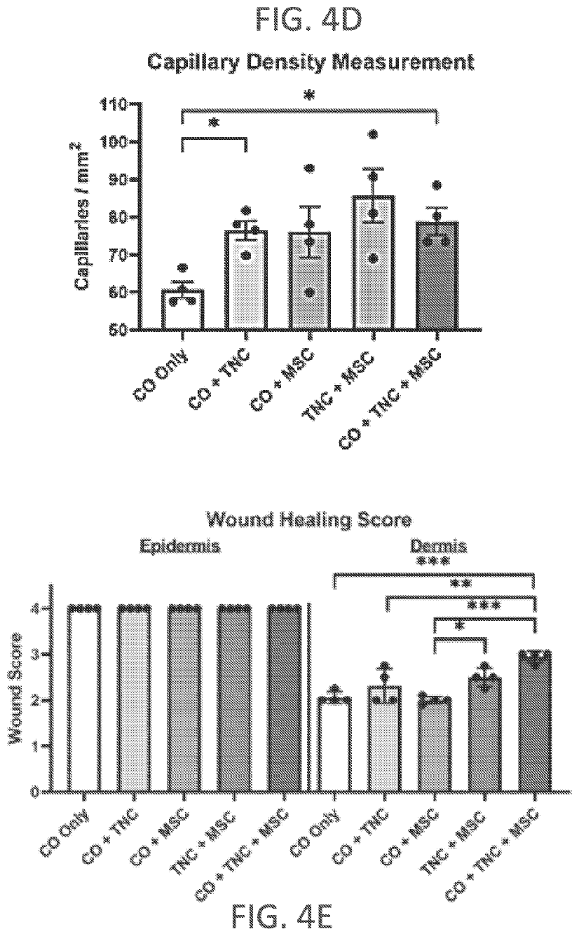

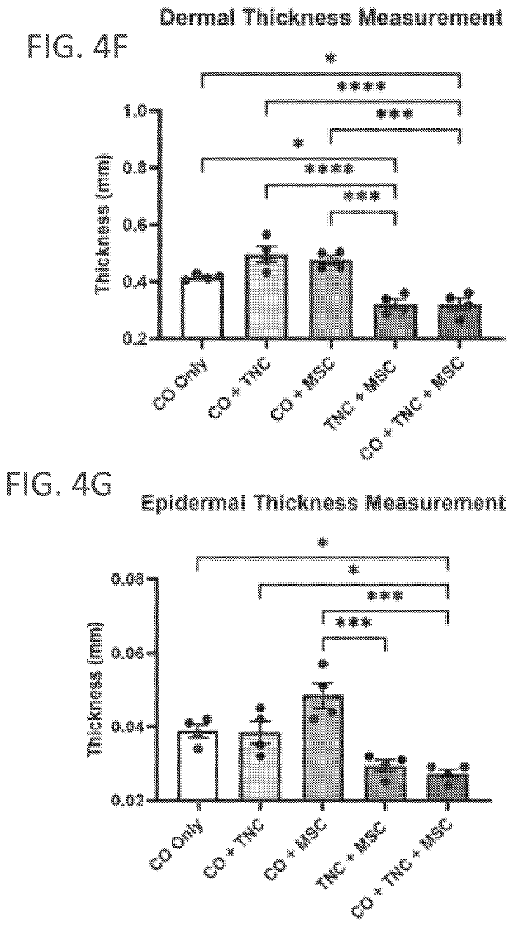

Further studies were performed to determine whether the findings in vitro would improve the healing outcomes in a CXCR3−/− mouse model of delayed wound healing (Yates et al., Wound Repair Regen. January-February 2009; 17 (1): 34-41; Yates et al., Am J Pathol. August 2007; 171 (2): 484-495). Wounds were made in the CXCR3−/− mice, and one of five treatment groups (CO only, CO+TNC, CO+MSC, TNC+MSC, or CO+TNC+MSC) were administered 72 hours after the initial wounding. The wounds were harvested on D30 during the middle of the delayed tissue replacement phase of wound healing (Sylakowski et al., Am J Pathol. July 2020; 190 (7): 1370-1381). At this point in the wound healing process, histological stains using H&E and Mason's Trichrome (MT) reveal complete migration and stratification of the epidermis, along with a fully intact basement membrane indicating complete wound healing of the epidermal layer with a wound score of a 4 (FIGS. 4A, 4B, 4E). However, when looking at epidermal thickness as a restorative measure, treatment with CO+TNC+MSC was significantly thinner than CO Only, CO+TNC, and CO+MSCs. The treatment group with CO+MSCs trended much higher than all the rest in terms of epidermal thickness.

Transitioning to the dermal layer, there was higher cellularity in the mice treated with CO only and CO+MSCs compared to the other treatments (FIGS. 4A, 4B); this indicated the wound healing sequence was delayed with these treatments, and indicated a correction of the defect with the other treatments. Next, staining for platelet endothelial cell adhesion molecule (PECAM-1 or CD31) was performed to assess differences in angiogenesis and capillary formation (FIGS. 4C, 4D). A statistically significant higher number of capillaries were observed in mice treated with CO+TNC+MSCs and CO+TNC compared to CO only control. When examining the MT staining, the CO+TNC treatment group appeared to have the highest amount of granulation collagen due to the lightness of the blue color (FIG. 4B). The mice treated with CO+TNC+MSCs exhibited the darkest color of collagen, signifying higher collagen turnover from immature Col-III to mature Col-I.

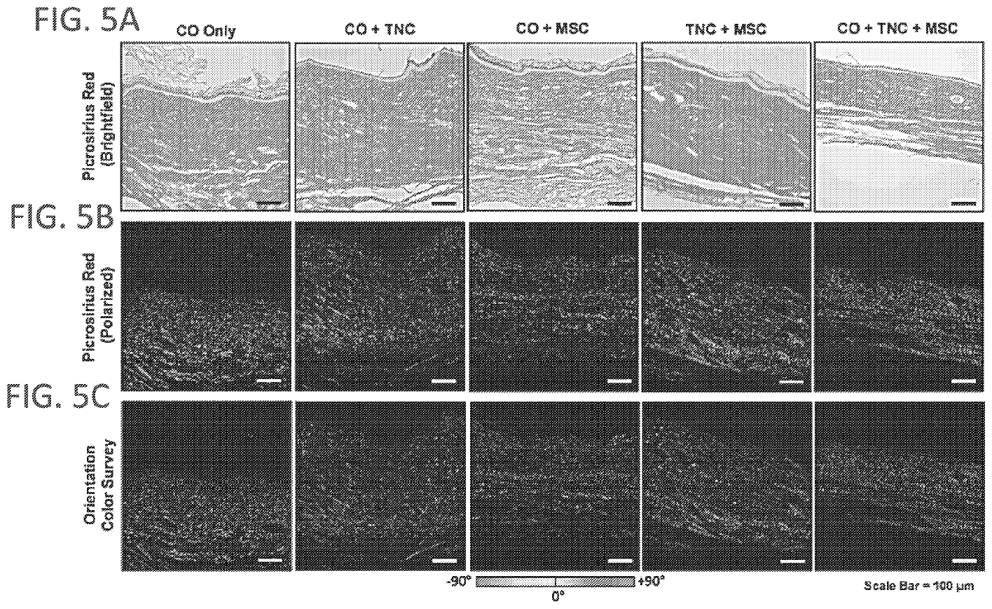

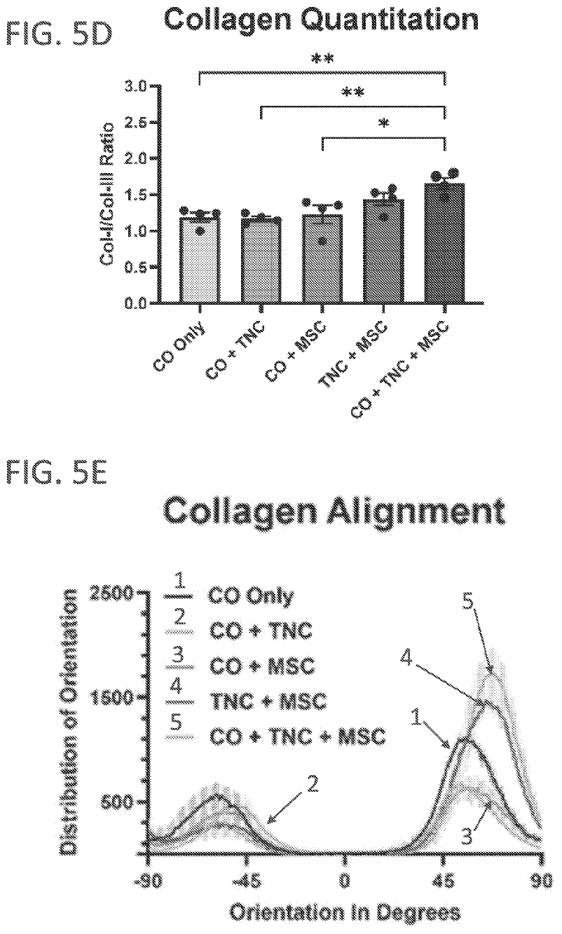

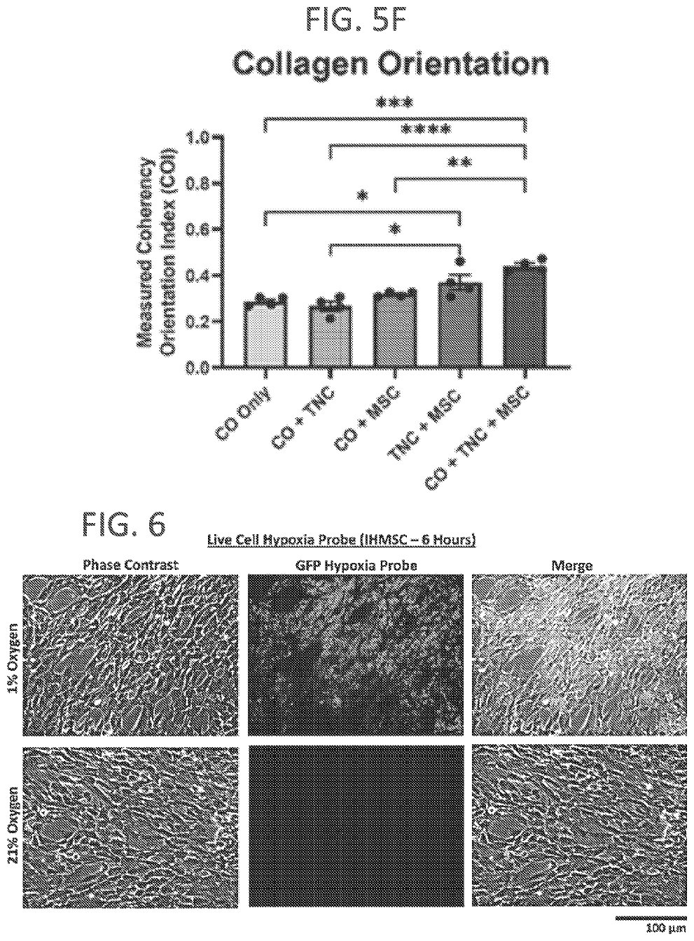

Further quantification of collagen Col-1/Col-III ratio using Picrosirius Red (PSR) stain confirmed this observation, where mice treated with CO+TNC+MSC had a significantly higher Col-1/Col-III ratio compared to CO only, CO+TNC, and CO+MSC (FIGS. 5A, 5B, 5D). The CO only, CO+TNC, and CO+MSC treatment groups had a Col-1/Col-III ratio close to 1, indicating they were still early in the tissue replacement phase with a lot of granulation tissue. The CO+TNC+MSC mice had a Col-1/Col-III ratio above 1.5 and approaching 2, indicating active collagen maturation and entry into the resolution phase of wound healing. Normal unwounded skin has a Col-1/Col-III ratio of 2 or higher.

In addition to collagen content quantification, collagen alignment scoring can also help determine the maturity of collagen within the dermis. Upon healing a wound within the murine model who doesn't have rete ridges, the dermal layer alignment becomes more horizontal as the collagen becomes more mature and contracted via the myofibroblasts. The overall alignment of the collagen fibrils was observed using the ImageJ color survey tool (FIG. 5C). This allows the PSR stain to be pseudo-colored according to their alignment orientation. From this view, the orientation can be analyzed in two ways. The first collagen alignment method was to look at the total distribution of all collagen fibrils relative to their degree of alignment (FIG. 5E). A skewed bimodal distribution was seen in all treatment groups with one peak around the −50 degree orientation mark and the other around the +50 degree orientation mark. The mice treated with the CO+TNC+MSC group had the tallest peak out of all the treatment groups at the +50 mark, indicating it has the most alignment in that location. It is also the highest peak out of all peaks on the graph, signaling it is the most aligned of all treatment groups. The two treatment groups with the lowest collagen alignment score were CO+TNC and CO+MSCs. The second way to measure collagen alignment is through total measured coherency. This measures the overall coherency of the collagen fibrils within each image with a total coherency measure between 0 and 1, where 0 indicates complete isotropy and 1 indicates complete alignment (Rezakhaniha et al., Biomech Model Mechanobiol. March 2012; 11 (3-4): 461-473). Similar to what was observed in the first collagen alignment method, the mice treated with CO+TNC+MSCs had a significantly higher coherency score compared to CO only, CO+TNC, or CO+MSCs (FIG. 5F). The mice treated with TNC+MSC without coacervate also exhibited good alignment with coherency scores significantly higher than CO only and CO+TNC treatment groups. For one last measure of collagen maturation in terms of contraction, mice treated with CO+TNC+MSCs and TNC+MSCs presented significantly thinner dermal thickness scores than the other three treatment methods (FIG. 4F). Overall, mice treated with CO+TNC+MSCs had the highest wound score, closely followed by TNC+MSCs without coacervate (FIG. 4E), whereas mice treated with CO only and Co+MSCs exhibited the least amount of maturation in most metrics. These findings demonstrate that TNC inclusion improves the performance of MSCs in promoting maturation of wounds through the phases of healing.

### Example 6: Background, Materials and Methods

Cutaneous wound healing is a highly complex and dynamic process that requires a successful transition through three phases of repair to reach a successful resolution. This process relies critically on the timing and balance of key cell types, growth factors, and extracellular matrix (ECM) molecules to maintain proper outcomes. However, patients who have experienced extreme trauma or severe burns can disrupt this complex network of pro-fibrotic and anti-fibrotic interactions and signals, leading to an excessive deposition of collagen and the formation of hypertrophic scars (HTS) (Zhu et al., Burns Trauma. 2016; 4:2). Patients with severe HTS are subject to high psychological and physical impairment levels, as these wounds are often found in areas of high skin tension and can be very resistant to treatment or therapy (Bock et al., Arch Dermatol Res. April 2006; 297 (10): 433-438).

One of the critical elements of HTS is the imbalance of the ECM throughout the wound healing process. Early in the HTS wound healing process, the wound micro-environment is often plagued with chronic inflammation leading to an overabundance of matrix metalloproteinases (MMPs) and disproportionate breakdown of ECM (Zhu et al., Burns Trauma. 2016; 4:2; Yates et al., Birth Defects Res C Embryo Today. December 2012; 96 (4): 325-333; Ghazawi et al., Adv Skin Wound Care. January 2018; 31 (1): 582-595; Gauglitz et al., Mol Med. January-February 2011; 17 (1-2): 113-125). This in turn, creates a response for fibroblasts in the next phase of repair to excessively proliferate and produce an overabundant amount of collagen, ultimately leading to HTS (Zhu et al., Burns Trauma. 2016; 4:2; Yates et al., Birth Defects Res C Embryo Today. December 2012; 96 (4): 325-333; Ghazawi et al., Adv Skin Wound Care. January 2018; 31 (1): 582-595; Gauglitz et al., Mol Med. January-February 2011; 17 (1-2): 113-125; Oliveira et al., Int Wound J. December 2009; 6 (6): 445-452). In addition to the changes observed to ECM structural proteins such as collagen in HTS patients, other non-structural ECM proteins called matricellular proteins are also affected. These specialized ECM proteins do not provide structure or scaffolding support for cells; instead, they directly interact with cell signaling through the interaction of growth factors, growth factor receptors, and other ECM proteins within the wound microenvironment, driving a variety of biological signaling cascades. One of these vital matricellular proteins is called decorin, and it acts as a stop signal within the resolution phase of wound healing.

Decorin (DCN) is a small leucine-rich proteoglycan roughly 100 kDa in size. It consists of a core protein element composed of 10-12 tandem leucine-rich repeats (LRR) attached to a single N-terminal glycosaminoglycan chain (GAG) (Chen and Birk, Front Pharmacol. 2019; 10:1649; Pang et al., Front Pharmacol. 2019; 10:1649). Its name is derived by its ability to bind or “decorate” collagen fibrils and is found throughout the reticular dermis of the skin (Pang et al., Front Pharmacol. 2019; 10:1649). DCN's ability to interact with collagen allows for it to be a key component of collagen fibrillogenesis during wound healing as it helps to regulate collagen fibril diameter, organization, and spacing between collagen strands (Kadler et al., Curr Opin Cell Biol. October 2008; 20 (5): 495-501; Danielson et al., J Cell Biol. Feb. 10, 1997; 136 (3): 729-743). Additionally, it can regulate other signaling cascades within the wound microenvironment, often acting as a non-competitive antagonist for a host of growth factors and growth factor receptors (Buraschi et al., Matrix Biol. January 2019; 75-76:260-270; Merline et al., J Cell Commun Signal. December 2009; 3 (3-4): 323-335; Neill et al., Am J Pathol. August 2012; 181 (2): 380-387; Gubbiotti et al., Matrix Biol. September 2016; 55:7-21). One critical signaling receptor DCN will bind to during the resolution phase is the vascular endothelial growth factor (VEGF) receptor 2 of endothelial cells, inducing an autophagic response and suppressing angiogenesis during this repair phase (Sylakowski and Wells, Matrix Biol. June 2021; 100-101:197-206; Buraschi et al., Proc Natl Acad Sci USA. Jul. 9, 2013; 110 (28): E2582-2591). DCN also regulates transforming growth factor-beta-1 (TGF-β1), a significant influencer of wound contraction, by either binding and neutralizing the growth factor directly or by downregulating its production from hypertrophic scar fibroblasts (Gubbiotti et al., Matrix Biol. September 2016; 55:7-21; Zhang et al., Burns. August 2007; 33 (5): 634-641; Zhang et al., Burns. June 2009; 35 (4): 527-537).

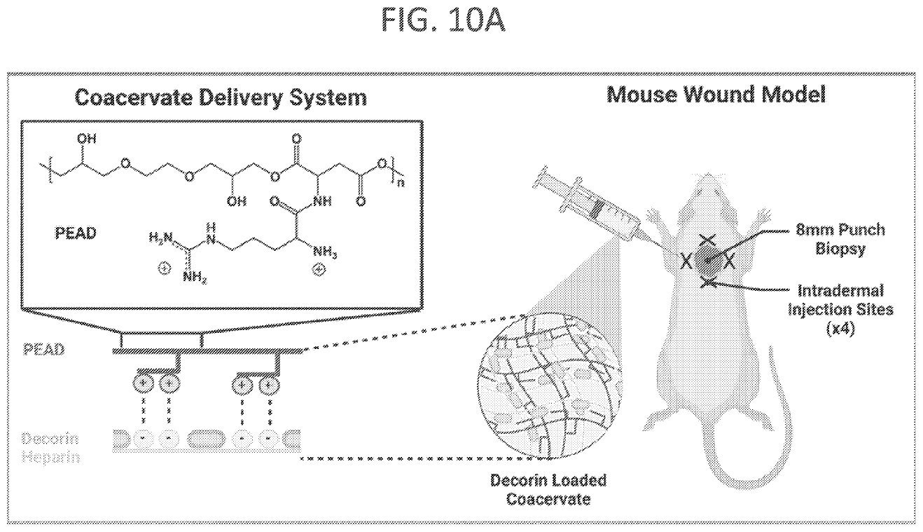

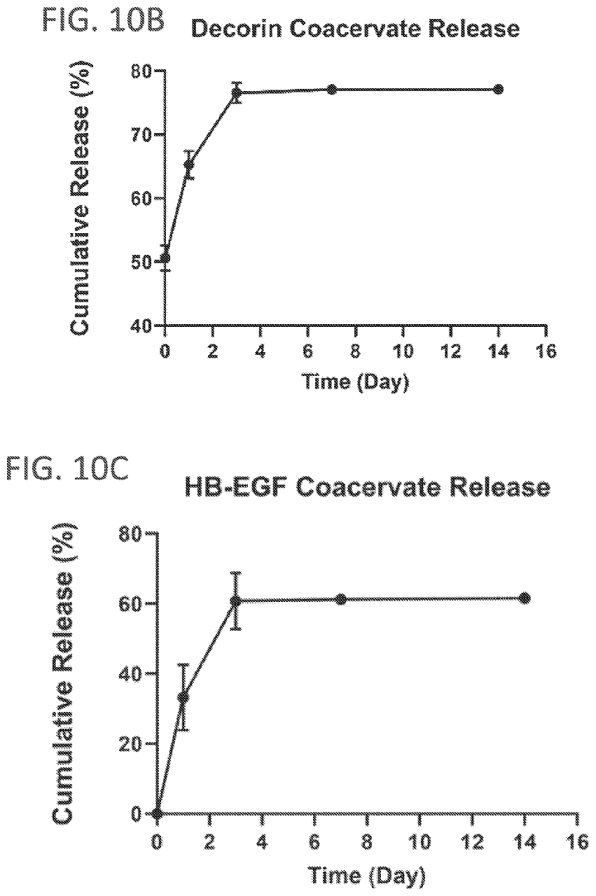

CXCR3 is a seven transmembrane G-protein coupled receptor that is the sole receptor for CXCL4, CXCL9, CXCL10 and CXCL11, the latter two of which are expressed during wound healing at the time of the transition from tissue replacement to wound resolution. Mice lacking the CXCR3 receptor display a delayed but prolonged dermal wound healing phenotype that leads to hypertrophic scarring (Yates et al., Am J Pathol. August 2007; 171 (2): 484-495; Yates et al., Am J Pathol. April 2010; 176 (4): 1743-1755). Correction of the scarring defect using cellular transplants reverses the matrix progression back towards near regenerative healing (Yates et al., Stem Cell Res Ther. Sep. 5, 2017; 8 (1): 193; Yates et al., Cell Transplant. Jan. 24, 2017; 26 (1): 103-113; Sylakowski et al., Am J Pathol. July 2020; 190 (7): 1370-1381). Thus, studies described below were performed to determine if directly pharmacologically altering the matrix can achieve better healing as it would be easier than cellular transplants. DCN plays an important role in proper wound resolution, and its protein levels are found to be decreased roughly 75% in HTS compared to regular wound healing patients (Gauglitz et al., Mol Med. January-February 2011; 17 (1-2): 113-125; Finnerty et al., Lancet. Oct. 1, 2016; 388 (10052): 1427-1436). However, delivery of exogenous growth factors or other protein-based therapies is often limited in their efficacy due to extensive degradation once administered in vivo into the harsh wound microenvironment (Hwang et al., Front Bioeng Biotechnol. 2020; 8:69). To overcome this challenge, DCN was encapsulated in a heparin-mediated coacervate system. This system is an injectable in vivo delivery vehicle that uses a positively charged synthetic biodegradable poly(ethylene arginyl aspartate diglyceride) (PEAD) and a negatively charged heparin to form a 3-dimensional coacervate (Johnson and Wang, Wound Repair Regen. July-August 2015; 23 (4): 591-600; Park et al., Acta Biomater. May 2019; 90:179-191; Chu et al., J Control Release. Mar. 10, 2011; 150 (2): 157-163; Johnson and Wang, J Control Release. Mar. 10, 2013; 166 (2): 124-129). This delivery approach allows for the protection of the DCN while also allowing for its controlled release over several days (FIG. 10A). As a control modulator, heparin-binding EGF-like growth factor (HB-EGF) was used as it has been shown to accelerate delayed healing in a mouse model of diabetic wounds (Johnson and Wang, Wound Repair Regen. July-August 2015; 23 (4): 591-600). It was hypothesized that delivery of DCN would result in a reduced scarring phenotype and an overall better healing outcome in the hypertrophic scarring mouse model.

### Animal Model

C57BL/6J CXCR3−/− mice were generated as previously described (Hancock et al., J Exp Med. Nov. 20, 2000; 192 (10): 1515-1520). The C57BL/6J CXCR3−/− mice were then bred to FVB strain mice for at least 20 generations to yield germline transmission of the targeted allele and create CXCR3 devoid mice on the FVB background. For this study, CXCR3/female mice were bred with CXCR3/males and all offspring were screened for genotype through PCR before use. Serological analyses did not detect blood-borne pathogens or evidence of infection. Mice were housed in individual cages after wounding and maintained under a 12-hour light/dark cycle.

### Wounding

Male and female mice (6 to 8 months of age and weighing approximately 25 g) were anesthetized with an intraperitoneal injection containing ketamine (75 mg/kg) and xylazine (5 mg/kg). The backs were cleaned, shaved, and sterilized with betadine solution. Full-thickness wounds were performed using an 8 mm diameter punch biopsy through the epidermal and dermal layers along one side of the dorsal midline of the mouse. The contralateral uninjured skin served as unwounded control skin.

### Coacervate Preparation

PEAD was synthesized as previously described (Chu et al., J Control Release. Mar. 10, 2011; 150 (2): 157-163). Stock solutions of PEAD and heparin (10 mg/mL) were prepared in 0.9% saline solution and passed through a 0.22 μm sterile syringe filter. Recombinant human HB-EGF (Cat. No. 50-398-861, PeproTech) and DCN (Cat. No. 143DE100 R&D Systems) were each reconstituted to 300 μg/mL and mixed with heparin stock solution at their respective predetermined working doses of 1.45 μg for HB-EGF (Johnson and Wang, Wound Repair Regen. July-August 2015; 23 (4): 591-600) and 10 μg for DCN (Krishna et al., Otolaryngol Head Neck Surg. December 2006; 135 (6): 937-945). The heparin/HB-EGF and heparin/decorin solutions were then each mixed with PEAD stock solutions to form protein-encapsulated coacervates at a final ratio of 1:100:500 (protein: heparin: PEAD) mass ratio. Coacervation proceeded rapidly with the formation of a turbid solution once all components were mixed.

### Coacervate Mouse Injections

Intradermal injections were administered once on day 7 for mice being harvested on day 14. Whereas mice that were harvested at day 30 or 90 received two rounds of injections, at day 7 and day 14. For each treatment, a mouse received a total of 200 μL of either treatment coacervate or control coacervate through a series of 4 injections of 50 μL around the wound bed to avoid tissue trauma (FIG. 10A). Mice treated with HB-EGF coacervate received a dose of 200 μL containing a total of 1.45 μg HB-EGF, while mice treated with DCN received a dose of 200 μL containing a total of 10 μg DCN.

### Coacervate Release

Coacervate was prepared as stated above to create three independent samples for both HB-EGF and DCN. Samples were incubated at 37° C. for 14 days, and the supernatant was harvested on days 0, 1, 3, 7, and 14 through pelleting the coacervates by centrifugation (12,100 g for 10 min). Supernatants were analyzed using pre-coated enzyme-linked immunosorbent assay (ELISA) kits (Cat. No. EHHBEGF and Cat. No. EHDCN, Thermo Fisher Scientific), according to the manufacturer's specifications. The release profiles were as previously published for HB-EGF (FIG. 10C) (Johnson and Wang, J Control Release. Mar. 10, 2013; 166 (2): 124-129), and slightly faster for DCN as befits its larger size and lack of proteoglycan binding domain (FIG. 10B).

### Histological Analyses

Wound bed biopsies were collected after euthanasia at days 14, 30, and 90 using a 12 mm punch biopsy and were immediately fixed in 10% buffered formalin. Samples were then paraffin embedded (FFPE), sectioned into slides (6 μm), and stained with hematoxylin and eosin (H&E) and Masson's Trichrome (MT). Blank slides were also made for downstream Picrosirius Red (PSR) staining as directed by the manufacturer's protocol (Cat. No. 150681, Abcam) and followed by immunohistochemistry and immunofluorescent stains.

Wound healing score assessment—Histopathological examination of mouse tissues was performed in a double-blind approach among three investigators, and their scores averaged. Qualitative assessments were based on epidermal and dermal maturation as outlined in FIG. 14 and previously established protocols (Yates et al., Am J Pathol. August 2007; 171 (2): 484-495; Yates et al., Wound Repair Regen. January-February 2009; 17 (1): 34-41; Yates et al., Am J Pathol. September 2008; 173 (3): 643-652). In short, the samples were scored on a scale of 0 to 4 for both epidermal and dermal layers of tissue. For epidermal maturation scoring: 0=no epidermal migration, 1=partial epidermal migration, 2=complete epidermal migration, 3=partial keratinization and an intact basement membrane, 4=complete keratinization and normal epidermis. For dermal maturation scoring: 0=no healing, 1=inflammatory infiltration, 2=granulation tissue present-fibroplasias and angiogenesis, 3=collagen deposition replacing granulation tissue>50%, 4=complete replacement of granulation tissue and complete healing.

Skin thickness measurements—H&E and MT sample images were analyzed using ImageJ line measurement tool to measure the thickness of the epidermal layer and dermal layer for each mouse specimen. The epidermal layers were measured from the stratum basale to the stratum granulosum (excluding the stratum corneum); and the dermal layer was measured form the top of the papillary layers to the bottom of the reticular layer. A total of 6 measurements per biological sample were scored for each layer.

Collagen quantification—Polarized PSR images were quantified using ImageJ version 1.53K. Each image was split into red, green, and blue channels, from which the red and green channels were selected. The isolated red and green channels then underwent thresholding and follow-up measurements to determine the total staining area for each collagen type. PSR birefringence under polarized light reveals tightly packed thick and long fibrils of type 1 collagen as either bright red-orange intense birefringence in tissue and thin short loose fibrils as yellow-green (Coelho et al., An Bras Dermatol. June 2018; 93 (3): 415-418). The overall ratio of type 1 collagen to type III collagen was analyzed by comparing the representative staining results for either collagen type back to the original image for total collagen content.

Collagen orientation and alignment—Polarized PSR images were quantified using ImageJ plug-in: OrientationJ version 2.0.5. Visual directional analysis using the color survey tool was performed with the following parameters: Hue was set for orientation, Saturation was set for coherency, and brightness was set to the original image (Puspoki et al., Adv Anat Embryol Cell Biol. 2016; 219:69-93). The OrientationJ distribution tool was used to create the distribution of orientation histograms to determine where patterns of alignment were within samples (Rezakhaniha et al., Biomech Model Mechanobiol. March 2012; 11 (3-4): 461-473). The OrientationJ Dominant Direction tool was used to determine the quantitative orientation measurement for coherency across the whole image (Fonck et al., Stroke. July 2009; 40 (7): 2552-2556).

Immunohistochemistry staining—Skin sample tissue sections (6 μm) were rehydrated and placed into citrate acid buffer at 95° C. for 10 minutes and allowed to cool to room temperature for 1 hour. Samples were then washed in phosphate-buffered saline (PBS) and quenched with 3% hydrogen peroxide. Following additional washes in PBS, samples were blocked in 20% goat serum for 1 hour and subsequently incubated with primary antibodies diluted in blocking buffer overnight at 4° C. Primary antibodies were used at the following dilutions: CD45 (1:100, Cat. No. ab10558, Abcam) and Col-IV (1:500, Cat. No. ab236640, Abcam). After primary antibody incubation, samples were washed in PBS and incubated in goat anti-rabbit HRP secondary antibody (1:2000, Cat. No. ab205718, Abcam) for 1 hour at room temperature. After final antibody incubations, samples were washed in PBS and then treated with ABC reagent (Cat. No. PK-6100, Vector Laboratories) for 30 minutes. Samples were then rewashed with PBS and developed with DAB reagent kit 27 (Cat. No. SK-4100, Vector laboratories), counterstained with H&E, clarified (Richard Alan Scientific), and dehydrated in ethanol and xylene. Samples were mounted (Cat. No. SP15-500, Fisher Scientific) and imaged using an Olympus Provis microscope. Image analysis was performed using Image J Colour Deconvolution 2 plug-in to acquire DAB positive staining area. The total DAB positive area was compared back to the total area of the original image to get a final percentage.

Immunofluorescence staining—Skin sample tissue sections (6 μm) were rehydrated and placed into citrate acid buffer at 95° C. for 10 minutes and allowed to cool to room temperature for 1 hour. Samples were then washed in PBS and quenched with 3% hydrogen peroxide. Following additional washes in PBS, samples were blocked in 20% goat serum for 1 hour and subsequently incubated with primary antibodies diluted in blocking buffer overnight at 4° C. Primary antibodies were used at the following dilutions: CD31 (1:50, Cat. No. ab28364, Abcam) and Involucrin (1:500, Cat. No. 924401, Biolegend). After primary antibody incubation, samples were washed in PBS and incubated in goat anti-rabbit 488 secondary antibody (1:200, Cat. No. ab150077, Abcam) for 1 hour at room temperature in the dark. Samples were washed in PBS and mounted in Prolong Gold Antifade Mountant with DAPI (Cat. No. P36931, Invitrogen). Fluorescent images were obtained using an Olympus Provis microscope.

### Statistical Analysis

Results for wound healing score assessment and PSR staining assessment were expressed as mean±SD with at least a minimum of four mice per treatment group. Histological quantifications for skin layer thickness and immunohistological stains (CD31, CD45, Col-IV) were performed on five microscopic fields of each specimen and reported as mean±SEM. Statistical differences between groups were determined by one-way ANOVA, followed by post-hoc Tukey HSD analysis using GraphPad Prism 9 software (GraphPad Software, San Diego, CA). Significance was claimed for P<0.05.

### Example 7: Growth Factor Therapy Augments Initial Phases of Healing

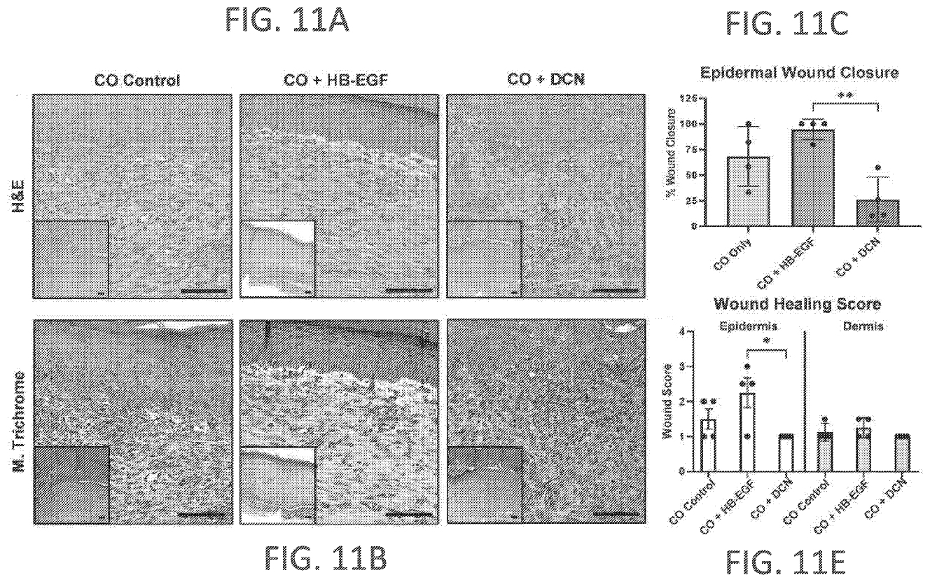

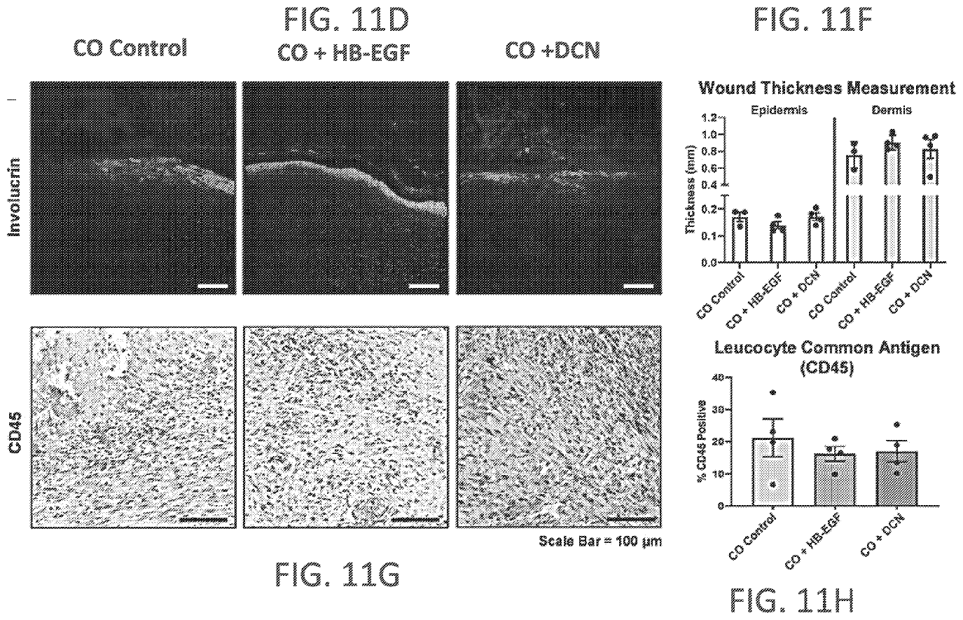

Wounds made in the CXCR3−/− mice were treated 7 days after initial wounding using DCN or HB-EGF encapsulated in coacervate. These mice were then analyzed on day 14, during the later stages of the inflammation phase of wound healing (Wells et al., Matrix Biol. January 2016; 49:25-36). Mice treated with CO+HB-EGF has increased epidermal closure compared to CO+DCN or CO only treated mice (FIG. 11A-11C). CO+HB-EGF treated mice exhibited complete epithelial cell migration in three of four mice, allowing for the early elimination of the eschar. An additional marker for determining epidermal maturation was used by examining a marker for keratinocyte cohesion, involucrin. In an early, less mature epidermis, the involucrin protein is localized in the cytoplasm of keratinocytes as exhibited in CO only, and CO+DCN treated mice (FIG. 11D). However, in a more mature epidermis, keratinocytes have secreted the involucrin outside the cell to help form a protective boundary and allow the complete formation of the keratinized stratified epithelium as exhibited by the CO+HB-EGF mice (FIG. 11D) (Eckert et al., J Invest Dermatol. May 1993; 100 (5): 613-617). These results demonstrated that CO+HB-EGF treated mice had significantly faster epidermal wound healing than CO+DCN treated mice (FIG. 11E).

Upon examining the dermal layer of tissue, histological stains H&E and MT revealed a high amount of cell infiltrate within each treatment group (FIGS. 11A, 11B). Staining for leucocyte common antigen (CD45) was used to assess differences in immune cell infiltrate (FIG. 11G). However, no significant differences were found among treatment groups after quantification (FIG. 11H). The thickness of the dermis was also measured (FIG. 11F) but no significance was found. These findings showed no significant difference between treatment groups regarding the overall dermal wound healing score (FIG. 11E). These findings indicate that the epidermal closure can be accelerated by upregulating the growth factor signaling early in wound healing. However, the dermal healing proceeds while introducing DCN, a healing ‘stop’ signaling, does not affect the inflammatory phases of wound healing.

### Example 8: Modifying the Matrix Increased Vascularization During Tissue Replacement

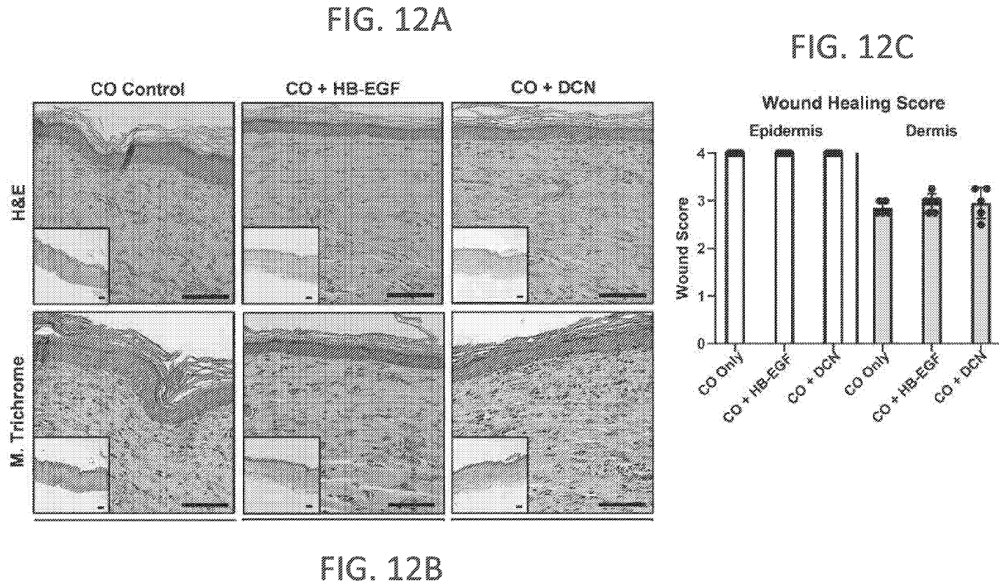

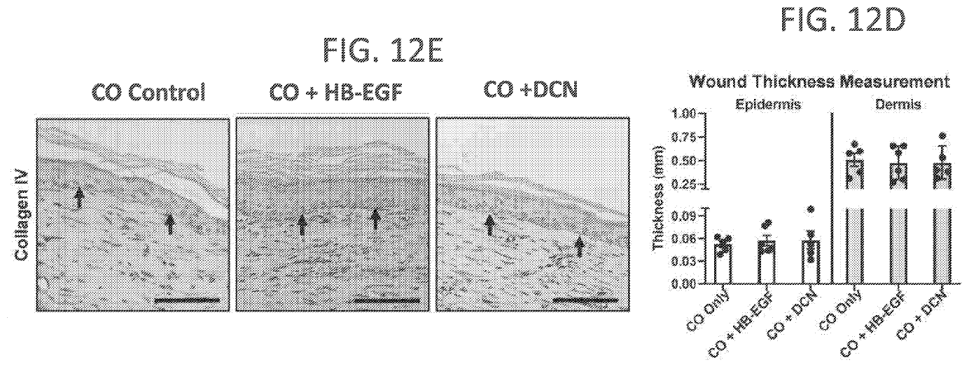

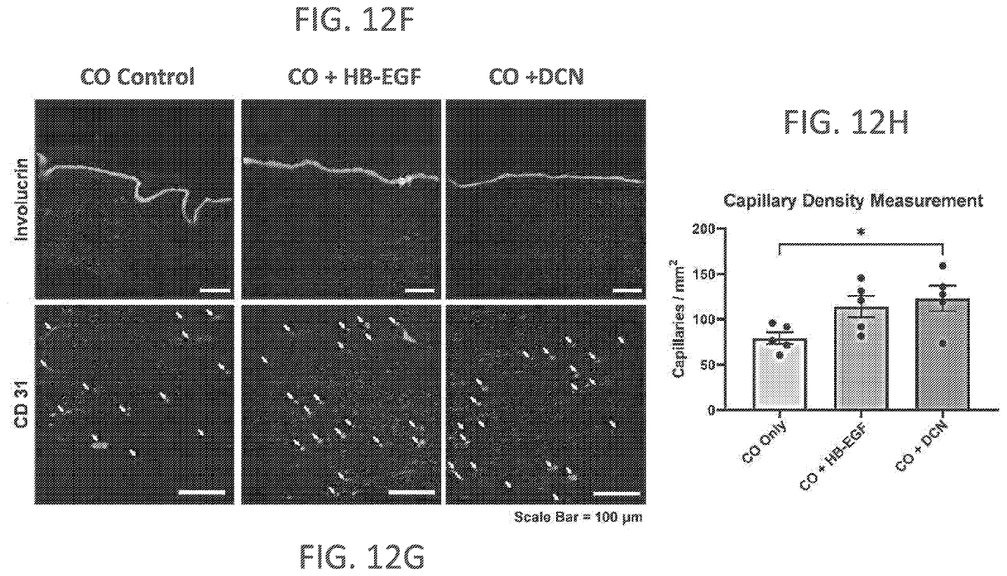

Continuing onto the findings of delayed dermal maturation of CXCR−/− mice, it was observed whether any coacervate treatment group helped to improve outcomes during the tissue replacement phase of repair by examining outcomes at 30 days. At this point in the wound healing process, histological stains using H&E and MT reveal complete migration and stratification of the epidermal layer, including eliminating the eschar in all three treatment groups (FIGS. 12A, 12B). Additional staining with involucrin showed a complete protective barrier signifying the completion of the stratified corneum in the epidermis in all three treatment groups (FIG. 12F). The final stain of Col-IV revealing the formation of an intact basement membrane fully separating the epidermis from the dermis (FIG. 12E), demonstrates that the epidermal layer has reached a total wound healing score of 4 in all three treatment groups (FIG. 12C).

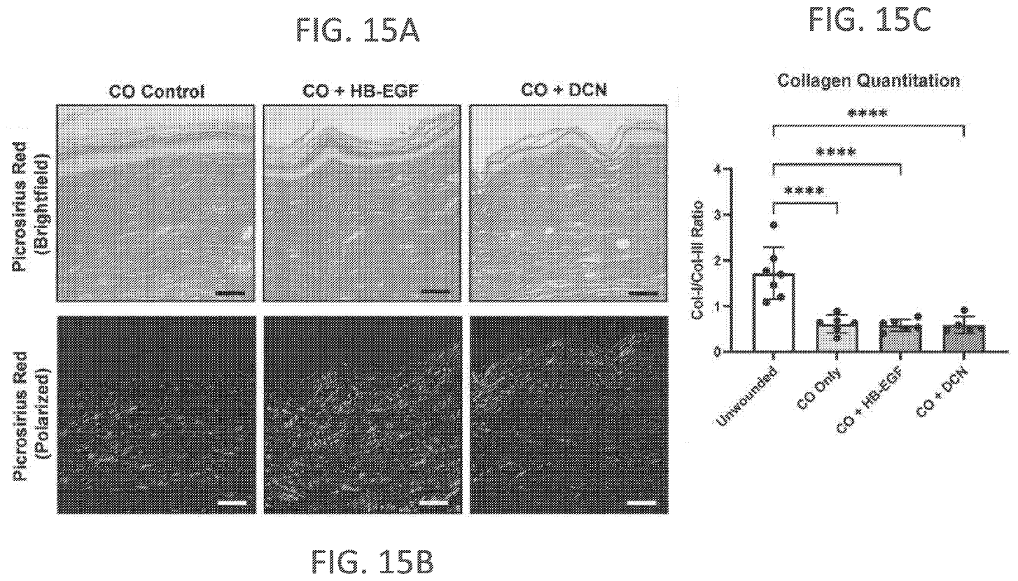

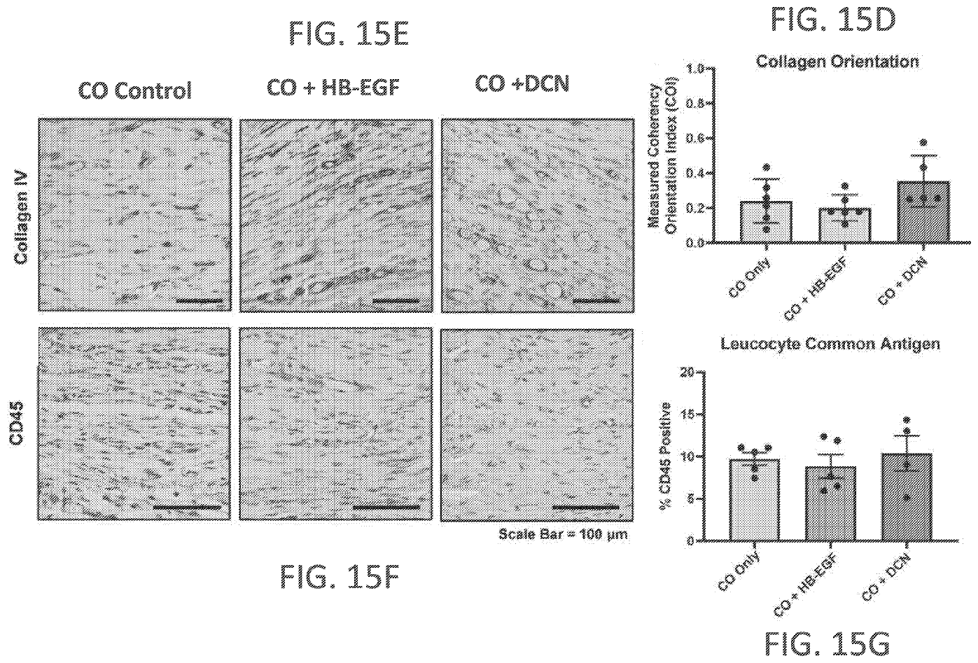

Further histological examination of the dermal tissue layer did not reveal a significant difference, with all treatment groups averaging a wound healing score of 3 and the exact thickness measurement of roughly 0.50 mm (FIGS. 12C, 12D). In the MT staining, the CO only mice appeared to have slightly less collagen than the other two treatment groups (FIG. 12B). However, further quantification of collagen Col-1/Col-III ratio and alignment using PSR stains revealed no significant differences (FIGS. 15A-15D). Histological examination also revealed an overall decrease in cellularity compared to D14 samples (FIGS. 12A, 12B). The presence of immune cell infiltrate with CD45 was assessed but a significant difference was not observed among treatment groups (FIGS. 15F, 15G). However, surprisingly, there was a significant increase in capillary formation of CO+DCN treated mice compared to CO only treated mice confirmed through CD31 and Col-IV staining (FIGS. 12G, 12H, 15E). However, even with increased vascularity, this did not change the overall wound scoring during this phase of repair.

### Example 9: Coacervate with Decorin Improves Wound Healing Outcomes in the Resolution Phase

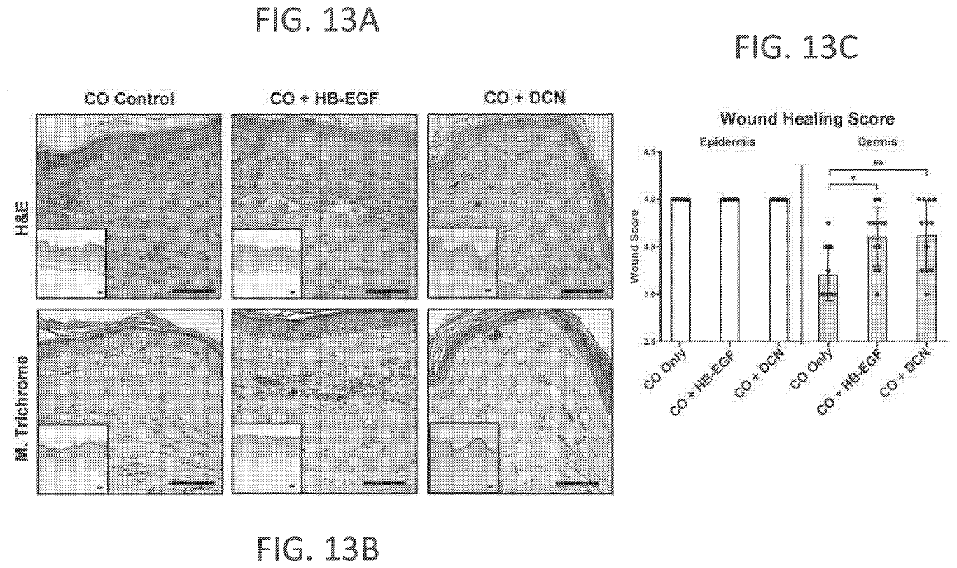

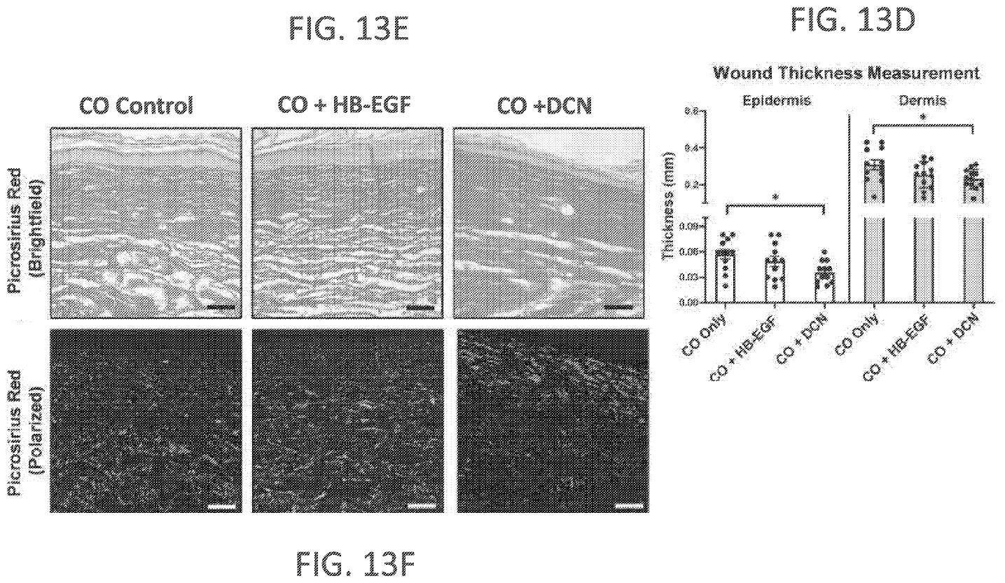

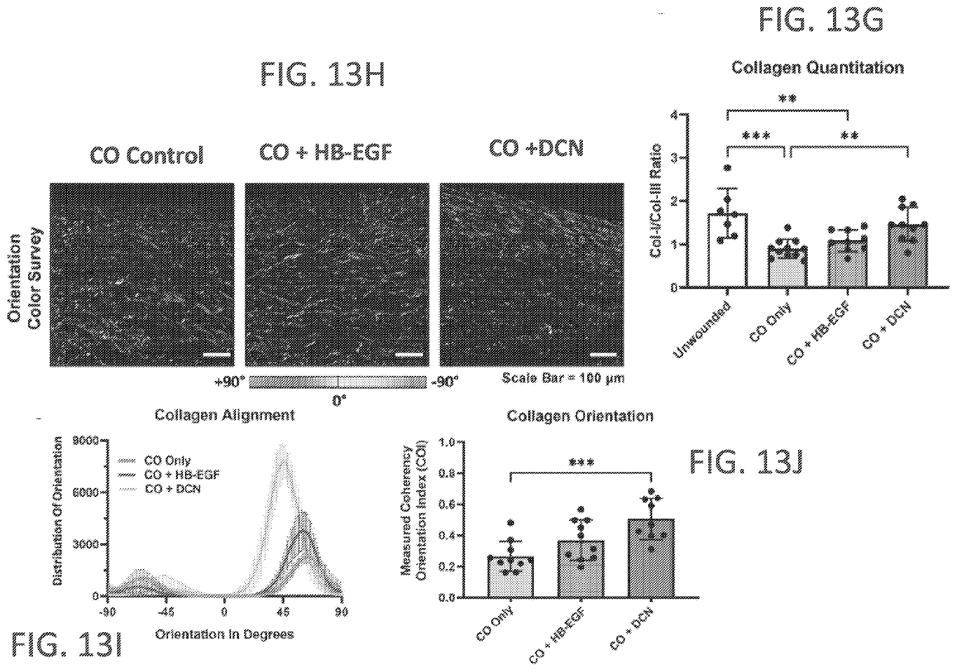

CXCR3 knockout mice exhibit hypertrophic scarring by day 90 after the initial wound (Yates et al., Am J Pathol. April 2010; 176 (4): 1743-1755); this was predicted to be the time point during wound resolution that ‘stop’ signals would exert their most significant effects. To this point, significantly improved wound healing scores were observed in mice treated with CO+DCN as compared to CO only mice (FIGS. 13A-13C). CO+DCN treated mice exhibited less overall cellularity within the dermal layer of tissue in addition to more organized collagen fibrils. Additionally, CO+DCN treated mice presented a significantly thinner epidermal and dermal wound thickness compared to CO only mice as these resolved back towards unwounded thicknesses (FIG. 13D). CO+HB-EGF also exhibited a significantly higher dermal wound score over CO only mice but did not significantly change the overall thickness measurement for either the epidermal or dermal layers, suggesting that this treatment did not drive resolution.

The collagen content and alignment of PSR staining was further assessed (FIG. 13E). Using polarized light microscopy on PSR stained samples, the natural birefringence of collagen fibers allows for the evaluation of collagen organization and separation into either collagen type I displaying a red-orange color or collagen type III with a green-yellow color (FIG. 13F) (Coelho et al., An Bras Dermatol. June 2018; 93 (3): 415-418). Upon quantifying collagen content, it was observed that the mice treated with CO+DCN had a significantly higher ratio of Col-1/Col-III compared to that of CO only treated mice, returning the collagen content ratio to that observed in unwounded skin samples (FIG. 13G). Conversely, both CO only and HB-EGF treated mice had significantly lower ratios of Col-1/Col-III, suggesting these mouse populations are still in the process of turning over the remaining granulation tissue from the tissue replacement phase.

The orientation of the collagen in the PSR stains was assessed next. Utilizing the ImageJ plug-in OrientationJ, the overall profile alignment of collagen was measured for an entire specimen as well as the overall coherency. First, the overall alignment of the collagen fibrils was observed using the color survey analysis tool, which allows pseudo coloring of the PSR images to hues that represent certain degrees of alignment (FIG. 13H). This was further quantified in FIG. 13I, to show the total distribution of all collagen fibrils relative to their degree of alignment. All treatment groups displayed a skewed bimodal distribution with one smaller peak around the −50 degree orientation mark and one larger peak around the +50 degree orientation mark. This bimodal skew is attributed to the loss of rete ridges in the papillary dermis after a full-thickness wound. CO+DCN treated mice exhibited the highest collagen alignment around the +50 degree orientation mark compared to the other two treatments groups. The second measure investigated was the overall coherency of the collagen fibrils within each image. Coherency measure is between 0 and 1, where 0) indicates complete isotropy and 1 indicate complete alignment (Rezakhaniha et al., Biomech Model Mechanobiol. March 2012; 11 (3-4): 461-473). Similar to what was observed in the collagen alignment, CO+DCN treated mice exhibited the highest coherency rate compared to the two other groups while significantly higher than CO only treated mice (FIG. 13). These results show that CO+DCN mice are further developed and more mature in their collagen content and alignment.

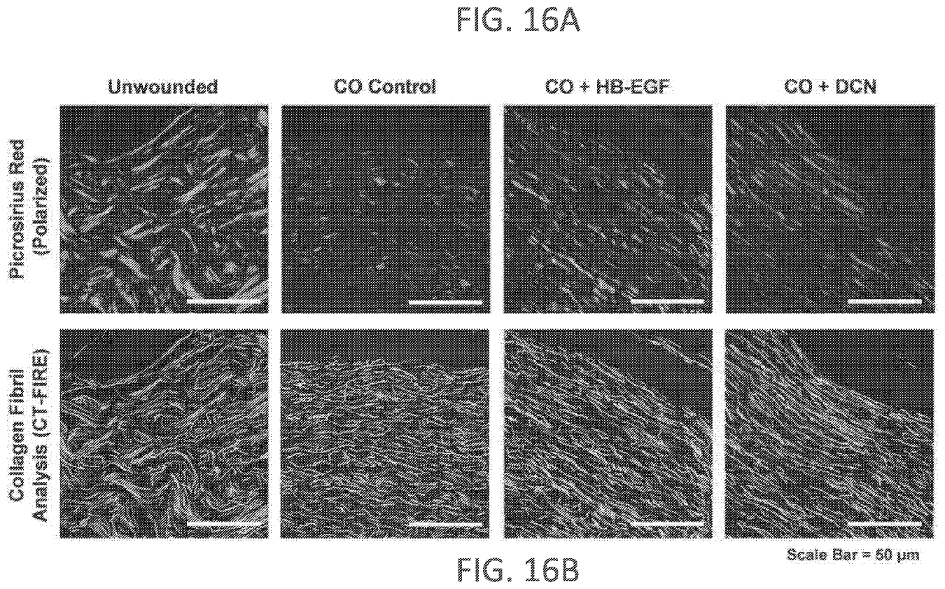

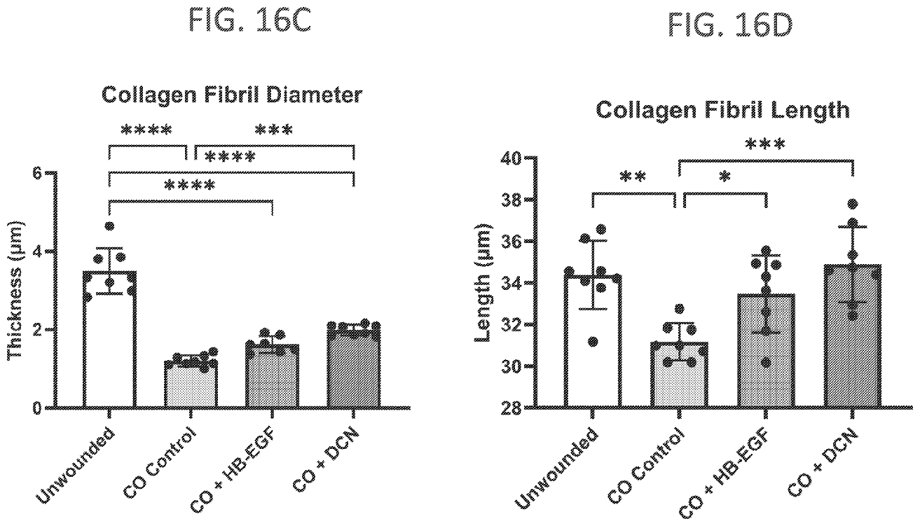

To further examine differences in collagen fibril modification, high resolution PSR images at 100× magnification were analyzed using CTFIRE for overall fibril diameter and length (FIGS. 16A-16B). CO+DCN-treated mice exhibited a significantly thicker diameter compared to only CO-treated mice (FIG. 16C). Additionally, both CO+DCN and CO+HB-EGF were significantly longer than only CO-treated mice and were very similar to the unwounded control (FIG. 16D). Altogether these results show that CO+DCN-treated mice are further developed and more mature in their collagen content and fibrillogenesis during the resolution phase of wound healing.

It will be apparent that the precise details of the methods or compositions described may be varied or modified without departing from the spirit of the described aspects of the disclosure. We claim all such modifications and variations that fall within the scope and spirit of the claims below.

1. A method of treating a wound in a subject, comprising administering to the subject a therapeutically effective amount of a first agent that promotes wound closure and a therapeutically effective amount of a second agent that inhibits scarring, wherein:
the first agent is administered to the subject prior to administration of the second agent; or
the first agent and the second agent are administered concurrently in a controlled delivery composition that permits rapid release of the first agent and delayed release of the second agent.

2. The method of claim 1, wherein:
the first agent comprises heparin binding EGF-like growth factor (HB-EGF), tenascin-C (TNC), a growth factor, a matricellular protein or a biologically active fragment of a matricellular protein, interleukin (IL)-1, IL-2, or IL-4; and/or
the second agent comprises decorin (DCN), a CXCR3 ligand, collagen type I, or IL-10.

3. The method of claim 2, wherein:
the growth factor is selected from epidermal growth factor (EGF), vascular endothelial growth factor (VEGF), a member of the fibroblast growth factor (FGF) family, transforming growth factor (TGF)-«, amphiregulin, and platelet-derived growth factor (PDGF); or
the matricellular protein is selected from secreted protein acidic and cysteine rich (SPARC), thrombospondin, laminin B1, and collagen type III.

4-5. (canceled)

6. The method of claim 2, wherein the CXCR3 ligand is selected from CXCL4, CXCL9, CXCL10 and CXCL11, or is selected from a biologically active fragment of CXCL4, CXCL9, CXCL10 and CXCL11.

7. The method of claim 1, wherein the first agent is administered at least one day prior to administration of the second agent.

8. The method of claim 7, wherein the first agent is administered at least 2 days, at least 3 days, at least 4 days, at least 5 days, at least 6 days, at least 1 week, at least 2 weeks or at least 3 weeks prior to administration of the second agent.

9. The method of claim 1, wherein the controlled delivery composition comprises a hydrogel and the hydrogel comprises:
the first agent; and
the second agent encapsulated in a coacervate,
wherein upon administration of the controlled delivery composition, the first agent is released from the hydrogel prior to release of the second agent from the coacervate.

10. The method of claim 1, wherein the controlled delivery composition comprises:
a first coacervate comprising the first agent;
a second coacervate comprising the second agent, wherein the second coacervate is encapsulated by lipids, thereby forming a lipo-coacervate,
wherein upon administration of the controlled delivery composition, the first agent is released from the first coacervate prior to release of the second agent from the lipo-coacervate.

11. The method of claim 10, wherein the lipids of the lipo-coacervate comprise:
cholesterol; and
at least one unsaturated lipid and/or at least one saturated lipid.

12. The method of claim 11, wherein:
the at least one unsaturated lipid is selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni); and/or
the at least one saturated lipid is select from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

13. The method of claim 11, wherein:
the lipids of the lipo-coacervate comprise cholesterol, DOPC and DSPG; or
the lipids of the lipo-coacervate comprise cholesterol, DPPC and DSPG.

14. (canceled)

15. The method of claim 1, wherein the controlled delivery composition comprises:
a first coacervate comprising the first agent, wherein the first coacervate is encapsulated by lipids, thereby forming a first lipo-coacervate; and
a second coacervate comprising the second agent, wherein the second coacervate is encapsulated by lipids, thereby forming a second lipo-coacervate,
wherein upon administration of the controlled delivery composition, the first agent is released from the first lipo-coacervate prior to release of the second agent from the second lipo-coacervate.

16. The method of claim 15, wherein:
the first lipo-coacervate comprises cholesterol and at least one unsaturated lipid selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni); and/or
the second lipo-coacervate comprises cholesterol and at least one saturated lipid selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

17. The method of claim 16, wherein the first lipo-coacervate further comprises a saturated lipid and/or cholesterol.

18. The method of claim 17, wherein:
the saturated lipid comprises 18:0 PG (DSPG);
the first lipo-coacervate comprises cholesterol, DOPC and DSPG; and/or
the second lipo-coacervate comprises cholesterol, DPPC and DSPG.

19-21. (canceled)

22. The method of claim 9, wherein the coacervate comprises poly(ethylene arginyl aspartate diglyceride) (PEAD) and heparin.

23. The method of claim 1, wherein the wound is a dermal wound or a mucosal wound.

24. The method of claim 23, wherein:
the dermal wound comprises a laceration, a puncture wound, an abrasion, a surgical wound, a burn, an ulcer or a pressure sore; or
the mucosal wound is in the nose, mouth, rectum, anus, vagina or lung.

25. (canceled)

26. The method of claim 1, wherein:
administration comprises topical administration; or
administration comprises injection at or near the site of the wound.

27. (canceled)

28. A controlled delivery composition for treating a wound, comprising:
a therapeutically effective amount of a first agent that promotes wound closure selected from heparin binding EGF-like growth factor (HB-EGF), tenascin-C (TNC), a growth factor, a matricellular protein, interleukin (IL)-1, IL-2 and IL-4; and
a therapeutically effective amount of a second agent that inhibits scarring selected from decorin (DCN), a CXCR3 ligand, collagen type I and IL-10,
wherein the controlled delivery composition permits rapid release of the first agent and delayed release of the second agent.

29. The controlled delivery composition of claim 28, wherein:
the growth factor is selected from epidermal growth factor (EGF), vascular endothelial growth factor (VEGF), a member of the fibroblast growth factor (FGF) family, transforming growth factor (TGF)-α, amphiregulin, and platelet-derived growth factor (PDGF);
the matricellular protein is selected from secreted protein acidic and cysteine rich (SPARC), thrombospondin, laminin B1, and collagen type III; and/or
the CXCR3 ligand is selected from CXCL4, CXCL9, CXCL10 and CXCL11.

30. The controlled delivery composition of claim 28, comprising a hydrogel, wherein the hydrogel comprises:
the first agent; and
the second agent encapsulated in a coacervate,
wherein upon administration of the controlled delivery composition, the first agent is released from the hydrogel prior to release of the second agent from the coacervate.

31. The controlled delivery composition of claim 28, wherein the controlled delivery composition comprises:
a first coacervate comprising the first agent;
a second coacervate comprising the second agent, wherein the second coacervate is encapsulated by lipids, thereby forming a lipo-coacervate,
wherein upon administration of the controlled delivery composition, the first agent is released from the first coacervate prior to release of the second agent from the lipo-coacervate.

32. The controlled delivery composition of claim 31, wherein the lipids of the lipo-coacervate comprise:
cholesterol; and
at least one unsaturated lipid and/or at least one saturated lipid.

33. The controlled delivery composition of claim 32, wherein:
the at least one unsaturated lipid is selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni); and/or
the at least one saturated lipid is select from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

34. The controlled delivery composition of claim 32, wherein:
the lipids of the lipo-coacervate comprise cholesterol, DOPC and DSPG; or
the lipids of the lipo-coacervate comprise cholesterol, DPPC and DSPG.

35. (canceled)

36. The controlled delivery composition of claim 28, wherein the controlled delivery composition comprises:
a first coacervate comprising the first agent, wherein the first coacervate is encapsulated by lipids, thereby forming a first lipo-coacervate; and
a second coacervate comprising the second agent, wherein the second coacervate is encapsulated by lipids, thereby forming a second lipo-coacervate,
wherein upon administration of the controlled delivery composition, the first agent is released from the first lipo-coacervate prior to release of the second agent from the second lipo-coacervate.

37. The controlled delivery composition of claim 36, wherein:
the first lipo-coacervate comprises cholesterol and at least one unsaturated lipid selected from 18:1 (Δ9-Cis) PC (DOPC), 18:1 (Δ9-Cis) PG (DOPG), 18:1 (Δ9-Cis) PE (DOPE), 16:0-18:1 PE (POPE), 16:0-18:1 PC (POPC), 16:0-18:1 PS (POPS) and 18:1 DGS-NTA (Ni); and/or
the second lipo-coacervate comprises cholesterol and at least one saturated lipid selected from 16:0 PC (DPPC), 18:0 PC (DSPC), 16:0 PS (DPPS), 18:0 PS (DSPS), 16:0 PE (DPPE), 18:0 PE (DSPE), 16:0 PG (DPPG), and 18:0 PG (DSPG).

38. The controlled delivery composition of claim 37, wherein the first lipo-coacervate further comprises a saturated lipid.

39. The controlled delivery composition of claim 38, wherein:
the saturated lipid comprises 18:0 PG (DSPG);
the first lipo-coacervate comprises cholesterol, DOPC and DSPG; and/or
the second lipo-coacervate comprises cholesterol, DPPC and DSPG.

40-42. (canceled)

43. The controlled delivery composition of claim 30, wherein the coacervate comprises poly(ethylene arginyl aspartate diglyceride) (PEAD) and heparin.

44. The controlled delivery composition of claim 31, wherein the first coacervate and/or the second coacervate comprises poly(ethylene arginyl aspartate diglyceride) (PEAD) and heparin.

45. The controlled delivery composition of claim 36, wherein the first coacervate and/or the second coacervate comprises poly(ethylene arginyl aspartate diglyceride) (PEAD) and heparin.

46. The method of claim 10, wherein the first coacervate and/or the second coacervate comprises poly(ethylene arginyl aspartate diglyceride) (PEAD) and heparin.

47. The method of claim 15, wherein the first coacervate and/or the second coacervate comprises poly(ethylene arginyl aspartate diglyceride) (PEAD) and heparin.
